SEQUENCE LISTING (299 sequences)

SEQ ID NO: 1 - protein, 5 aa, Synthetic construct
NYAMS

SEQ ID NO: 2 - protein, 17 aa, Synthetic construct
AINNNGDDTYYLDTVKD

SEQ ID NO: 3 - protein, 5 aa, Synthetic construct
QGGAY

SEQ ID NO: 4 - protein, 7 aa, Synthetic construct
TYGMGVG

SEQ ID NO: 5 - protein, 16 aa, Synthetic construct
NIWWYDAKYYNSDLKS

SEQ ID NO: 6 - protein, 8 aa, Synthetic construct
MGLAWFAY

SEQ ID NO: 7 - protein, 7 aa, Synthetic construct
IYGMGVG

SEQ ID NO: 8 - protein, 16 aa, Synthetic construct
NIWWNDDKYYNSALKS

SEQ ID NO: 9 - protein, 8 aa, Synthetic construct
IGYFYFDY

SEQ ID NO: 10 - protein, 5 aa, Synthetic construct
SYAMS

SEQ ID NO: 11 - protein, 17 aa, Synthetic construct
AINSNGGTTYYPDTMKD

SEQ ID NO: 12 - protein, 13 aa, Synthetic construct
HNGGYENYGWFAY

SEQ ID NO: 13 - protein, 5 aa, Synthetic construct
GYWMH

SEQ ID NO: 14 - protein, 17 aa, Synthetic construct
AIYPGNSDTNYNQKFKG

SEQ ID NO: 15 - protein, 10 aa, Synthetic construct
SGDLTGGLAY

SEQ ID NO: 16 - protein, 5 aa, Synthetic construct
SYWMH

SEQ ID NO: 17 - protein, 17 aa, Synthetic construct
EIDPSDSYTYYNQKFRG

SEQ ID NO: 18 - protein, 15 aa, Synthetic construct
SNLGDGHYRFPAFPY

SEQ ID NO: 19 - protein, 17 aa, Synthetic construct
TIDPSDSETHYNLQFKD

SEQ ID NO: 20 - protein, 15 aa, Synthetic construct
GAFYSSYSYWAWFAY

SEQ ID NO: 21 - protein, 5 aa, Synthetic construct
DYSMH

SEQ ID NO: 22 - protein, 17 aa, Synthetic construct
WINTETGEPTYADDFKG

SEQ ID NO: 23 - skipped

SEQ ID NO: 24 - protein, 16 aa, Synthetic construct
NIWWHDDKYYNSALKS

SEQ ID NO: 25 - protein, 14 aa, Synthetic construct
IAPRYNKYEGFFAF

SEQ ID NO: 26 - protein, 5 aa, Synthetic construct
DYEMH

SEQ ID NO: 27 - protein, 17 aa, Synthetic construct
ALDPKTGDTAYSQKFKG

SEQ ID NO: 28 - protein, 6 aa, Synthetic construct
FYSYTY

SEQ ID NO: 29 - protein, 5 aa, Synthetic construct
INAMN

SEQ ID NO: 30 - protein, 19 aa, Synthetic construct
RIRSESNNYATYYGDSVKD

SEQ ID NO: 31 - protein, 8 aa, Synthetic construct
EVTTSFAY

SEQ ID NO: 32 - protein, 5 aa, Synthetic construct
ASAMN

SEQ ID NO: 33 - protein, 19 aa, Synthetic construct
RIRSKSNNYAIYYADSVKD

SEQ ID NO: 34 - protein, 12 aa, Synthetic construct
DPGYYGNPWFAY

SEQ ID NO: 35 - protein, 463 aa, Synthetic construct
MNFGLTLIFLVLTLKGVQCEVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 114 aa, Synthetic construct
EVHLVESGGGLVKPGGSLKLSCAASGFTFSNYAMSWVRQTPEKRLEWVAAINNNGDDTYYLDTVKDRFTISRDNAKNTLYLQMSSLRSEDTALYYCVRQGGAYWGQGTLVTVSA

SEQ ID NO: 37 - protein, 470 aa, Synthetic construct
MGWNWIFILILSVTTGVHSEVQLQQSGPELVKPGASVKISCKASGYSFTGYYMHWVKQSPEKSLEWIGEINPSTGGTTYNQKFKAKATLTVDKSSSTAYMQLKSLTSEDSAVYYCARRGGLTGTSFFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 118 aa, Synthetic construct
QVTLKESGPGILQPSQTLSLTCSFSGFSLSTYGMGVGWIRQPSGMGLEWLANIWWYDAKYYNSDLKSRLTISKDTSNNQVFLKISSVDTSDTATYYCAQMGLAWFAYWGQGTLVTVSA

SEQ ID NO: 39 - protein, 118 aa, Synthetic construct
QVTLKESGPGILQPSQTLSLTCSFSGFSLSIYGMGVGWIRQPSGKGLEWLANIWWNDDKYYNSALKSRLTISKDTSNNQVFLKISSVDTADTATYYCAQIGYFYFDYWGQGTTLTVSS

SEQ ID NO: 40 - protein, 471 aa, Synthetic construct
MNFGLTLIFLVLTLKGVQCEVQLVESGGDLVKPGGTLKLSCAASGSTFSNYAMSWVRQTPEKRLEWVAAIDSNGGTTYYPDTMKDRFTISRDNAKNTLYLQMNSLRSEDTAFYHCTRHNGGYENYGWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 122 aa, Synthetic construct
EVQLVESGGDLVKPGGSLKLSCAASGFTFSSYAMSWVRQTPEKRLEWVAAINSNGGTTYYPDTMKDRFTISRDNAKNTLYLQMSSLRSEDSALYYCTRHNGGYENYGWFAYWGQGTLVTVSA

SEQ ID NO: 42 - protein, 470 aa, Synthetic construct
MESNWILPFILSVASGVYSEVQLQQSGTVLARPGASVKMSCKASGYTFTGYWMRWVKQRPGQGLEWIGAIYPGNSDTTYNQKFKGKAKLTAVTSVSTAYMELSSLTNEDSAVYYCSRSGDLTGGFAYWGQGTLVTVSTAKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 119 aa, Synthetic construct
EVQLQQSGTVLARPGASVKMSCKASGYTFTGYWMHWVKQRPGQGLEWIGAIYPGNSDTNYNQKFKGKAKLTAVTSASTAYMELSSLTNEDAAVYHCTRSGDLTGGLAYWGQGTLVTVSA

SEQ ID NO: 44 - protein, 124 aa, Synthetic construct
QVQLQQPGAELVKPGASVKLSCKASGYTFTSYWMHWVKQRPGQGLEWIGEIDPSDSYTYYNQKFRGKATLTVDKSSNTAYMQLSSLTSEDSAVYYCSRSNLGDGHYRFPAFPYWGQGTLVTVSA

SEQ ID NO: 45 - protein, 124 aa, Synthetic construct
QVQLQQPGAELVKPGASVKLSCKASGYTFTSYWMHWVKQRPGQGLEWIGTIDPSDSETHYNLQFKDTATLTVDKSSSTAYMQLSSLTSEDSAVYYCIRGAFYSSYSYWAWFAYWGQGTLVTVSA

SEQ ID NO: 46 - protein, 111 aa, Synthetic construct
QIQLEQSGPELKKPGETVKISCKASGYIFRDYSMHWVKQAPGKGLKWMGWINTETGEPTYADDFKGRFAFSLETSASTAYLQINNLKNEDTATYFCTSLYWGQGTLVTVSA

SEQ ID NO: 47 - protein, 124 aa, Synthetic construct
QVTLKESGPGILQPSQTLSLTCSFSGFSLSTYGMGVGWIRQPSGKGLEWLANIWWHDDKYYNSALKSRLTISKDISNNQVFLKISSVDTADTATYYCAQIAPRYNKYEGFFAFWGQGTLVTVSA

SEQ ID NO: 48 - protein, 115 aa, Synthetic construct
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 49 - protein, 119 aa, Synthetic construct
EVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSA

SEQ ID NO: 50 - protein, 123 aa, Synthetic construct
EVQLVETGGGLVQPKGSLKLSCAASGFTFNASAMNWVRQAPGKGLEWVARIRSKSNNYAIYYADSVKDRFTISRDDSQSMLYLQMNNLKTEDTAMYYCVRDPGYYGNPWFAYWGQGTLVTVSA

SEQ ID NO: 51 - protein, 115 aa, Synthetic construct
QVQLVESGAEVKKPGASVKVSCKASGYTFTDYEMHWVRQAPGQGLEWMGALDPKTGDTAYSQKFKGRVTITADESTSTAYMELSSLRSEDTAVYYCARFYSYTYWGQGTLVTVSS

SEQ ID NO: 52 - protein, 115 aa, Synthetic construct
QVQLVESGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 53 - protein, 115 aa, Synthetic construct
QVQLVESGAEVKKPGASVKVSCKASGYTFTDYEMHWVRQAPGQGLEWMGALDPKTGDTAYSQKFKGRVTLTADKSTSTAYMELSSLRSEDTAVYYCTRFYSYTYWGQGTLVTVSS

SEQ ID NO: 54 - protein, 115 aa, Synthetic construct
QVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 55 - protein, 115 aa, Synthetic construct
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 56 - protein, 115 aa, Synthetic construct
QVQLVQSGAEVKKPGASVKVSCKASGYTFTDYEMHWVRQAPGQGLEWMGALDPKTGDTAYSQKFKGRVTLTADKSTSTAYMELSSLRSEDTAVYYCTRFYSYTYWGQGTLVTVSS

SEQ ID NO: 57 - protein, 115 aa, Synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 58 - protein, 16 aa, Synthetic construct
KSSQSLLDSDGKTYLN

SEQ ID NO: 59 - protein, 7 aa, Synthetic construct
LVSKLDS

SEQ ID NO: 60 - protein, 9 aa, Synthetic construct
WQGTHFPLT

SEQ ID NO: 61 - protein, 11 aa, Synthetic construct
KASQDINNYLS

SEQ ID NO: 62 - protein, 7 aa, Synthetic construct
RANRLVD

SEQ ID NO: 63 - protein, 10 aa, Synthetic construct
LQCDEFPPWT

SEQ ID NO: 64 - protein, 16 aa, Synthetic construct
RSSQSLVHSNGNTYLH

SEQ ID NO: 65 - protein, 7 aa, Synthetic construct
KVSNRFS

SEQ ID NO: 66 - protein, 9 aa, Synthetic construct
SQSTHVPWT

SEQ ID NO: 67 - protein, 11 aa, Synthetic construct
KASQDINKNII

SEQ ID NO: 68 - protein, 7 aa, Synthetic construct
YTSTLQP

SEQ ID NO: 69 - protein, 9 aa, Synthetic construct
LQYDNLPRT

SEQ ID NO: 70 - protein, 16 aa, Synthetic construct
RSSKSLLHSNGITYLY

SEQ ID NO: 71 - protein, 7 aa, Synthetic construct
QMSNLAS

SEQ ID NO: 72 - protein, 9 aa, Synthetic construct
AQNLELPYT

SEQ ID NO: 73 - protein, 11 aa, Synthetic construct
RASHSISNFLH

SEQ ID NO: 74 - protein, 7 aa, Synthetic construct
YASQSIS

SEQ ID NO: 75 - protein, 9 aa, Synthetic construct
QQSNIWSLT

SEQ ID NO: 76 - protein, 15 aa, Synthetic construct
RASESVEYYGTSLMQ

SEQ ID NO: 77 - protein, 7 aa, Synthetic construct
GASNVES

SEQ ID NO: 78 - protein, 9 aa, Synthetic construct
QQSRKVPYT

SEQ ID NO: 79 - protein, 16 aa, Synthetic construct
KSSQSLLHSDGKTFLN

SEQ ID NO: 80 - protein, 7 aa, Synthetic construct
LVSRLDS

SEQ ID NO: 81 - protein, 9 aa, Synthetic construct
CQGTHFPRT

SEQ ID NO: 82 - protein, 16 aa, Synthetic construct
RSSQSIVHSNGNTYLE

SEQ ID NO: 83 - protein, 9 aa, Synthetic construct
FQGSHVPWT

SEQ ID NO: 84 - protein, 9 aa, Synthetic construct
SQNTHVPPT

SEQ ID NO: 85 - protein, 16 aa, Synthetic construct
RSSQSLVHSNANTYLH

SEQ ID NO: 86 - protein, 16 aa, Synthetic construct
RSSQSLVHSNDNTYLH

SEQ ID NO: 87 - protein, 16 aa, Synthetic construct
RSSQSLVHSNENTYLH

SEQ ID NO: 88 - protein, 16 aa, Synthetic construct
RSSQSLVHSNFNTYLH

SEQ ID NO: 89 - protein, 16 aa, Synthetic construct
RSSQSLVHSNHNTYLH

SEQ ID NO: 90 - protein, 16 aa, Synthetic construct
RSSQSLVHSNNNTYLH

SEQ ID NO: 91 - protein, 16 aa, Synthetic construct
RSSQSLVHSNTNTYLH

SEQ ID NO: 92 - protein, 16 aa, Synthetic construct
RSSQSLVHSNQNTYLH

SEQ ID NO: 93 - protein, 16 aa, Synthetic construct
RSSQSLVHSNINTYLH

SEQ ID NO: 94 - protein, 16 aa, Synthetic construct
RSSQSLVHSNKNTYLH

SEQ ID NO: 95 - protein, 16 aa, Synthetic construct
RSSQSLVHSNLNTYLH

SEQ ID NO: 96 - protein, 16 aa, Synthetic construct
RSSQSLVHSNSNTYLH

SEQ ID NO: 97 - protein, 16 aa, Synthetic construct
RSSQSLVHSNWNTYLH

SEQ ID NO: 98 - protein, 16 aa, Synthetic construct
RSSQSLVHSNYNTYLH

SEQ ID NO: 99 - protein, 16 aa, Synthetic construct
RSSQSLVHSNRNTYLH

SEQ ID NO: 100 - protein, 16 aa, Synthetic construct
RSSQSLVHSNVNTYLH

SEQ ID NO: 101 - protein, 16 aa, Synthetic construct
RSSQSLVHSNPNTYLH

SEQ ID NO: 102 - protein, 16 aa, Synthetic construct
KSSKSLLHSNGNTYLN

SEQ ID NO: 103 - protein, 7 aa, Synthetic construct
WMSNLAS

SEQ ID NO: 104 - protein, 9 aa, Synthetic construct
MQHIEYPFT

SEQ ID NO: 105 - protein, 16 aa, Synthetic construct
RSSKSLLHSYDITYLY

SEQ ID NO: 106 - protein, 9 aa, Synthetic construct
AQNLELPPT

SEQ ID NO: 107 - protein, 10 aa, Synthetic construct
SASSSVSYMY

SEQ ID NO: 108 - protein, 7 aa, Synthetic construct
DTSNLAS

SEQ ID NO: 109 - protein, 9 aa, Synthetic construct
QQWSSYPLT

SEQ ID NO: 110 - protein, 238 aa, Synthetic construct
MSPAQFLFLLVLWIRETNGDVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 112 aa, Synthetic construct
DVVMTQSPLTLSITIGQPASISCKSSQSLLDSDGKTYLNWLLQRPGQSPKRLIYLVSKLDSGVPDRFTGSGSGTDFSLKISRVEAEDLGIYYCWQGTHFPLTFGAGTKLELK

SEQ ID NO: 112 - protein, 238 aa, Synthetic construct
MSPVQFLFLLMLWIQETNGDVVMTQTPLSLSVTIGQPASISCKSSQSLLYSNGKTYLNWLQQRPGQAPKHLMYQVSKLDPGIPDRFSGSGSETDFTLKISRVEAEDLGVYYCLQSTYYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 108 aa, Synthetic construct
DIKMTQSPSSMYASLGERVTITCKASQDINNYLSWFQQKPGKSPKTLIYRANRLVDGVPSRFSGSGSGQDYSLTISSLEYEDMGINYCLQCDEFPPWTFGGGTKLEIK

SEQ ID NO: 114 - protein, 112 aa, Synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 115 - protein, 234 aa, Synthetic construct
MRPSIQFLGLLLFWLHGVQCDIQMTQSPSSLSASLGGKVTITCKASQDINKNIVWYQHKPGKGPRLLIWYTSTLQPGIPSRFSGSGSGRDYSFSISNLEPEDIATYYCLQYDNLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 107 aa, Synthetic construct
DIQMTQSPSSLSASLGGKVTITCKASQDINKNIIWYQHKPGKGPRLLIWYTSTLQPGIPSRFSGSGSGRDYSFSISNLEPEDIATYYCLQYDNLPRTFGGGTKLEIK

SEQ ID NO: 117 - protein, 239 aa, Synthetic construct
MRFSAQLLGLLVLWIPGSTADIVMTQAAFSNPVTLGTSTSISCRSSKSLLHSNGITYLYWYLQKPGQSPQLLIYQMSNLASGVPDRFSSSGSGTDFTLRISRVEAEDVGVYYCAQNLELPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 112 aa, Synthetic construct
DIVMTQAAFSNPVTLGTSASISCRSSKSLLHSNGITYLYWFLQKPGQSPQLLIYQMSNLASGVPDRFSSSGSGTDFTLRISRVEAEDVGVYYCAQNLELPYTFGSGTKLEIK

SEQ ID NO: 119 - protein, 107 aa, Synthetic construct
DIVLTQSPATLSVTPGDRVSLSCRASHSISNFLHWYPQKSHESPRLLIKYASQSISGIPSRFSGNGSGTDFTLSINSVETEDFGMYFCQQSNIWSLTFGAGTKLELK

SEQ ID NO: 120 - protein, 111 aa, Synthetic construct
DIVLTQSPTSLAVSLGQSVTISCRASESVEYYGTSLMQWYQQKPGQPPKLLIYGASNVESGVPARFSGSGSGTDFSLNIHPVEEDDIAMYFCQQSRKVPYTFGSGTKLEIK

SEQ ID NO: 121 - protein, 112 aa, Synthetic construct
DVVMTQTPLTLSVTLGQPASISCKSSQSLLHSDGKTFLNWLLQRPGQSPKRLIYLVSRLDSGVPDRFTGSGSGTDFTLKISRVEAEDLGVYYCCQGTHFPRTFGGGTRLEIK

SEQ ID NO: 122 - protein, 112 aa, Synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 123 - protein, 112 aa, Synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 124 - protein, 112 aa, Synthetic construct
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 125 - protein, 112 aa, Synthetic construct
DVVMTQSPLSLPVTPGEPASISCRSSQSLVHSNDNTYLHWYLQKPGQSPQLLIYKVSNRFSGVPDRFSGSGSGTDFTLKISRVEAEDVGVYYCSQNTHVPPTFGQGTKLEIK

SEQ ID NO: 126 - protein, 112 aa, Synthetic construct
DVVMTQSPLSLPVTPGEPASISCRSSQSLVHSNENTYLHWYLQKPGQSPQLLIYKVSNRFSGVPDRFSGSGSGTDFTLKISRVEAEDVGVYYCSQNTHVPPTFGQGTKLEIK

SEQ ID NO: 127 - protein, 112 aa, Synthetic construct
DVVMTQSPLSLPVTPGEPASISCRSSQSLVHSNFNTYLHWYLQKPGQSPQLLIYKVSNRFSGVPDRFSGSGSGTDFTLKISRVEAEDVGVYYCSQNTHVPPTFGQGTKLEIK

SEQ ID NO: 128 - protein, 112 aa, Synthetic construct
DVVMTQSPLSLPVTPGEPASISCRSSQSLVHSNHNTYLHWYLQKPGQSPQLLIYKVSNRFSGVPDRFSGSGSGTDFTLKISRVEAEDVGVYYCSQNTHVPPTFGQGTKLEIK

SEQ ID NO: 129 - protein, 112 aa, Synthetic construct
DVVMTQSPLSLPVTPGEPASISCRSSQSLVHSNNNTYLHWYLQKPGQSPQLLIYKVSNRFSGVPDRFSGSGSGTDFTLKISRVEAEDVGVYYCSQNTHVPPTFGQGTKLEIK

SEQ ID NO: 130 - protein, 112 aa, Synthetic construct
DVVMTQSPLSLPVTPGEPASISCRSSQSLVHSNTNTYLHWYLQKPGQSPQLLIYKVSNRFSGVPDRFSGSGSGTDFTLKISRVEAEDVGVYYCSQNTHVPPTFGQGTKLEIK

SEQ ID NO: 131 - protein, 112 aa, Synthetic construct
DVVMTQSPLSLPVTPGEPASISCRSSQSLVHSNQNTYLHWYLQKPGQSPQLLIYKVSNRFSGVPDRFSGSGSGTDFTLKISRVEAEDVGVYYCSQNTHVPPTFGQGTKLEIK

SEQ ID NO: 132 - protein, 112 aa, Synthetic construct
DVVMTQSPLSLPVTPGEPASISCRSSQSLVHSNINTYLHWYLQKPGQSPQLLIYKVSNRFSGVPDRFSGSGSGTDFTLKISRVEAEDVGVYYCSQNTHVPPTFGQGTKLEIK

SEQ ID NO: 133 - protein, 112 aa, Synthetic construct
DVVMTQSPLSLPVTPGEPASISCRSSQSLVHSNKNTYLHWYLQKPGQSPQLLIYKVSNRFSGVPDRFSGSGSGTDFTLKISRVEAEDVGVYYCSQNTHVPPTFGQGTKLEIK

SEQ ID NO: 134 - protein, 112 aa, Synthetic construct
DVVMTQSPLSLPVTPGEPASISCRSSQSLVHSNLNTYLHWYLQKPGQSPQLLIYKVSNRFSGVPDRFSGSGSGTDFTLKISRVEAEDVGVYYCSQNTHVPPTFGQGTKLEIK

SEQ ID NO: 135 - protein, 112 aa, Synthetic construct
DVVMTQSPLSLPVTPGEPASISCRSSQSLVHSNSNTYLHWYLQKPGQSPQLLIYKVSNRFSGVPDRFSGSGSGTDFTLKISRVEAEDVGVYYCSQNTHVPPTFGQGTKLEIK

SEQ ID NO: 136 - protein, 112 aa, Synthetic construct
DVVMTQSPLSLPVTPGEPASISCRSSQSLVHSNWNTYLHWYLQKPGQSPQLLIYKVSNRFSGVPDRFSGSGSGTDFTLKISRVEAEDVGVYYCSQNTHVPPTFGQGTKLEIK

SEQ ID NO: 137 - protein, 112 aa, Synthetic construct
DVVMTQSPLSLPVTPGEPASISCRSSQSLVHSNYNTYLHWYLQKPGQSPQLLIYKVSNRFSGVPDRFSGSGSGTDFTLKISRVEAEDVGVYYCSQNTHVPPTFGQGTKLEIK

SEQ ID NO: 138 - protein, 112 aa, Synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 139 - protein, 112 aa, Synthetic construct
DVVMTQSPLSLPVTPGEPASISCRSSQSLVHSNVNTYLHWYLQKPGQSPQLLIYKVSNRFSGVPDRFSGSGSGTDFTLKISRVEAEDVGVYYCSQNTHVPPTFGQGTKLEIK

SEQ ID NO: 140 - protein, 112 aa, Synthetic construct
DVVMTQSPLSLPVTPGEPASISCRSSQSLVHSNPNTYLHWYLQKPGQSPQLLIYKVSNRFSGVPDRFSGSGSGTDFTLKISRVEAEDVGVYYCSQNTHVPPTFGQGTKLEIK

SEQ ID NO: 141 - protein, 112 aa, Synthetic construct
DIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIK

SEQ ID NO: 142 - protein, 112 aa, Synthetic construct
DIVMTQAAFSNPVTLGTSASISCRSSKSLLHSYDITYLYWYLQKPGQSPQLLIYQMSNLASGVPDRFSSSGSGTDFTLRISRVEAEDVGVYYCAQNLELPPTFGGGTKLEIK

SEQ ID NO: 143 - protein, 106 aa, Synthetic construct
QIVLTQSPAIMSAFPGEKVTMTCSASSSVSYMYWYQQKSGSSPRLLIYDTSNLASGVPVRFSGSGSGTSYSLTISRMEAEDAATYYCQQWSSYPLTFGGGTELELK

SEQ ID NO: 144 - protein, 112 aa, Synthetic construct
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 145 - protein, 5 aa, Synthetic construct
SYWIA

SEQ ID NO: 146 - protein, 17 aa, Synthetic construct
IIFPGDSDTRYSPSFQG

SEQ ID NO: 147 - protein, 8 aa, Synthetic construct
TREGYFDY

SEQ ID NO: 148 - protein, 5 aa, Synthetic construct
NYWIA

SEQ ID NO: 149 - protein, 17 aa, Synthetic construct
IIYPGDSDTRYSPSFQG

SEQ ID NO: 150 - protein, 5 aa, Synthetic construct
NYWIG

SEQ ID NO: 151 - protein, 8 aa, Synthetic construct
TREGFFDY

SEQ ID NO: 152 - protein, 117 aa, Synthetic construct
EVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSS

SEQ ID NO: 153 - protein, 117 aa, Synthetic construct
EVQLVQSGAEVKKPGESLKISCKGSGYSFTNYWIAWVRQMPGKGLEWMGIIYPGDSDTRYSPSFQGQVTISADKSIRTAYLQWSSLKASDTAMYYCARTREGYFDYWGQGTLVTVSS

SEQ ID NO: 154 - protein, 117 aa, Synthetic construct
EVQLVQSGADVTKPGESLKISCKVSGYRFTNYWIGWMRQMSGKGLEWMGIIYPGDSDTRYSPSFQGHVTISADKSINTAYLRWSSLKASDTAIYYCARTREGFFDYWGQGTPVTVSS

SEQ ID NO: 155 - protein, 12 aa, Synthetic construct
RAVQSVSSSYLA

SEQ ID NO: 156 - protein, 7 aa, Synthetic construct
GASSRAT

SEQ ID NO: 157 - protein, 8 aa, Synthetic construct
QQYGSSPT

SEQ ID NO: 158 - protein, 12 aa, Synthetic construct
RASQSVSSSYLA

SEQ ID NO: 159 - protein, 107 aa, Synthetic construct
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIK

SEQ ID NO: 160 - protein, 107 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGGGTKVEIK

SEQ ID NO: 161 - protein, 107 aa, Synthetic construct
EILLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKVEIK

SEQ ID NO: 162 - skipped

SEQ ID NO: 163 - protein, 6 aa, Synthetic construct
GGSGGS

SEQ ID NO: 164 - protein, 9 aa, Synthetic construct
GGSGGSGGS

SEQ ID NO: 165 - protein, 12 aa, Synthetic construct
GGSGGSGGSGGS

SEQ ID NO: 166 - protein, 15 aa, Synthetic construct
GGSGGSGGSGGSGGS

SEQ ID NO: 167 - protein, 4 aa, Synthetic construct
GGGS

SEQ ID NO: 168 - protein, 8 aa, Synthetic construct
GGGSGGGS

SEQ ID NO: 169 - protein, 12 aa, Synthetic construct
GGGSGGGSGGGS

SEQ ID NO: 170 - protein, 16 aa, Synthetic construct
GGGSGGGSGGGSGGGS

SEQ ID NO: 171 - protein, 20 aa, Synthetic construct
GGGSGGGSGGGSGGGSGGGS

SEQ ID NO: 172 - protein, 5 aa, Synthetic construct
GGGGS

SEQ ID NO: 173 - protein, 10 aa, Synthetic construct
GGGGSGGGGS

SEQ ID NO: 174 - protein, 15 aa, Synthetic construct
GGGGSGGGGSGGGGS

SEQ ID NO: 175 - protein, 20 aa, Synthetic construct
GGGGSGGGGSGGGGSGGGGS

SEQ ID NO: 176 - protein, 25 aa, Synthetic construct
GGGGSGGGGSGGGGSGGGGSGGGGS

SEQ ID NO: 177 - protein, 18 aa, Synthetic construct
GSTSGSGKPGSGEGSTKG

SEQ ID NO: 178 - protein, 20 aa, Synthetic construct
EAAAKEAAAKEAAAKEAAAK

SEQ ID NO: 179 - protein, 19 aa, Synthetic construct
GGGGSGGGGSGGGGSVDGF

SEQ ID NO: 180 - protein, 9 aa, Synthetic construct
ASGGGGSAS

SEQ ID NO: 181 - protein, 42 aa, Synthetic construct
AAAIEVMYPPPYLDNEKSNGTIIHVKGKHLCPSPLFPGPSKP

SEQ ID NO: 182 - protein, 12 aa, Synthetic construct
ESKYGPPCPSCP

SEQ ID NO: 183 - protein, 12 aa, Synthetic construct
ESKYGPPCPPCP

SEQ ID NO: 184 - protein, 12 aa, Synthetic construct
ESKYGPPCPPCP

SEQ ID NO: 185 - protein, 12 aa, Synthetic construct
EPKSCDKTHTCP

SEQ ID NO: 186 - protein, 86 aa, Synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 187 - protein, 45 aa, Synthetic construct
TTTPAPRPPTPAPTIALQPLSLRPEACRPAAGGAVHTRGLDFACD

SEQ ID NO: 188 - protein, 132 aa, Synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 189 - protein, 34 aa, Synthetic construct
ACPTGLYTHSGECCKACNLGEGVAQPCGANQTVC

SEQ ID NO: 190 - protein, 20 aa, Synthetic construct
AVGQDTQEVIVVPHSLPFKV

SEQ ID NO: 191 - protein, 176 aa, Synthetic construct
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 192 - protein, 65 aa, Synthetic construct
RTLVTFKDVFVDFTREEWKLLDTAQQIVYRNVMLENYKNLVSLGYQLTKPDVILRLEKGEEPWLV

SEQ ID NO: 193 - protein, 45 aa, Synthetic construct
RTLVTFKDVFVDFTREEWKLLDTAQQIVYRNVMLENYKNLVSLGY

SEQ ID NO: 194 - protein, 531 aa, Synthetic construct
EASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 195 - DNA, 195 bp, Synthetic construct
  source 1..195: mol_type = other DNA
agaaccctggtcaccttcaaggacgtgttcgtggacttcacccgggaagagtggaagctgctggatacagcccagcagatcgtgtaccggaacgtgatgctggaaaactacaagaatctggtgtccctgggctaccagctgaccaagcctgacgtgatcctgcggctggaaaagggcgaagaaccttggctggtg

SEQ ID NO: 196 - DNA, 135 bp, Synthetic construct
  source 1..135: mol_type = other DNA
agaaccctggtcaccttcaaggacgtgttcgtggacttcacccgggaagagtggaagctgctggatacagcccagcagatcgtgtaccggaacgtgatgctggaaaactacaagaatctggtgtccctgggctac

SEQ ID NO: 197 - protein, 10 aa, Synthetic construct
DEMEECSQHL

SEQ ID NO: 198 - protein, 9 aa, Synthetic construct
EDVVPCSMG

SEQ ID NO: 199 - protein, 10 aa, Synthetic construct
DEMEECSQHL

SEQ ID NO: 200 - protein, 9 aa, Synthetic construct
EDVVPCSMG

SEQ ID NO: 201 - protein, 10 aa, Synthetic construct
DEMEECSQHL

SEQ ID NO: 202 - protein, 9 aa, Synthetic construct
EDVVPCSMG

SEQ ID NO: 203 - protein, 10 aa, Synthetic construct
DEMEECSQHL

SEQ ID NO: 204 - protein, 9 aa, Synthetic construct
EDVVPCSMG

SEQ ID NO: 205 - protein, 10 aa, Synthetic construct
EDVVCCHSIY

SEQ ID NO: 206 - protein, 14 aa, Synthetic construct
LYQEFDEMEECSQH

SEQ ID NO: 207 - protein, 60 aa, Synthetic construct
FNVLMVHKRSHTGERPLQCEICGFTCRQKGNLLRHIKLHTGEKPFKCHLCNYACQRRDAL

SEQ ID NO: 208 - DNA, 31 bp, Synthetic construct
  source 1..31: mol_type = other DNA
agagggtatataatggaagctcgacttccag

SEQ ID NO: 209 - DNA, 52 bp, Synthetic construct
  source 1..52: mol_type = other DNA
gggaatttccggggactttccgggaatttccggggactttccgggaatttcc

SEQ ID NO: 210 - DNA, 52 bp, Synthetic construct; identical to SEQ ID NO: 209
  source 1..52: mol_type = other DNA

SEQ ID NO: 211 - DNA, 90 bp, Synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 212 - DNA, 115 bp, Synthetic construct
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 213 - DNA, 115 bp, Synthetic construct
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 214 - DNA, 48 bp, Synthetic construct
  source 1..48: mol_type = other DNA
tgagtcagtgactcagtgagtcagtgactcagtgagtcagtgactcag

SEQ ID NO: 215 - DNA, 120 bp, Synthetic construct
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 216 - DNA, 32 bp, Synthetic construct
  source 1..32: mol_type = other DNA
gtctagacgtctagacgtctagacgtctagac

SEQ ID NO: 217 - DNA, 24 bp, Synthetic construct
  source 1..24: mol_type = other DNA
cagacacagacacagacacagaca

SEQ ID NO: 218 - DNA, 65 bp, Synthetic construct
  source 1..65: mol_type = other DNA
ggatccggtactcgagatctgcgatctaagtaagcttggcattccggtactgttggtaaagccac

SEQ ID NO: 219 - DNA, 66 bp, Synthetic construct
  source 1..66: mol_type = other DNA
gggactttccactggggactttccactggggactttccactggggactttccactggggactttcc

SEQ ID NO: 220 - DNA, 51 bp, Synthetic construct
  source 1..51: mol_type = other DNA
agacgctagcggggggctataaaagggggtgggggcgttcgtcctcactct

SEQ ID NO: 221 - DNA, 140 bp, Synthetic construct
  source 1..140: mol_type = other DNA
gggactttccactggggactttccactggggactttccactggggactttccactggggactttccactcctgcaggagctggcgcgccagacgctagcggggggctataaaagggggtgggggcgttcgtcctcactct

SEQ ID NO: 222 - DNA, 25 bp, Synthetic construct
  source 1..25: mol_type = other DNA
tctagagggtatataatgggggcca

SEQ ID NO: 223 - DNA, 588 bp, Synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 224 - DNA, 1179 bp, Synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 225 - DNA, 544 bp, Synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 226 - DNA, 399 bp, Synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 227 - DNA, 511 bp, Synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 228 - DNA, 408 bp, Synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 229 - DNA, 344 bp, Synthetic construct
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 230 - DNA, 1229 bp, Synthetic construct
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 231 - DNA, 1179 bp, Synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 232 - DNA, 1718 bp, Synthetic construct
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 233 - DNA, 212 bp, Synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 234 - DNA, 2379 bp, Synthetic construct
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 235 - DNA, 529 bp, Synthetic construct
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 236 - DNA, 794 bp, Synthetic construct
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 237 - DNA, 532 bp, Synthetic construct
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 238 - DNA, 701 bp, Synthetic construct
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 239 - DNA, 465 bp, Synthetic construct
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 240 - DNA, 538 bp, Synthetic construct
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 241 - DNA, 1091 bp, Synthetic construct
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 242 - DNA, 18 bp, Synthetic construct
  source 1..18: mol_type = other DNA
ggcgtagccgatgtcgcg

SEQ ID NO: 243 - DNA, 172 bp, Synthetic construct
  source 1..172: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcg

SEQ ID NO: 244 - DNA, 243 bp, Synthetic construct
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 245 - protein, 22 aa, Synthetic construct
MCHQQLVISWFSLVFLASPLVA

SEQ ID NO: 246 - DNA, 66 bp, Synthetic construct
  source 1..66: mol_type = other DNA
atgtgtcaccagcagctcgttatatcctggtttagtttggtgtttctcgcttcacccctggtggca

SEQ ID NO: 247 - protein, 22 aa, Synthetic construct
MCHQQLVISWFSLVFLASPLVA

SEQ ID NO: 248 - DNA, 66 bp, Synthetic construct
  source 1..66: mol_type = other DNA
atgtgccatcagcaactcgtcatctcctggttctcccttgtgttcctcgcttcccctctggtcgcc

SEQ ID NO: 249 - protein, 14 aa, Synthetic construct
MQLLSCIALILALV

SEQ ID NO: 250 - DNA, 42 bp, Synthetic construct
  source 1..42: mol_type = other DNA
atgcaactgctgtcatgtatcgcactcatcctggcgctggta

SEQ ID NO: 251 - protein, 20 aa, Synthetic construct
MYRMQLLSCIALSLALVTNS

SEQ ID NO: 252 - DNA, 60 bp, Synthetic construct
  source 1..60: mol_type = other DNA
atgtatcggatgcaacttttgagctgcatcgcattgtctctggcgctggtgacaaattcc

SEQ ID NO: 253 - protein, 15 aa, Synthetic construct
MNLLLILTFVAAAVA

SEQ ID NO: 254 - DNA, 45 bp, Synthetic construct
  source 1..45: mol_type = other DNA
atgaatctcttgctcatacttacgtttgtcgctgctgccgttgcg

SEQ ID NO: 255 - protein, 17 aa, Synthetic construct
MGVKVLFALICIAVAEA

SEQ ID NO: 256 - DNA, 51 bp, Synthetic construct
  source 1..51: mol_type = other DNA
atgggcgtgaaggtcttgtttgcccttatctgcatagctgttgcggaggcg

SEQ ID NO: 257 - protein, 24 aa, Synthetic construct
MPMGSLQPLATLYLLGMLVASCLG

SEQ ID NO: 258 - DNA, 72 bp, Synthetic construct
  source 1..72: mol_type = other DNA
atgccgatggggagccttcaacctttggcaacgctttatcttctggggatgttggttgctagttgccttggg

SEQ ID NO: 259 - protein, 21 aa, Synthetic construct
METDTLLLWVLLLWVPGSTGD

SEQ ID NO: 260 - DNA, 63 bp, Synthetic construct
  source 1..63: mol_type = other DNA
atggaaactgacacgttgttgctgtgggtattgctcttgtgggtcccaggatctacgggcgac

SEQ ID NO: 261 - protein, 22 aa, Synthetic construct
MDMRVPAQLLGLLLLWLRGARC

SEQ ID NO: 262 - DNA, 66 bp, Synthetic construct
  source 1..66: mol_type = other DNA
atggatatgagggttcccgcccagcttttggggctgcttttgttgtggcttcgaggggctcggtgt

SEQ ID NO: 263 - protein, 16 aa, Synthetic construct
MKCLLYLAFLFIGVNC

SEQ ID NO: 264 - DNA, 48 bp, Synthetic construct
  source 1..48: mol_type = other DNA
atgaagtgtctgttgtacctggcgtttctgttcattggtgtaaactgt

SEQ ID NO: 265 - protein, 28 aa, Synthetic construct
MNIKGSPWKGSLLLLLVSNLLLCQSVAP

SEQ ID NO: 266 - DNA, 84 bp, Synthetic construct
  source 1..84: mol_type = other DNA
atgaatatcaaaggaagtccgtggaagggtagtctcctgctgctcctcgtatctaaccttctcctttgtcaatccgtggcaccc

SEQ ID NO: 267 - protein, 18 aa, Synthetic construct
MKWVTFISLLFLFSSAYS

SEQ ID NO: 268 - DNA, 54 bp, Synthetic construct
  source 1..54: mol_type = other DNA
atgaaatgggtaacattcatatcacttctctttctgttcagctctgcgtattct

SEQ ID NO: 269 - protein, 19 aa, Synthetic construct
MTRLTVLALLAGLLASSRA

SEQ ID NO: 270 - DNA, 57 bp, Synthetic construct
  source 1..57: mol_type = other DNA
atgacaaggcttactgttttggctctcctcgctggactcttggcttcctcccgagca

SEQ ID NO: 271 - protein, 17 aa, Synthetic construct
MRAWIFFLLCLAGRALA

SEQ ID NO: 272 - DNA, 51 bp, Synthetic construct
  source 1..51: mol_type = other DNA
atgagggcttggattttttttctgctctgccttgccggtcgagccctggcg

SEQ ID NO: 273 - protein, 16 aa, Synthetic construct
MPLLLLLPLLWAGALA

SEQ ID NO: 274 - DNA, 48 bp, Synthetic construct
  source 1..48: mol_type = other DNA
atgcctcttctgcttttgcttcctcttttgtgggcaggtgccctcgca

SEQ ID NO: 275 - protein, 29 aa, Synthetic construct
MNSFSTSAFGPVAFSLGLLLVLPAAFPAP

SEQ ID NO: 276 - DNA, 87 bp, Synthetic construct
  source 1..87: mol_type = other DNA
atgaactctttctcaacctctgcgtttggtccggtcgctttctcccttgggctcctgcttgtcttgccagcagcgtttcctgcgcca

SEQ ID NO: 277 - protein, 20 aa, Synthetic construct
MTSKLAVALLAAFLISAALC

SEQ ID NO: 278 - DNA, 60 bp, Synthetic construct
  source 1..60: mol_type = other DNA
atgacaagtaaactggcggtagccttgctcgcggcctttttgatttccgcagccctttgt

SEQ ID NO: 279 - protein, 23 aa, Synthetic construct
MKVSAALLCLLLIAATFIPQGLA

SEQ ID NO: 280 - DNA, 69 bp, Synthetic construct
  source 1..69: mol_type = other DNA
atgaaggtaagtgcagcgttgctttgccttctcctcattgcagcgacctttattcctcaagggctggcc

SEQ ID NO: 281 - protein, 26 aa, Synthetic construct
MGAAARTLRLALGLLLLATLLRPADA

SEQ ID NO: 282 - DNA, 78 bp, Synthetic construct
  source 1..78: mol_type = other DNA
atgggagcggcagctagaacacttcgacttgcccttgggctcttgctccttgcaaccctccttagacctgccgacgca

SEQ ID NO: 283 - protein, 21 aa, Synthetic construct
MSPLLRRLLLAALLQLAPAQA

SEQ ID NO: 284 - DNA, 63 bp, Synthetic construct
  source 1..63: mol_type = other DNA
atgtcaccgttgttgcggagattgctgttggccgcacttttgcaactggctcctgctcaagcc

SEQ ID NO: 285 - protein, 21 aa, Synthetic construct
MNNLLCCALVFLDISIKWTTQ

SEQ ID NO: 286 - DNA, 63 bp, Synthetic construct
  source 1..63: mol_type = other DNA
atgaataacctgctctgttgtgcgctcgtgttcctggacatttctataaaatggacaacgcaa

SEQ ID NO: 287 - protein, 23 aa, Synthetic construct
MQMSPALTCLVLGLALVFGEGSA

SEQ ID NO: 288 - DNA, 69 bp, Synthetic construct
  source 1..69: mol_type = other DNA
atgcaaatgtctcctgcccttacctgtctcgtacttggtcttgcgctcgtatttggagagggatcagcc

SEQ ID NO: 289 - protein, 34 aa, Synthetic construct
MARAALSAAPSNPRLLRVALLLLLLVAAGRRAAG

SEQ ID NO: 290 - DNA, 102 bp, Synthetic construct
  source 1..102: mol_type = other DNA
atggcaagggctgcactcagtgctgccccgtctaatcccagattgcttcgagttgcattgcttcttctgttgctggttgcagctggtaggagagcagcgggt

SEQ ID NO: 291 - protein, 21 aa, Synthetic construct
MNAKVVVVLVLVLTALCLSDG

SEQ ID NO: 292 - DNA, 63 bp, Synthetic construct
  source 1..63: mol_type = other DNA
atgaatgcaaaagtcgtggtcgtgctggttttggttctgacggcgttgtgtcttagtgatggg

SEQ ID NO: 293 - protein, 22 aa, Synthetic construct
MERIVICLMVIFLGTLVHKSSS

SEQ ID NO: 294 - DNA, 66 bp, Synthetic construct
  source 1..66: mol_type = other DNA
atggaacgcattgtgatctgcctgatggtcatcttcctgggcaccttagtgcacaagtcgagcagc

SEQ ID NO: 295 - protein, 21 aa, Synthetic construct
MALPVTALLLPLALLLHAARP

SEQ ID NO: 296 - DNA, 63 bp, Synthetic construct
  source 1..63: mol_type = other DNA
atggctctccctgtaactgccctgcttcttccccttgccttgcttctccatgccgctagaccc

SEQ ID NO: 297 - protein, 22 aa, Synthetic construct
MLLLVTSLLLCELPHPAFLLIP

SEQ ID NO: 298 - DNA, 66 bp, Synthetic construct
  source 1..66: mol_type = other DNA
atgctgctgctggtcacatctctgctgctgtgcgagctgccccatcctgcctttctgctgatccct

SEQ ID NO: 299 - DNA, 66 bp, Synthetic construct
  source 1..66: mol_type = other DNA
atgctgctgctggttacatctctgctgctgtgcgagctgccccatcctgcctttctgctgatccct

(19) United States
(12) Patent Application Publication
(10) Pub. No.: US 2024/0058384 A1
(43) Pub. Date: Feb. 22, 2024

(54) CHIMERIC ANTIGEN RECEPTORS AND METHODS OF USE

(71) Applicant: SENTI BIOSCIENCES, INC., South San Francisco, CA (US)

(72) Inventor: Marcela Guzman Ayala, South San Francisco, CA (US)

(21) Appl. No.: 18/501,604

(22) Filed: Nov. 3, 2023

Related U.S. Application Data
(63) Continuation of application No. PCT/US2022/028065, filed on May 6, 2022.
(60) Provisional application No. 63/185,391, filed on May 7, 2021.

(51) Int. Cl. A61K 35/17; C07K 16/30; A61P 35/00; A61K 39/00

(52) U.S. Cl. CPC A61K 35/17; C07K 16/303; A61P 35/00; A61K 39/4611; A61K 39/4644; A61K 39/4613; A61K 2239/21; A61K 2239/22; A61K 2239/53; A61K 2239/13

(57) ABSTRACT

The present disclosure provides compositions and methods related to chimeric antigen receptors (CARs). In particular, the present disclosure provides CAR-based immunotherapeutic compositions that target tumor cells expressing glypican-3 (GPC3) for the treatment and prevention of cancer.

### CROSS-REFERENCE TO RELATED APPLICATIONS

This application is a continuation of International Application No. PCT/US2022/028065, filed May 6, 2022, which claims the benefit of U.S. Provisional Patent Application No. 63/185,391, filed May 7, 2021, the contents of which are incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: SENTI-39389.302.xml; Size: 287,793 bytes; and Date of Creation: Nov. 3, 2023.

### FIELD

The present disclosure provides compositions and methods related to chimeric antigen receptors (CARs). In particular, the present disclosure provides CAR-based immunotherapeutic compositions that target tumor cells expressing glypican-3 (GPC3) for the treatment and prevention of cancer.

### BACKGROUND

Chimeric antigen receptors (CARs) are genetically engineered receptors that provide specific properties to an immune effector cell (e.g., a lymphocyte). These receptors gain the specificity of a monoclonal antibody targeted against specific tumor cells. The term “chimeric” indicates different sources of composing parts of the receptor. Lymphocytes with engineered CARs acquire potent immunological properties and by redirecting the immune system in order to eliminate malignant cells act as a living drug, expanding in the patient and ensuring long-term antitumor memory. Current progress in CAR technology includes use in hematological malignancies, solid tumors, the use of dual CAR-T cells and chimeric antigen receptor natural killer cells (CAR-NK cells). For example, liver cancer is the second leading cause of cancer-related deaths worldwide, and hepatocellular carcinoma is the most common type. The pathogenesis of hepatocellular carcinoma is concealed, its progress is rapid, its prognosis is poor, and the mortality rate is high. Therefore, novel molecular targets for hepatocellular carcinoma, early diagnosis, and development of targeted therapy are critically needed. Glypican-3, a cell-surface glycoprotein in which heparan sulfate glycosaminoglycan chains are covalently linked to a protein core, is overexpressed in HCC tissues but not in the healthy adult liver. Thus, Glypican-3 is becoming a promising candidate for liver cancer diagnosis and immunotherapy. Up to now, Glypican-3 has been a reliable immunohistochemical marker for hepatocellular carcinoma diagnosis, and soluble Glypican-3 in serum has become a promising marker for liquid biopsy. Moreover, various immunotherapies targeting Glypican-3 have been developed, including Glypican-3 vaccines, anti-Glypican-3 immunotoxins, and chimeric-antigen-receptor modified cells.

### SUMMARY

Aspects of the present disclosure include a chimeric antigen receptor (CAR) that binds to Glypican-3 (GPC3). In accordance with these aspects, the CAR comprises a single chain Fv (scFv) that binds to GPC3, a transmembrane domain, and one or more intracellular signaling domains, wherein the scFv comprises a heavy chain variable (VH) region and a light chain variable (VL) region pair. In some aspects, the VH and VL pair is selected from the various sequences listed in Table 1. In some aspects, various combinations of CDRs of the VH and VL pairs are selected from the sequences listed in Table 1.

Aspects of the present disclosure include a chimeric antigen receptor (CAR) that binds to Glypican-3 (GPC3), wherein the CAR comprises a single chain Fv (scFv) that binds to GPC3, a transmembrane domain, and one or more intracellular signaling domains, wherein the scFv comprises a heavy chain variable (VH) region and a light chain variable (VL) region pair, and wherein the VH and VL pair is selected from the group consisting of:
a) a VH region comprising a heavy chain complementarity determining region 1 (CDR-H1) having the amino acid sequence of SEQ ID NO: 1, a heavy chain complementarity determining region 2 (CDR-H2) having the amino acid sequence of SEQ ID NO: 2, a heavy chain complementarity determining region 3 (CDR-H3) having the amino acid sequence of SEQ ID NO: 3, and a VL region comprising a light chain complementarity determining region 1 (CDR-L1) having the amino acid sequence of SEQ ID NO: 58, a light chain complementarity determining region 2 (CDR-L2) having the amino acid sequence of SEQ ID NO: 59, and a light chain complementarity determining region 3 (CDR-L3) having the amino acid sequence of SEQ ID NO: 60;
b) a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 5, a heavy chain complementarity determining region 3 (CDR-H3) having the amino acid sequence of SEQ ID NO: 6, and a VL region comprising a light chain complementarity determining region 1 (CDR-L1) having the amino acid sequence of SEQ ID NO: 61, a light chain complementarity determining region 2 (CDR-L2) having the amino acid sequence of SEQ ID NO: 62, and a light chain complementarity determining region 3 (CDR-L3) having the amino acid sequence of SEQ ID NO: 63;
c) a VH region comprising a heavy chain complementarity determining region 1 (CDR-H1) having the amino acid sequence of SEQ ID NO: 7, a heavy chain complementarity determining region 2 (CDR-H2) having the amino acid sequence of SEQ ID NO: 8, a heavy chain complementarity determining region 3 (CDR-H3) having the amino acid sequence of SEQ ID NO: 9, and a VL region comprising a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 66;
d) a VH region comprising a heavy chain complementarity determining region 1 (CDR-H1) having the amino acid sequence of SEQ ID NO: 10, a heavy chain complementarity determining region 2 (CDR-H2) having the amino acid sequence of SEQ ID NO: 11, a heavy chain complementarity determining region 3 (CDR-H3) having the amino acid sequence of SEQ ID NO: 12, and a VL region comprising a light chain complementarity determining region 1 (CDR-L1) having the amino acid sequence of SEQ ID NO: 67, a light chain complementarity determining region 2 (CDR-L2) having the amino acid sequence of SEQ ID NO: 68, and a light chain complementarity determining region 3 (CDR-L3) having the amino acid sequence of SEQ ID NO: 69;
e) a VH region comprising a heavy chain complementarity determining region 1 (CDR-H1) having the amino acid sequence of SEQ ID NO: 13, a heavy chain complementarity determining region 2 (CDR-H2) having the amino acid sequence of SEQ ID NO: 14, a heavy chain complementarity determining region 3 (CDR-H3) having the amino acid sequence of SEQ ID NO: 15, and a VL region comprising a light chain complementarity determining region 1 (CDR-L1) having the amino acid sequence of SEQ ID NO: 70, a light chain complementarity determining region 2 (CDR-L2) having the amino acid sequence of SEQ ID NO: 71, and a light chain complementarity determining region 3 (CDR-L3) having the amino acid sequence of SEQ ID NO: 72;
f) a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 17, a heavy chain complementarity determining region 3 (CDR-H3) having the amino acid sequence of SEQ ID NO: 18, and a VL region comprising a light chain complementarity determining region 1 (CDR-L1) having the amino acid sequence of SEQ ID NO: 73, a light chain complementarity determining region 2 (CDR-L2) having the amino acid sequence of SEQ ID NO: 74, and a light chain complementarity determining region 3 (CDR-L3) having the amino acid sequence of SEQ ID NO: 75;
g) a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 19, a heavy chain complementarity determining region 3 (CDR-H3) having the amino acid sequence of SEQ ID NO: 20, and a VL region comprising a light chain complementarity determining region 1 (CDR-L1) having the amino acid sequence of SEQ ID NO: 76, a light chain complementarity determining region 2 (CDR-L2) having the amino acid sequence of SEQ ID NO: 77, and a light chain complementarity determining region 3 (CDR-L3) having the amino acid sequence of SEQ ID NO: 78;
h) a VH region comprising a heavy chain complementarity determining region 1 (CDR-H1) having the amino acid sequence of SEQ ID NO: 21, a heavy chain complementarity determining region 2 (CDR-H2) having the amino acid sequence of SEQ ID NO: 22, a heavy chain complementarity determining region 3 (CDR-H3) having the amino acid sequence of SEQ ID NO: 23, and a VL region comprising a light chain complementarity determining region 1 (CDR-L1) having the amino acid sequence of SEQ ID NO: 79, a light chain complementarity determining region 2 (CDR-L2) having the amino acid sequence of SEQ ID NO: 80, and a light chain complementarity determining region 3 (CDR-L3) having the amino acid sequence of SEQ ID NO: 81;
i) a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 24, a heavy chain complementarity determining region 3 (CDR-H3) having the amino acid sequence of SEQ ID NO: 25, and a VL region comprising a light chain complementarity determining region 1 (CDR-L1) having the amino acid sequence of SEQ ID NO: 82, a light chain complementarity determining region 2 (CDR-L2) having the amino acid sequence of SEQ ID NO: 65, and a light chain complementarity determining region 3 (CDR-L3) having the amino acid sequence of SEQ ID NO: 83;
j) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
k) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 85, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
l) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 86, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
m) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 87, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
n) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 88, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
o) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 89, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
p) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 90, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
q) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 91, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
r) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 92, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
s) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 93, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
t) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 94, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
u) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 95, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
v) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 96, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
w) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 97, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
x) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 98, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
y) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 99, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
z) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 100, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
aa) a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and a VL region comprising a light chain complementarity determining region 1 (CDR-L1) having the amino acid sequence of SEQ ID NO: 101, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
bb) a VH region comprising a heavy chain complementarity determining region 1 (CDR-H1) having the amino acid sequence of SEQ ID NO: 29, a heavy chain complementarity determining region 2 (CDR-H2) having the amino acid sequence of SEQ ID NO: 30, a heavy chain complementarity determining region 3 (CDR-H3) having the amino acid sequence of SEQ ID NO: 31, and a VL region comprising a light chain complementarity determining region 1 (CDR-L1) having the amino acid sequence of SEQ ID NO: 102, a light chain complementarity determining region 2 (CDR-L2) having the amino acid sequence of SEQ ID NO: 103, and a light chain complementarity determining region 3 (CDR-L3) having the amino acid sequence of SEQ ID NO: 104;
cc) a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and a VL region comprising a light chain complementarity determining region 1 (CDR-L1) having the amino acid sequence of SEQ ID NO: 105, a light chain complementarity determining region 2 (CDR-L2) having the amino acid sequence of SEQ ID NO: 71, and a light chain complementarity determining region 3 (CDR-L3) having the amino acid sequence of SEQ ID NO: 106;
dd) a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and a VL region comprising a light chain complementarity determining region 1 (CDR-L1) having the amino acid sequence of SEQ ID NO: 107, a light chain complementarity determining region 2 (CDR-L2) having the amino acid sequence of SEQ ID NO: 108, and a light chain complementarity determining region 3 (CDR-L3) having the amino acid sequence of SEQ ID NO: 109;
ee) a VH region comprising a heavy chain complementarity determining region 1 (CDR-H1) having the amino acid sequence of SEQ ID NO: 145, a heavy chain complementarity determining region 2 (CDR-H2) having the amino acid sequence of SEQ ID NO: 146, a heavy chain complementarity determining region 3 (CDR-H3) having the amino acid sequence of SEQ ID NO: 147, and a VL region comprising a light chain complementarity determining region 1 (CDR-L1) having the amino acid sequence of SEQ ID NO: 155, a light chain complementarity determining region 2 (CDR-L2) having the amino acid sequence of SEQ ID NO: 156, and a light chain complementarity determining region 3 (CDR-L3) having the amino acid sequence of SEQ ID NO: 157;
ff) a VH region comprising a heavy chain complementarity determining region 1 (CDR-H1) having the amino acid sequence of SEQ ID NO: 148, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 147, and a VL region comprising a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157; and
gg) a VH region comprising a heavy chain complementarity determining region 1 (CDR-H1) having the amino acid sequence of SEQ ID NO: 150, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 151, and a VL region comprising a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157.

In some aspects, the VH region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs: 36, 38, 39, 41, 43, 44, 45, 46, 47, 48, 49, 50, 152, 153, and 154.

In some aspects, the VL region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs: 111, 113, 114, 116, 118-143, and 159-161.

In some aspects, the VH region comprises the amino acid sequence of SEQ ID NO: 36 and the VL region comprises the amino acid sequence of SEQ ID NO: 111.

In some aspects, the VH region comprises the amino acid sequence of SEQ ID NO: 38 and the VL region comprises the amino acid sequence of SEQ ID NO: 113.

In some aspects, the VH region comprises the amino acid sequence of SEQ ID NO: 39 and the VL region comprises the amino acid sequence of SEQ ID NO: 114.

In some aspects, the VH region comprises the amino acid sequence of SEQ ID NO: 41 and the VL region comprises the amino acid sequence of SEQ ID NO: 116.

In some aspects, the VH region comprises the amino acid sequence of SEQ ID NO: 43 and the VL region comprises the amino acid sequence of SEQ ID NO: 118.

In some aspects, the VH region comprises the amino acid sequence of SEQ ID NO: 44 and the VL region comprises the amino acid sequence of SEQ ID NO: 119.

In some aspects, the VH region comprises the amino acid sequence of SEQ ID NO: 45 and the VL region comprises the amino acid sequence of SEQ ID NO: 120.

In some aspects, the VH region comprises the amino acid sequence of SEQ ID NO: 46 and the VL region comprises the amino acid sequence of SEQ ID NO: 121.

In some aspects, the VH region comprises the amino acid sequence of SEQ ID NO: 47 and the VL region comprises the amino acid sequence of SEQ ID NO: 122.

In some aspects, the VH region comprises the amino acid sequence of SEQ ID NO: 48 and the VL region comprises the amino acid sequence of SEQ ID NO: 123.

In some aspects, the VH region comprises the amino acid sequence of SEQ ID NO: 48 and the VL region comprises the amino acid sequence of SEQ ID NO: 124.

In some aspects, the VH region comprises the amino acid sequence of SEQ ID NO: 48 and the VL region comprises the amino acid sequence of SEQ ID NO: 125.

In some aspects, the VH region comprises the amino acid sequence of SEQ ID NO: 48 and the VL region comprises the amino acid sequence of SEQ ID NO: 126.

In some aspects, the VH region comprises the amino acid sequence of SEQ ID NO: 48 and the VL region comprises the amino acid sequence of SEQ ID NO: 127.

In some aspects, the VH region comprises the amino acid sequence of SEQ ID NO: 48 and the VL region comprises the amino acid sequence of SEQ ID NO: 128.

In some aspects, the VH region comprises the amino acid sequence of SEQ ID NO: 48 and the VL region comprises the amino acid sequence of SEQ ID NO: 129.

In some aspects, the VH region comprises the amino acid sequence of SEQ ID NO: 48 and the VL region comprises the amino acid sequence of SEQ ID NO: 130.

In some aspects, the VH region comprises the amino acid sequence of SEQ ID NO: 48 and the VL region comprises the amino acid sequence of SEQ ID NO: 131.

In some aspects, the VH region comprises the amino acid sequence of SEQ ID NO: 48 and the VL region comprises the amino acid sequence of SEQ ID NO: 132.

In some aspects, the VH region comprises the amino acid sequence of SEQ ID NO: 48 and the VL region comprises the amino acid sequence of SEQ ID NO: 133.

In some aspects, the VH region comprises the amino acid sequence of SEQ ID NO: 48 and the VL region comprises the amino acid sequence of SEQ ID NO: 134.

In some aspects, the VH region comprises the amino acid sequence of SEQ ID NO: 48 and the VL region comprises the amino acid sequence of SEQ ID NO: 135.

In some aspects, the VH region comprises the amino acid sequence of SEQ ID NO: 48 and the VL region comprises the amino acid sequence of SEQ ID NO: 136.

In some aspects, the VH region comprises the amino acid sequence of SEQ ID NO: 48 and the VL region comprises the amino acid sequence of SEQ ID NO: 137.

In some aspects, the VH region comprises the amino acid sequence of SEQ ID NO: 48 and the VL region comprises the amino acid sequence of SEQ ID NO: 138.

In some aspects, the VH region comprises the amino acid sequence of SEQ ID NO: 48 and the VL region comprises the amino acid sequence of SEQ ID NO: 139.

In some aspects, the VH region comprises the amino acid sequence of SEQ ID NO: 48 and the VL region comprises the amino acid sequence of SEQ ID NO: 140.

In some aspects, the VH region comprises the amino acid sequence of SEQ ID NO: 49 and the VL region comprises the amino acid sequence of SEQ ID NO: 141.

In some aspects, the VH region comprises the amino acid sequence of SEQ ID NO: 50 and the VL region comprises the amino acid sequence of SEQ ID NO: 142.

In some aspects, the VH region comprises the amino acid sequence of SEQ ID NO: 50 and the VL region comprises the amino acid sequence of SEQ ID NO: 143.

In some aspects, the VH region comprises the amino acid sequence of SEQ ID NO: 152 and the VL region comprises the amino acid sequence of SEQ ID NO: 159.

In some aspects, the VH region comprises the amino acid sequence of SEQ ID NO: 153 and the VL region comprises the amino acid sequence of SEQ ID NO: 160.

In some aspects, the VH region comprises the amino acid sequence of SEQ ID NO: 154 and the VL region comprises the amino acid sequence of SEQ ID NO: 161.

Aspects of the present disclosure include a chimeric antigen receptor (CAR) that binds to Glypican-3 (GPC3), wherein the CAR comprises a single chain Fv (scFv) that binds to GPC3, a transmembrane domain, and one or more intracellular signaling domains, wherein the scFv comprises a heavy chain variable (VH) region and a light chain variable (VL) region, wherein the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of an amino acid sequence selected from the group consisting of SEQ ID NO: 35-57 and 152-154; and wherein the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of an amino acid sequence selected from the group consisting of SEQ ID NO: 110-144 and 159-161.

Aspects of the present disclosure include a chimeric antigen receptor (CAR) that binds to Glypican-3 (GPC3), wherein the CAR comprises a single chain Fv (scFv) that binds to GPC3, a transmembrane domain, and one or more intracellular signaling domains, wherein the scFv comprises a heavy chain variable (VH) region and a light chain variable (VL) region, wherein the VH comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 35-57 and 152-154; and wherein the VL comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 110-144 and 159-161.

Aspects of the present disclosure include a chimeric antigen receptor (CAR) that binds to Glypican-3 (GPC3), wherein the CAR comprises a single chain Fv (scFv) that binds to GPC3, a transmembrane domain, and one or more intracellular signaling domains, wherein the scFv comprises a heavy chain variable (VH) region and a light chain variable (VL) region pair, and wherein the VH and VL pair is selected from the group consisting of:
a) a VH region having the amino acid sequence of SEQ ID NO: 35 and VL region having the amino acid sequence of SEQ ID NO: 110;
b) a VH region having the amino acid sequence of SEQ ID NO: 36 and VL region having the amino acid sequence of SEQ ID NO: 111;
c) a VH region having the amino acid sequence of SEQ ID NO: 37 and VL region having the amino acid sequence of SEQ ID NO: 112;
d) a VH region having the amino acid sequence of SEQ ID NO: 38 and VL region having the amino acid sequence of SEQ ID NO: 113;
e) a VH region having the amino acid sequence of SEQ ID NO: 39 and VL region having the amino acid sequence of SEQ ID NO: 114;
f) a VH region having the amino acid sequence of SEQ ID NO: 40 and VL region having the amino acid sequence of SEQ ID NO: 115;
g) a VH region having the amino acid sequence of SEQ ID NO: 41 and VL region having the amino acid sequence of SEQ ID NO: 116;
h) a VH region having the amino acid sequence of SEQ ID NO: 42 and VL region having the amino acid sequence of SEQ ID NO: 117;
i) a VH region having the amino acid sequence of SEQ ID NO: 43 and VL region having the amino acid sequence of SEQ ID NO: 118;
j) a VH region having the amino acid sequence of SEQ ID NO: 44 and VL region having the amino acid sequence of SEQ ID NO: 119;
k) a VH region having the amino acid sequence of SEQ ID NO: 45 and VL region having the amino acid sequence of SEQ ID NO: 120;
l) a VH region having the amino acid sequence of SEQ ID NO: 46 and VL region having the amino acid sequence of SEQ ID NO: 121;
m) a VH region having the amino acid sequence of SEQ ID NO: 47 and VL region having the amino acid sequence of SEQ ID NO: 122;
n) a VH region having the amino acid sequence of SEQ ID NO: 48 and VL region having the amino acid sequence of SEQ ID NO: 123;
o) a VH region having the amino acid sequence of SEQ ID NO: 48 and VL region having the amino acid sequence of SEQ ID NO: 124;
p) a VH region having the amino acid sequence of SEQ ID NO: 48 and VL region having the amino acid sequence of SEQ ID NO: 125;
q) a VH region having the amino acid sequence of SEQ ID NO: 48 and VL region having the amino acid sequence of SEQ ID NO: 126;
r) a VH region having the amino acid sequence of SEQ ID NO: 48 and VL region having the amino acid sequence of SEQ ID NO: 127;
s) a VH region having the amino acid sequence of SEQ ID NO: 48 and VL region having the amino acid sequence of SEQ ID NO: 128;
t) a VH region having the amino acid sequence of SEQ ID NO: 48 and VL region having the amino acid sequence of SEQ ID NO: 129;
u) a VH region having the amino acid sequence of SEQ ID NO: 48 and VL region having the amino acid sequence of SEQ ID NO: 130;
v) a VH region having the amino acid sequence of SEQ ID NO: 48 and VL region having the amino acid sequence of SEQ ID NO: 131;
w) a VH region having the amino acid sequence of SEQ ID NO: 48 and VL region having the amino acid sequence of SEQ ID NO: 132;
x) a VH region having the amino acid sequence of SEQ ID NO: 48 and VL region having the amino acid sequence of SEQ ID NO: 133;
y) a VH region having the amino acid sequence of SEQ ID NO: 48 and VL region having the amino acid sequence of SEQ ID NO: 134;
z) a VH region having the amino acid sequence of SEQ ID NO: 48 and VL region having the amino acid sequence of SEQ ID NO: 135;
aa) a VH region having the amino acid sequence of SEQ ID NO: 48 and VL region having the amino acid sequence of SEQ ID NO: 136;
bb) a VH region having the amino acid sequence of SEQ ID NO: 48 and VL region having the amino acid sequence of SEQ ID NO: 137;
cc) a VH region having the amino acid sequence of SEQ ID NO: 48 and VL region having the amino acid sequence of SEQ ID NO: 138;
dd) a VH region having the amino acid sequence of SEQ ID NO: 48 and VL region having the amino acid sequence of SEQ ID NO: 139;
ee) a VH region having the amino acid sequence of SEQ ID NO: 48 and VL region having the amino acid sequence of SEQ ID NO: 140;
ff) a VH region having the amino acid sequence of SEQ ID NO: 49 and VL region having the amino acid sequence of SEQ ID NO: 141;
gg) a VH region having the amino acid sequence of SEQ ID NO: 50 and VL region having the amino acid sequence of SEQ ID NO: 142;
hh) a VH region having the amino acid sequence of SEQ ID NO: 50 and VL region having the amino acid sequence of SEQ ID NO: 143;
ii) a VH region having the amino acid sequence of SEQ ID NO: 51 and VL region having the amino acid sequence of SEQ ID NO: 144;
jj) a VH region having the amino acid sequence of SEQ ID NO: 52 and VL region having the amino acid sequence of SEQ ID NO: 144;
kk) a VH region having the amino acid sequence of SEQ ID NO: 53 and VL region having the amino acid sequence of SEQ ID NO: 144;
ll) a VH region having the amino acid sequence of SEQ ID NO: 54 and VL region having the amino acid sequence of SEQ ID NO: 144;
mm) a VH region having the amino acid sequence of SEQ ID NO: 55 and VL region having the amino acid sequence of SEQ ID NO: 144;
nn) a VH region having the amino acid sequence of SEQ ID NO: 56 and VL region having the amino acid sequence of SEQ ID NO: 144;
oo) a VH region having the amino acid sequence of SEQ ID NO: 57 and VL region having the amino acid sequence of SEQ ID NO: 144;
pp) a VH region having the amino acid sequence of SEQ ID NO: 152 and VL region having the amino acid sequence of SEQ ID NO: 159;
qq) a VH region having the amino acid sequence of SEQ ID NO: 153 and VL region having the amino acid sequence of SEQ ID NO: 160; and
rr) a VH region having the amino acid sequence of SEQ ID NO: 154 and VL region having the amino acid sequence of SEQ ID NO: 161.

In some aspects, the VH and VL of the scFv are separated by a peptide linker.

In some aspects, the scFV comprises the structure VH-L-VL or VL-L-VH, wherein VH is the heavy chain variable region, L is the peptide linker, and VL is the light chain variable region.

In some aspects, the peptide linker comprises an amino acid sequence selected from the group consisting of SEQ ID No: 162-180.

In some aspects, the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2 DS1 transmembrane domain, a KIR3 DS1 transmembrane domain, an NKp44 transmembrane domain, an NKp46 transmembrane domain, an FceRlg transmembrane domain, and an NKG2D transmembrane domain.

In some aspects, the one or more intracellular signaling domains are each selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2 DS1 intracellular signaling domain, a KIR3 DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

In some aspects, the CAR comprises one or more of a hinge domain, a spacer region, or one or more peptide linkers.

In some aspects, the CAR comprises a spacer region between the scFV and the transmembrane domain.

In some aspects, the spacer region has an amino acid sequence selected from the group consisting of SEQ ID NOs: 181-90.

Aspects of the disclosure also include compositions comprising a CAR as described herein, and a pharmaceutically acceptable carrier, pharmaceutically acceptable excipient, or a combination thereof.

Aspects of the present disclosure also include an engineered nucleic acid encoding a CAR as described herein.

Aspects of the present disclosure also include an expression vector comprising an engineered nucleic acid as described herein.

Aspects of the present disclosure also include compositions comprising an engineered nucleic acid as described herein or an expression vector as described herein, and a pharmaceutically acceptable carrier, pharmaceutically acceptable excipient, or a combination thereof.

Aspects of the present disclosure also include a method of making an engineered cell, comprising transducing an isolated cell with an engineered nucleic acid as described herein or an expression vector as described herein.

Aspects of the present disclosure also include an isolated cell or a population of cells comprising an engineered nucleic acid or an expression vector as described herein.

Aspects of the present disclosure also include an isolated cell or a population of cells expressing an engineered nucleic acid or an expression vector as described herein.

Aspects of the present disclosure also include an isolated cell comprising a CAR as described herein.

Aspects of the present disclosure also include a population of cells comprising a CAR as described herein.

In some aspects, the CAR is recombinantly expressed by the cell or population of cells.

In some aspects, the CAR is expressed from a vector or a selected locus from the genome of the cell.

In some aspects, the cell or population of cells further expresses one or more immunomodulating effectors.

In some aspects, the one or more immunomodulating effectors are one or more cytokines or chemokines.

In some aspects, the one or more cytokines or chemokines are selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, TNF-alpha, CCL21a, CXCL10, CXCL11, CXCL13, a CXCL10-CXCL11 fusion protein, CCL19, CXCL9, and XCL1.

In some aspects, expression of the one or more immunomodulating effectors is controlled by an activation-conditional control polypeptide (ACP).

In some aspects, the one or more immunomodulating effectors are expressed from one or more expression cassettes, wherein the one or more expression cassettes each comprises an ACP-responsive promoter and an exogenous polynucleotide sequence encoding one or more immunomodulating effectors, wherein the ACP-responsive promoter is operably linked to the exogenous polynucleotide.

In some aspects, the ACP is capable of inducing expression of the one or more expression cassettes by binding to the ACP-responsive promoter. In some aspects, the ACP-responsive promoter comprises an ACP-binding domain and a promoter sequence.

In some aspects, the promoter sequence is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB_TATA, minTATA, minTK, inducer molecule responsive promoters, and tandem repeats thereof. In some aspects, the ACP-responsive promoter is a synthetic promoter. In some aspects, the ACP-responsive promoter comprises a minimal promoter.

In some aspects, the ACP-binding domain comprises one or more zinc finger binding sites.

In some aspects, the ACP is a transcriptional modulator. In some aspects, the ACP is a transcriptional repressor. In some aspects, the ACP is a transcriptional activator.

In some aspects, the ACP further comprises a repressible protease and one or more cognate cleavage sites of the repressible protease.

In some aspects, the ACP further comprises a hormone-binding domain of estrogen receptor (ERT2 domain).

In some aspects, the ACP is a transcription factor. In some aspects, the ACP is a zinc-finger-containing transcription factor.

In some aspects, the zinc finger-containing transcription factor comprises a DNA-binding zinc finger protein domain (ZF protein domain) and an effector domain.

In some aspects, the ZF protein domain is modular in design and is composed of zinc finger arrays (ZFA). In some aspects, the ZF protein domain comprises one to ten ZFA.

In some aspects, the effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSP90 activation domains (VPH activation domain); a histone acetyltransferase (HAT) core domain of the human E1A-associated protein p300 (p300 HAT core activation domain); a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the one or more cognate cleavage sites of the repressible protease are localized between the ZF protein domain and the effector domain.

In some aspects, the repressible protease is a hepatitis C virus (HCV) nonstructural protein 3 (NS3).

In some aspects, the cognate cleavage site comprises an NS3 protease cleavage site. In some aspects, the NS3 protease cleavage site comprises a NS3/NS4A, a NS4A/NS4B, a NS4B/NS5A, or a NS5A/NS5B junction cleavage site. In some aspects, the NS3 protease can be repressed by a protease inhibitor.

In some aspects, the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir.

In some aspects, the protease inhibitor comprises grazoprevir.

In some aspects, the ACP is capable of undergoing nuclear localization upon binding of the ERT2 domain to tamoxifen or a metabolite thereof.

In some aspects, the tamoxifen metabolite is selected from the group consisting of: 4-hydroxytamoxifen, N-desmethyltamoxifen, tamoxifen-N-oxide, and endoxifen.

In some aspects, the ACP further comprises a degron, and wherein the degron is operably linked to the ACP. In some aspects, the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of omithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

In some aspects, the degron comprises a cereblon (CRBN) polypeptide substrate domain capable of binding CRBN in response to an immunomodulatory drug (IMiD) thereby promoting ubiquitin pathway-mediated degradation of the ACP. In some aspects, the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

In some aspects, the CRBN polypeptide substrate domain is a chimeric fusion product of native CRBN polypeptide sequences. In some aspects, the CRBN polypeptide substrate domain is a IKZF3/ZFP91/IKZF3 chimeric fusion product having the amino acid sequence of FNVLM VHKRS HTGER PLQCE ICGFT CRQKG NLLRH IKLHT GEKPF KCHLC NYACQ RRDAL.

In some aspects, the IMiD is an FDA-approved drug. In some aspects, the IMiD is selected from the group consisting of: thalidomide, lenalidomide, and pomalidomide.

In some aspects, the degron is localized 5′ of the repressible protease, 3′ of the repressible protease, 5′ of the ZF protein domain, 3′ of the ZF protein domain, 5′ of the effector domain, or 3′ of the effector domain.

In some aspects, the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In some aspects, the cell or population of cells is a Natural Killer (NK) cell.

In some aspects, the cell or population of cells is autologous. In some aspects, the cell or population of cells is allogeneic.

Aspects of the present disclosure also include a pharmaceutical composition comprising an effective amount of the cell or population of engineered cells as described herein and a pharmaceutically acceptable carrier, pharmaceutically acceptable excipient, or a combination thereof.

Aspects of the present disclosure also include a pharmaceutical composition comprising an effective amount of genetically modified cells expressing a CAR as described herein and a pharmaceutically acceptable carrier, pharmaceutically acceptable excipient, or a combination thereof.

In some aspects, the pharmaceutical composition is for treating and/or preventing a tumor.

Aspects of the present disclosure also include a method of treating a subject in need thereof, the method comprising administering a therapeutically effective dose of a composition as described herein or an isolated cell as described herein.

Aspects of the present disclosure also include method of stimulating a cell-mediated immune response to a tumor cell in a subject, the method comprising administering to a subject having a tumor a therapeutically effective dose of a composition as described herein or an isolated cell as described herein.

Aspects of the present disclosure also include a method of treating a subject having a tumor, the method comprising administering a therapeutically effective dose of a composition as described herein or an isolated cell as described herein.

Aspects of the present disclosure also include kits for treating/and preventing a tumor.

In some aspects, the kit comprises a CAR as described herein. In some aspects, the kit further comprises written instructions for using the chimeric protein for producing one or more antigen-specific cells for treating and/or preventing a tumor in a subject.

In some aspects, the kit comprises a cell or population of cells as described herein. In some aspects, the kit further comprises written instructions for using the cell for treating and/or preventing a tumor in a subject.

In some aspects, the kit comprises an isolated nucleic acid as described herein. In some aspects, the kit further comprises written instructions for using the nucleic acid for producing one or more antigen-specific cells for treating and/or preventing a tumor in a subject.

In some aspects, the kit comprises a vector as described herein. In some aspects, the kit further comprises written instructions for using the vector for producing one or more antigen-specific cells for treating and/or preventing a tumor in a subject.

In some aspects, the kit comprises a composition as described herein. In some aspects, the kit further comprises written instructions for using the composition for treating and/or preventing a tumor in a subject.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1: Representative data from flow cytometry experiments demonstrating expression of a GPC3-specific CAR in T cells, according to one embodiment of the present disclosure.

FIGS. 2A-2D: Representative data from functional assays demonstrating effective targeting and killing of GPC3-expressing liver tumor cells (HepG2 and Hep3B) by the GPC3-specific CAR T-cells of the present disclosure (FIGS. 2A and 2C); additional representative data demonstrating expression of various cytokines (IL-2, INFγ, and TNFα) in these cells (FIGS. 2B and 2D).

FIGS. 3A-3D: Representative data demonstrating the in vivo efficacy of the GPC3-specific CAR T-cells of the present disclosure against GPC3-expressing liver tumor cells (HepG2 and Hep3B); GPC3-specific CAR T-cells decreased the number of tumor cells present in mice post-injection (FIGS. 3A and 3B) and increased their overall survival (FIGS. 3C and 3D).

FIG. 4: Representative data from flow cytometry experiments demonstrating expression of a GPC3-specific CAR in NK cells, according to one embodiment of the present disclosure.

FIGS. 5A-5D: Representative data from functional assays demonstrating percent killing of GPC3-expressing liver tumor cells (HepG2 and Huh7) by the GPC3-specific CAR NK-cells of the present disclosure (FIGS. 5A and 5C); additional representative data demonstrating expression of various cytokines (INFγ, TNFα, GrnzB) in these cells (FIGS. 5B and 5D).

### DETAILED DESCRIPTION

### Definitions

Terms used in the claims and specification are defined as set forth below unless otherwise specified.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov).

As provided herein, “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Engineered Nucleic Acids and Polypeptides

Embodiments of the present disclosure include chimeric antigen receptors (CARs) that target cells (e.g., tumor cells) expressing Glypican-3 (GPC3), referred to herein as GPC3 CARs, as well as nucleic acid molecules encoding GPC3 CARs. The GPC3 CAR polypeptides and polynucleotides of the present disclosure include an extracellular portion comprising an antigen binding domain specific for a GPC3 antigen, a transmembrane domain, and one or more intracellular signaling domains. In some embodiments, the GPC3 CARs include one or more of a hinge domain, a spacer region, and/or one or more peptide linkers. When engineered to be expressed on the surface of an immune cell (e.g., T lymphocyte, NK cell), the GPC3 CARs of the present disclosure target GPC3-expressing cells (e.g., tumor cells), which results in the targeted destruction of those cells.

GPC3 Binding Domains

Certain aspects of the present disclosure relate to a chimeric antigen receptor (CAR) that includes a Glypican-3 (GPC3) binding domain. In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, the scFv comprises a heavy chain variable domain (VH) and a light chain variable domain (VL). In some embodiments, the VH and VL are separated by a peptide linker. Generally, an scFv has a variable domain of light chain (VL) connected from its C-terminus to the N-terminal end of a variable domain of heavy chain (VH) by a polypeptide chain. Alternately, the scFv comprises of polypeptide chain where in the C-terminal end of the VH is connected to the N-terminal end ofVL by a polypeptide chain. In some embodiments, the scFv comprises the structure VH-L-VL or VL-L-VH, wherein VH is the heavy chain variable domain, L is the peptide linker, and VL is the light chain variable domain. An sdAb is a molecule in which one variable domain of an antibody specifically binds to an antigen without the presence of the other variable domain. A F(ab) fragment contains the constant domain (CL) of the light chain and the first constant domain (CHI) of the heavy chain along with the variable domains VL and VH on the light and heavy chains respectively. F(ab′) fragments differ from Fab fragments by the addition of a few residues at the carboxy terminus of the heavy chain CHI domain including one or more cysteines from the antibody hinge region. F(ab′)₂ fragments contain two Fab′ fragments joined, near the hinge region, by disulfide bonds.

In some embodiments, the present disclosure provides a chimeric antigen receptor (CAR) comprising a single chain variable fragment (scFv) that binds to GPC3, wherein the scFv comprises a heavy chain variable (VH) region and a light chain variable (VL) region pair.

In some embodiments, the VH and VL pairs of the GPC3 CARs of the present are selected from the various sequences listed in Table 1. In some embodiments, various combinations of CDRs of the VH and VL pairs are selected from the sequences listed in Table 1. The various embodiments of the present disclosure may include one or more of the polypeptide sequences pertaining to GPC3 antibodies referenced below in Table 1.

TABLE 1
GPC3 antibody amino acid sequences.

| Antibody | Antibody | | SEQ |
|---|---|---|---|
| Clone(s) | Feature | Sequence | ID NO: |
| M13B3 | CDR-H1 | NYAMS | 1 |
| | CDR-H2 | AINNNGDDTYYLDTVKD | 2 |
| | CDR-H3 | QGGAY | 3 |
| M3B8 | CDR-H1 | TYGMGVG | 4 |
| GC202 | CDR-H2 | NIWWYDAKYYNSDLKS | 5 |
| M3B8 | CDR-H3 | MGLAWFAY | 6 |
| M11F1 | CDR-H1 | IYGMGVG | 7 |
| | CDR-H2 | NIWWNDDKYYNSALKS | 8 |
| | CDR-H3 | IGYFYFDY | 9 |
| M6B1 | CDR-H1 | SYAMS | 10 |
| | CDR-H2 | AINSNGGTTYYPDTMKD | 11 |
| | CDR-H3 | HNGGYENYGWFAY | 12 |
| M5B9 | CDR-H1 | GYWMH | 13 |
| | CDR-H2 | AIYPGNSDTNYNQKFKG | 14 |
| | CDR-H3 | SGDLTGGLAY | 15 |
| M10D2 | CDR-H1 | SYWMH | 16 |
| L9G11 | | | |
| M10D2 | CDR-H2 | EIDPSDSYTYYNQKFRG | 17 |
| | CDR-H3 | SNLGDGHYRFPAFPY | 18 |
| L9G11 | CDR-H2 | TIDPSDSETHYNLQFKD | 19 |
| | CDR-H3 | GAFYSSYSYWAWFAY | 20 |
| GC199 | CDR-H1 | DYSMH | 21 |
| | CDR-H2 | WINTETGEPTYADDFKG | 22 |
| | CDR-H3 | LY | 23 |
| GC202 | CDR-H2 | NIWWHDDKYYNSALKS | 24 |
| | CDR-H3 | IAPRYNKYEGFFAF | 25 |
| GC33 | CDR-H1 | DYEMH | 26 |
| GC33G34A | CDR-H2 | ALDPKTGDTAYSQKFKG | 27 |
| GC33G34D | CDR-H3 | FYSYTY | 28 |
| GC33G34E | CDR-H3 | | |
| GC33G34F | CDR-H3 | | |
| GC33G34H | CDR-H3 | | |
| GC33G34N | CDR-H3 | | |
| GC33G34T | CDR-H3 | | |
| GC33G34Q | CDR-H3 | | |
| GC33G34I | CDR-H3 | | |
| GC33G34K | CDR-H3 | | |
| GC33G34L | CDR-H3 | | |
| GC33G34S | CDR-H3 | | |
| GC33G34W | CDR-H3 | | |
| GC33G34Y | CDR-H3 | | |
| GC33G34R | CDR-H3 | | |
| GC33G34V | CDR-H3 | | |
| GC33G34P | CDR-H3 | | |
| GC179 | CDR-H1 | INAMN | 29 |
| | CDR-H2 | RIRSESNNYATYYGDSVKD | 30 |
| | CDR-H3 | EVTTSFAY | 31 |
| GC194 (1) | CDR-H1 | ASAMN | 32 |
| GC194 (2) | CDR-H2 | RIRSKSNNYAIYYADSVKD | 33 |
| | CDR-H3 | DPGYYGNPWFAY | 34 |
| MC311 | HCVR | MNFGLTLIFLVLTLKGVQCEVQLVESGGGLVKPGGSLKLSCA | 35 |
| | | ASGFTFSRYAMSWVRQIPEKILEWVAAIDSSGGDTYYLDTVK | |
| | | DRFTISRDNANNTLHLQMRSLRSEDTALYYCVRQGGAYWGQ | |
| | | GTLVTVSAASTKGPSVFPLAPSSKSTSGGTAALGCLVKDYFPE | |
| | | PVTVSWNSGALTSGVHTFPAVLQSSGLYSLSSVVTVPSSSLGT | |
| | | QTYICNVNHKPSNTKVDKKVEPKSCDKTHTCPPCPAPELLGGP | |
| | | SVFLFPPKPKDTLMISRTPEVTCVVVDVSHEDPEVKFNWYVD | |
| | | GVEVHNAKTKPREEQYNSTYRVVSVLTVLHQDWLNGKEYKC | |
| | | KVSNKALPAPIEKTISKAKGQPREPQVYTLPPSRDELTKNQVSL | |
| | | TCLVKGFYPSDIAVEWESNGQPENNYKTTPPVLDSDGSFFLYS | |
| | | KLTVDKSRWQQGNVFSCSVMHEALHNHYTQKSLSLSPGK | |
| M13B3 | HCVR | EVHLVESGGGLVKPGGSLKLSCAASGFTFSNYAMSWVRQTPE | 36 |
| | | KRLEWVAAINNNGDDTYYLDTVKDRFTISRDNAKNTLYLQM | |
| | | SSLRSEDTALYYCVRQGGAYWGQGTLVTVSA | |
| M1E7 | HCVR | MGWNWIFILILSVTTGVHSEVQLQQSGPELVKPGASVKISCKA | 37 |
| | | SGYSFTGYYMHWVKQSPEKSLEWIGEINPSTGGTTYNQKFKA | |
| | | KATLTVDKSSSTAYMQLKSLTSEDSAVYYCARRGGLTGTSFF | |
| | | AYWGQGTLVTVSAASTKGPSVFPLAPSSKSTSGGTAALGCLV | |
| | | KDYFPEPVTVSWNSGALTSGVHTFPAVLQSSGLYSLSSVVTVP | |
| | | SSSLGTQTYICNVNHKPSNTKVDKKVEPKSCDKTHTCPPCPAP | |
| | | ELLGGPSVFLFPPKPKDTLMISRTPEVTCVVVDVSHEDPEVKF | |
| | | NWYVDGVEVHNAKTKPREEQYNSTYRVVSVLTVLHQDWLN | |
| | | GKEYKCKVSNKALPAPIEKTISKAKGQPREPQVYTLPPSRDEL | |
| | | TKNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGK | |
| M3B8 | HCVR | QVTLKESGPGILQPSQTLSLTCSFSGFSLSTYGMGVGWIRQPSG | 38 |
| | | MGLEWLANIWWYDAKYYNSDLKSRLTISKDTSNNQVFLKISS | |
| | | VDTSDTATYYCAQMGLAWFAYWGQGTLVTVSA | |
| M11F1 | HCVR | QVTLKESGPGILQPSQTLSLTCSFSGFSLSIYGMGVGWIRQPSG | 39 |
| | | KGLEWLANIWWNDDKYYNSALKSRLTISKDTSNNQVFLKISS | |
| | | VDTADTATYYCAQIGYFYFDYWGQGTTLTVSS | |
| M19B11 | HCVR | MNFGLTLIFLVLTLKGVQCEVQLVESGGDLVKPGGTLKLSCA | 40 |
| | | ASGSTFSNYAMSWVRQTPEKRLEWVAAIDSNGGTTYYPDTM | |
| | | KDRFTISRDNAKNTLYLQMNSLRSEDTAFYHCTRHNGGYENY | |
| | | GWFAYWGQGTLVTVSAASTKGPSVFPLAPSSKSTSGGTAALG | |
| | | CLVKDYFPEPVTVSWNSGALTSGVHTFPAVLQSSGLYSLSSVV | |
| | | TVPSSSLGTQTYICNVNHKPSNTKVDKKVEPKSCDKTHTCPPC | |
| | | PAPELLGGPSVFLFPPKPKDTLMISRTPEVTCVVVDVSHEDPEV | |
| | | KFNWYVDGVEVHNAKTKPREEQYNSTYRVVSVLTVLHQDW | |
| | | LNGKEYKCKVSNKALPAPIEKTISKAKGQPREPQVYTLPPSRD | |
| | | ELTKNQVSLTCLVKGFYPSDIAVEWESNGQPENNYKTTPPVL | |
| | | DSDGSFFLYSKLTVDKSRWQQGNVFSCSVMHEALHNHYTQK | |
| | | SLSLSPGK | |
| M6B1 | HCVR | EVQLVESGGDLVKPGGSLKLSCAASGFTFSSYAMSWVRQTPE | 41 |
| | | KRLEWVAAINSNGGTTYYPDTMKDRFTISRDNAKNTLYLQMS | |
| | | SLRSEDSALYYCTRHNGGYENYGWFAYWGQGTLVTVSA | |
| M18D4 | HCVR | MESNWILPFILSVASGVYSEVQLQQSGTVLARPGASVKMSCK | 42 |
| | | ASGYTFTGYWMRWVKQRPGQGLEWIGAIYPGNSDTTYNQKF | |
| | | KGKAKLTAVTSVSTAYMELSSLTNEDSAVYYCSRSGDLTGGF | |
| | | AYWGQGTLVTVSTAKASTKGPSVFPLAPSSKSTSGGTAALGC | |
| | | LVKDYFPEPVTVSWNSGALTSGVHTFPAVLQSSGLYSLSSVVT | |
| | | VPSSSLGTQTYICNVNHKPSNTKVDKKVEPKSCDKTHTCPPCP | |
| | | APELLGGPSVFLFPPKPKDTLMISRTPEVTCVVVDVSHEDPEV | |
| | | KFNWYVDGVEVHNAKTKPREEQYNSTYRVVSVLTVLHQDW | |
| | | LNGKEYKCKVSNKALPAPIEKTISKAKGQPREPQVYTLPPSRD | |
| | | ELTKNQVSLTCLVKGFYPSDIAVEWESNGQPENNYKTTPPVL | |
| | | DSDGSFFLYSKLTVDKSRWQQGNVFSCSVMHEALHNHYTQK | |
| | | SLSLSPGK | |
| M5B9 | HCVR | EVQLQQSGTVLARPGASVKMSCKASGYTFTGYWMHWVKQR | 43 |
| | | PGQGLEWIGAIYPGNSDTNYNQKFKGKAKLTAVTSASTAYME | |
| | | LSSLTNEDAAVYHCTRSGDLTGGLAYWGQGTLVTVSA | |
| M10D2 | HCVR | QVQLQQPGAELVKPGASVKLSCKASGYTFTSYWMHWVKQRP | 44 |
| | | GQGLEWIGEIDPSDSYTYYNQKFRGKATLTVDKSSNTAYMQL | |
| | | SSLTSEDSAVYYCSRSNLGDGHYRFPAFPYWGQGTLVTVSA | |
| L9G11 | HCVR | QVQLQQPGAELVKPGASVKLSCKASGYTFTSYWMHWVKQRP | 45 |
| | | GQGLEWIGTIDPSDSETHYNLQFKDTATLTVDKSSSTAYMQLS | |
| | | SLTSEDSAVYYCIRGAFYSSYSYWAWFAYWGQGTLVTVSA | |
| GC199 | HCVR | QIQLEQSGPELKKPGETVKISCKASGYIFRDYSMHWVKQAPG | 46 |
| | | KGLKWMGWINTETGEPTYADDFKGRFAFSLETSASTAYLQIN | |
| | | NLKNEDTATYFCTSLYWGQGTLVTVSA | |
| GC202 | HCVR | QVTLKESGPGILQPSQTLSLTCSFSGFSLSTYGMGVGWIRQPSG | 47 |
| | | KGLEWLANIWWHDDKYYNSALKSRLTISKDISNNQVFLKISS | |
| | | VDTADTATYYCAQIAPRYNKYEGFFAFWGQGTLVTVSA | |
| GC33 | HCVR | QVQLQQSGAELVRPGASVKLSCKASGYTFTDYEMHWVKQTP | 48 |
| GC33G34A | HCVR | VHGLKWIGALDPKTGDTAYSQKFKGKATLTADKSSSTAYME | |
| GC33G34D | HCVR | LRSLTSEDSAVYYCTRFYSYTYWGQGTLVTVSA | |
| GC33G34E | HCVR | | |
| GC33G34F | HCVR | | |
| GC33G34H | HCVR | | |
| GC33G34N | HCVR | | |
| GC33G34T | HCVR | | |
| GC33G34Q | HCVR | | |
| GC33G34I | HCVR | | |
| GC33G34K | HCVR | | |
| GC33G34L | HCVR | | |
| GC33G34S | HCVR | | |
| GC33G34W | HCVR | | |
| GC33G34Y | HCVR | | |
| GC33G34R | HCVR | | |
| GC33G34V | HCVR | | |
| GC33G34P | HCVR | | |
| GC179 | HCVR | EVQLVETGGGLVQPEGSLKLSCAASGFSFNINAMNWVRQAPG | 49 |
| | | KGLEWVARIRSESNNYATYYGDSVKDRFTISRDDSQNMLYLQ | |
| | | MNNLKTEDTAIYYCVREVTTSFAYWGQGTLVTVSA | |
| GC194 (1) | HCVR | EVQLVETGGGLVQPKGSLKLSCAASGFTFNASAMNWVRQAP | 50 |
| GC194 (2) | | GKGLEWVARIRSKSNNYAIYYADSVKDRFTISRDDSQSMLYL | |
| | | QMNNLKTEDTAMYYCVRDPGYYGNPWFAYWGQGTLVTVSA | |
| GV33va* | HCVR | QVQLVESGAEVKKPGASVKVSCKASGYTFTDYEMHWVRQAP | 51 |
| | | GQGLEWMGALDPKTGDTAYSQKFKGRVTITADESTSTAYME | |
| | | LSSLRSEDTAVYYCARFYSYTYWGQGTLVTVSS | |
| GC33vc* | HCVR | QVQLVESGAEVKKPGASVKVSCKASGYTFTDYEMHWVRQAP | 52 |
| | | GQGLEWMGALDPKTGDTAYSQKFKGRVTLTADESTSTAYME | |
| | | LSSLRSEDTAVYYCTRFYSYTYWGQGTLVTVSS | |
| GC33vf* | HCVR | QVQLVESGAEVKKPGASVKVSCKASGYTFTDYEMHWVRQAP | 53 |
| | | GQGLEWMGALDPKTGDTAYSQKFKGRVTLTADKSTSTAYME | |
| | | LSSLRSEDTAVYYCTRFYSYTYWGQGTLVTVSS | |
| GC33vh* | HCVR | QVQLVESGAEVKKPGASVKVSCKASGYTFTDYEMHWVRQAP | 54 |
| | | GQGLEWMGALDPKTGDTAYSQKFKGRVTLTADKSTSTAYME | |
| | | LSSLTSEDTAVYYCTRFYSYTYWGQGTLVTVSS | |
| GC33vi* | HCVR | QVQLVQSGAEVKKPGASVKVSCKASGYTFTDYEMHWVRQA | 55 |
| | | PGQGLEWMGALDPKTGDTAYSQKFKGRVTLTADESTSTAYM | |
| | | ELSSLRSEDTAVYYCTRFYSYTYWGQGTLVTVSS | |
| GC33vj* | HCVR | QVQLVQSGAEVKKPGASVKVSCKASGYTFTDYEMHWVRQA | 56 |
| | | PGQGLEWMGALDPKTGDTAYSQKFKGRVTLTADKSTSTAYM | |
| | | ELSSLRSEDTAVYYCTRFYSYTYWGQGTLVTVSS | |
| GC33vk* | HCVR | QVQLVQSGAEVKKPGASVKVSCKASGYTFTDYEMHWVRQA | 57 |
| | | PGQGLEWMGALDPKTGDTAYSQKFKGRVTLTADKSTSTAYM | |
| | | ELSSLTSEDTAVYYCTRFYSYTYWGQGTLVTVSS | |
| M13B3 | CDR-L1 | KSSQSLLDSDGKTYLN | 58 |
| | CDR-L2 | LVSKLDS | 59 |
| | CDR-L3 | WQGTHFPLT | 60 |
| M3B8 | CDR-L1 | KASQDINNYLS | 61 |
| | CDR-L2 | RANRLVD | 62 |
| | CDR-L3 | LQCDEFPPWT | 63 |
| M11F1 | CDR-L1 | RSSQSLVHSNGNTYLH | 64 |
| | CDR-L2 | KVSNRFS | 65 |
| | CDR-L3 | SQSTHVPWT | 66 |
| M6B1 | CDR-L1 | KASQDINKNII | 67 |
| | CDR-L2 | YTSTLQP | 68 |
| | CDR-L3 | LQYDNLPRT | 69 |
| M5B9 | CDR-L1 | RSSKSLLHSNGITYLY | 70 |
| | CDR-L2 | QMSNLAS | 71 |
| | CDR-L3 | AQNLELPYT | 72 |
| M10D2 | CDR-L1 | RASHSISNFLH | 73 |
| | CDR-L2 | YASQSIS | 74 |
| | CDR-L3 | QQSNIWSLT | 75 |
| L9G11 | CDR-L1 | RASESVEYYGTSLMQ | 76 |
| | CDR-L2 | GASNVES | 77 |
| | CDR-L3 | QQSRKVPYT | 78 |
| GC199 | CDR-L1 | KSSQSLLHSDGKTFLN | 79 |
| | CDR-L2 | LVSRLDS | 80 |
| | CDR-L3 | CQGTHFPRT | 81 |
| GC202 | CDR-L1 | RSSQSIVHSNGNTYLE | 82 |
| | CDR-L3 | FQGSHVPWT | 83 |
| GC33 | CDR-L3 | SQNTHVPPT | 84 |
| GC33G34A | CDR-L1 | RSSQSLVHSNANTYLH | 85 |
| GC33G34D | CDR-L1 | RSSQSLVHSNDNTYLH | 86 |
| GC33G34E | CDR-L1 | RSSQSLVHSNENTYLH | 87 |
| GC33G34F | CDR-L1 | RSSQSLVHSNFNTYLH | 88 |
| GC33G34H | CDR-L1 | RSSQSLVHSNHNTYLH | 89 |
| GC33G34N | CDR-L1 | RSSQSLVHSNNNTYLH | 90 |
| GC33G34T | CDR-L1 | RSSQSLVHSNTNTYLH | 91 |
| GC33G34Q | CDR-L1 | RSSQSLVHSNQNTYLH | 92 |
| GC33G34I | CDR-L1 | RSSQSLVHSNINTYLH | 93 |
| GC33G34K | CDR-L1 | RSSQSLVHSNKNTYLH | 94 |
| GC33G34L | CDR-L1 | RSSQSLVHSNLNTYLH | 95 |
| GC33G34S | CDR-L1 | RSSQSLVHSNSNTYLH | 96 |
| GC33G34W | CDR-L1 | RSSQSLVHSNWNTYLH | 97 |
| GC33G34Y | CDR-L1 | RSSQSLVHSNYNTYLH | 98 |
| GC33G34R | CDR-L1 | RSSQSLVHSNRNTYLH | 99 |
| GC33G34V | CDR-L1 | RSSQSLVHSNVNTYLH | 100 |
| GC33G34P | CDR-L1 | RSSQSLVHSNPNTYLH | 101 |
| GC179 | CDR-L1 | KSSKSLLHSNGNTYLN | 102 |
| | CDR-L2 | WMSNLAS | 103 |
| | CDR-L3 | MQHIEYPFT | 104 |
| GC194 (1) | CDR-L1 | RSSKSLLHSYDITYLY | 105 |
| | CDR-L3 | AQNLELPPT | 106 |
| GC194 (2) | CDR-L1 | SASSSVSYMY | 107 |
| | CDR-L2 | DTSNLAS | 108 |
| | CDR-L3 | QQWSSYPLT | 109 |
| MC311 | LCVR | MSPAQFLFLLVLWIRETNGDVVMTQTPLTLSVTIGQPASISCKS | 110 |
| | | SQSLLDSDGKTYLNWLLQRPGQSPKRLIYLVSKLDSGAPDRFT | |
| | | GSGSGTDFTLKISRVEAEDLGIYYCWQGTHFPLTFGAGTKLEL | |
| | | KRTVAAPSVFIFPPSDEQLKSGTASVVCLLNNFYPREAKVQWK | |
| | | VDNALQSGNSQESVTEQDSKDSTYSLSSTLTLSKADYEKHKV | |
| | | YACEVTHQGLSSPVTKSFNRGEC | |
| M13B3 | LCVR | DVVMTQSPLTLSITIGQPASISCKSSQSLLDSDGKTYLNWLLQR | 111 |
| | | PGQSPKRLIYLVSKLDSGVPDRFTGSGSGTDFSLKISR VEAEDL | |
| | | GIYYCWQGTHFPLTFGAGTKLELK | |
| M1E7 | LCVR | MSPVQFLFLLMLWIQETNGDVVMTQTPLSLSVTIGQPASISCK | 112 |
| | | SSQSLLYSNGKTYLNWLQQRPGQAPKHLMYQVSKLDPGIPDR | |
| | | FSGSGSETDFTLKISRVEAEDLGVYYCLQSTYYPLTFGAGTKL | |
| | | ELKRTVAAPSVFIFPPSDEQLKSGTASVVCLLNNFYPREAKVQ | |
| | | WKVDNALQSGNSQESVTEQDSKDSTYSLSSTLTLSKADYEKH | |
| | | KVYACEVTHQGLSSPVTKSFNRGEC | |
| M3B8 | LCVR | DIKMTQSPSSMYASLGERVTITCKASQDINNYLSWFQQKPGKS | 113 |
| | | PKTLIYRANRLVDGVPSRFSGSGSGQDYSLTISSLEYEDMGINY | |
| | | CLQCDEFPPWTFGGGTKLEIK | |
| M11F1 | LCVR | DVVMTQTPLSLPVSLGDQASISCRSSQSLVHSNGNTYLHWYL | 114 |
| | | QKPGQSPKLLIYKVSNRFSGVPDRFSGSGSGTDFTLKISRVEAE | |
| | | DLGVYFCSQSTHVPWTFGGGTKLEIK | |
| M19B11 | LCVR | MRPSIQFLGLLLFWLHGVQCDIQMTQSPSSLSASLGGKVTITC | 115 |
| | | KASQDINKNIVWYQHKPGKGPRLLIWYTSTLQPGIPSRFSGSG | |
| | | SGRDYSFSISNLEPEDIATYYCLQYDNLPRTFGGGTKLEIKRTV | |
| | | AAPSVFIFPPSDEQLKSGTASVVCLLNNFYPREAKVQWKVDN | |
| | | ALQSGNSQESVTEQDSKDSTYSLSSTLTLSKADYEKHKVYAC | |
| | | EVTHQGLSSPVTKSFNRGEC | |
| M6B1 | LCVR | DIQMTQSPSSLSASLGGKVTITCKASQDINKNIIWYQHKPGKGP | 116 |
| | | RLLIWYTSTLQPGIPSRFSGSGSGRDYSFSISNLEPEDIATYYCL | |
| | | QYDNLPRTFGGGTKLEIK | |
| M18D4 | LCVR | MRFSAQLLGLLVLWIPGSTADIVMTQAAFSNPVTLGTSTSISCR | 117 |
| | | SSKSLLHSNGITYLYWYLQKPGQSPQLLIYQMSNLASGVPDRF | |
| | | SSSGSGTDFTLRISRVEAEDVGVYYCAQNLELPYTFGSGTKLEI | |
| | | KRTVAAPSVFIFPPSDEQLKSGTASVVCLLNNFYPREAKVQWK | |
| | | VDNALQSGNSQESVTEQDSKDSTYSLSSTLTLSKADYEKHKV | |
| | | YACEVTHQGLSSPVTKSFNRGEC | |
| M5B9 | LCVR | DIVMTQAAFSNPVTLGTSASISCRSSKSLLHSNGITYLYWFLQK | 118 |
| | | PGQSPQLLIYQMSNLASGVPDRESSSGSGTDFTLRISRVEAEDV | |
| | | GVYYCAQNLELPYTFGSGTKLEIK | |
| M10D2 | LCVR | DIVLTQSPATLSVTPGDRVSLSCRASHSISNFLHWYPQKSHESP | 119 |
| | | RLLIKYASQSISGIPSRFSGNGSGTDFTLSINSVETEDFGMYFCQ | |
| | | QSNIWSLTFGAGTKLELK | |
| L9G11 | LCVR | DIVLTQSPTSLAVSLGQSVTISCRASESVEYYGTSLMQWYQQK | 120 |
| | | PGQPPKLLIYGASNVESGVPARFSGSGSGTDFSLNIHPVEEDDI | |
| | | AMYFCQQSRKVPYTFGSGTKLEIK | |
| GC199 | LCVR | DVVMTQTPLTLSVTLGQPASISCKSSQSLLHSDGKTFLNWLLQ | 121 |
| | | RPGQSPKRLIYLVSRLDSGVPDRFTGSGSGTDFTLKISRVEAED | |
| | | LGVYYCCQGTHFPRTFGGGTRLEIK | |
| GC202 | LCVR | DVLMTQTPLSLPVSLGDQASISCRSSQSIVHSNGNTYLEWYLQ | 122 |
| | | KPGQSPKLLIYKVSNRFSGVPDRFSGSGSGTDFTLKISR VEAED | |
| | | LGVYYCFQGSHVPWTFGGGTKLEIK | |
| GC33 | LCVR | DVVMTQTPLSLPVSLGDQASISCRSSQSLVHSNGNTYLHWYL | 123 |
| | | QKPGQSPKLLIYKVSNRFSGVPDRFSGSGSGTDFTLKISRVEAE | |
| | | DLGVYFCSQNTHVPPTFGSGTKLEIK | |
| GC33G34A | LCVR | DVVMTQSPLSLPVTPGEPASISCRSSQSLVHSNANTYLHWYLQ | 124 |
| | | KPGQSPQLLIYKVSNRFSGVPDRFSGSGSGTDFTLKISRVEAED | |
| | | VGVYYCSQNTHVPPTFGQGTKLEIK | |
| GC33G34D | LCVR | DVVMTQSPLSLPVTPGEPASISCRSSQSLVHSNDNTYLHWYLQ | 125 |
| | | KPGQSPQLLIYKVSNRFSGVPDRFSGSGSGTDFTLKISRVEAED | |
| | | VGVYYCSQNTHVPPTFGQGTKLEIK. | |
| GC33G34E | LCVR | DVVMTQSPLSLPVTPGEPASISCRSSQSLVHSNENTYLHWYLQ | 126 |
| | | KPGQSPQLLIYKVSNRFSGVPDRFSGSGSGTDFTLKISR VEAED | |
| | | VGVYYCSQNTHVPPTFGQGTKLEIK | |
| GC33G34F | LCVR | DVVMTQSPLSLPVTPGEPASISCRSSQSLVHSNFNTYLHWYLQ | 127 |
| | | KPGQSPQLLIYKVSNRFSGVPDRFSGSGSGTDFTLKISRVEAED | |
| | | VGVYYCSQNTHVPPTFGQGTKLEIK | |
| GC33G34H | LCVR | DVVMTQSPLSLPVTPGEPASISCRSSQSLVHSNHNTYLHWYLQ | 128 |
| | | KPGQSPQLLIYKVSNRFSGVPDRFSGSGSGTDFTLKISRVEAED | |
| | | VGVYYCSQNTHVPPTFGQGTKLEIK | |
| GC33G34N | LCVR | DVVMTQSPLSLPVTPGEPASISCRSSQSLVHSNNNTYLHWYLQ | 129 |
| | | KPGQSPQLLIYKVSNRFSGVPDRFSGSGSGTDFTLKISRVEAED | |
| | | VGVYYCSQNTHVPPTFGQGTKLEIK | |
| GC33G34T | LCVR | DVVMTQSPLSLPVTPGEPASISCRSSQSLVHSNTNTYLHWYLQ | 130 |
| | | KPGQSPQLLIYKVSNRFSGVPDRFSGSGSGTDFTLKISRVEAED | |
| | | VGVYYCSQNTHVPPTFGQGTKLEIK | |
| GC33G34Q | LCVR | DVVMTQSPLSLPVTPGEPASISCRSSQSLVHSNQNTYLHWYLQ | 131 |
| | | KPGQSPQLLIYKVSNRFSGVPDRFSGSGSGTDFTLKISRVEAED | |
| | | VGVYYCSQNTHVPPTFGQGTKLEIK | |
| GC33G34I | LCVR | DVVMTQSPLSLPVTPGEPASISCRSSQSLVHSNINTYLHWYLQ | 132 |
| | | KPGQSPQLLIYKVSNRFSGVPDRFSGSGSGTDFTLKISRVEAED | |
| | | VGVYYCSQNTHVPPTFGQGTKLEIK | |
| GC33G34K | LCVR | DVVMTQSPLSLPVTPGEPASISCRSSQSLVHSNKNTYLHWYLQ | 133 |
| | | KPGQSPQLLIYKVSNRFSGVPDRFSGSGSGTDFTLKISRVEAED | |
| | | VGVYYCSQNTHVPPTFGQGTKLEIK | |
| GC33G34L | LCVR | DVVMTQSPLSLPVTPGEPASISCRSSQSLVHSNLNTYLHWYLQ | 134 |
| | | KPGQSPQLLIYKVSNRFSGVPDRFSGSGSGTDFTLKISR VEAED | |
| | | VGVYYCSQNTHVPPTFGQGTKLEIK | |
| GC33G34S | LCVR | DVVMTQSPLSLPVTPGEPASISCRSSQSLVHSNSNTYLHWYLQ | 135 |
| | | KPGQSPQLLIYKVSNRFSGVPDRFSGSGSGTDFTLKISRVEAED | |
| | | VGVYYCSQNTHVPPTFGQGTKLEIK | |
| GC33G34W | LCVR | DVVMTQSPLSLPVTPGEPASISCRSSQSLVHSNWNTYLHWYL | 136 |
| | | QKPGQSPQLLIYKVSNRFSGVPDRFSGSGSGTDFTLKISRVEAE | |
| | | DVGVYYCSQNTHVPPTFGQGTKLEIK | |
| GC33G34Y | LCVR | DVVMTQSPLSLPVTPGEPASISCRSSQSLVHSNYNTYLHWYLQ | 137 |
| | | KPGQSPQLLIYKVSNRFSGVPDRFSGSGSGTDFTLKISRVEAED | |
| | | VGVYYCSQNTHVPPTFGQGTKLEIK | |
| GC33G34R | LCVR | DVVMTQSPLSLPVTPGEPASISCRSSQSLVHSNRNTYLHWYLQ | 138 |
| | | KPGQSPQLLIYKVSNRFSGVPDRFSGSGSGTDFTLKISRVEAED | |
| | | VGVYYCSQNTHVPPTFGQGTKLEIK | |
| GC33G34V | LCVR | DVVMTQSPLSLPVTPGEPASISCRSSQSLVHSNVNTYLHWYLQ | 139 |
| | | KPGQSPQLLIYKVSNRFSGVPDRFSGSGSGTDFTLKISRVEAED | |
| | | VGVYYCSQNTHVPPTFGQGTKLEIK | |
| GC33G34P | LCVR | DVVMTQSPLSLPVTPGEPASISCRSSQSLVHSNPNTYLHWYLQ | 140 |
| | | KPGQSPQLLIYKVSNRFSGVPDRFSGSGSGTDFTLKISR VEAED | |
| | | VGVYYCSQNTHVPPTFGQGTKLEIK | |
| GC179 | LCVR | DIVMTQSAPSVPVTPGESVSISCKSSKSLLHSNGNTYLNWFLQ | 141 |
| | | RPGQSPQLLIYWMSNLASGVPDRFSGSGSGTAFTLRISRVEAE | |
| | | DVGVYYCMQHIEYPFTFGTGTKLEIK | |
| GC194 (1) | LCVR | DIVMTQAAFSNPVTLGTSASISCRSSKSLLHSYDITYLYWYLQ | 142 |
| | | KPGQSPQLLIYQMSNLASGVPDRFSSSGSGTDFTLRISRVEAED | |
| | | VGVYYCAQNLELPPTFGGGTKLEIK | |
| GC194 (2) | LCVR | QIVLTQSPAIMSAFPGEKVTMTCSASSSVSYMYWYQQKSGSSP | 143 |
| | | RLLIYDTSNLASGVPVRFSGSGSGTSYSLTISRMEAEDAATYY | |
| | | CQQWSSYPLTFGGGTELELK | |
| GV33va* | LCVR | DVVMTQSPLSLPVTPGEPASISCRSSQSLVHSNGNTYLHWYLQ | 144 |
| GC33vc* | LCVR | KPGQSPQLLIYKVSNRFSGVPDRFSGSGSGTDFTLKISRVEAED | |
| GC33vf* | LCVR | VGVYYCSQNTHVPPTFGQGTKLEIK | |
| GC33vh* | LCVR | | |
| GC33vi* | LCVR | | |
| GC33vj* | LCVR | | |
| GC33vk* | LCVR | | |
| 4A6 | CDR-H1 | SYWIA | 145 |
| | CDR-H2 | IIFPGDSDTRYSPSFQG | 146 |
| | CDR-H3 | TREGYFDY | 147 |
| 11-E7 | CDR-H1 | NYWIA | 148 |
| | CDR-H2 | IIYPGDSDTRYSPSFQG | 149 |
| 16D10 | CDR-H1 | NYWIG | 150 |
| | CDR-H3 | TREGFFDY | 151 |
| 4A6 | HCVR | EVQLVQSGAEVKKPGESLKISCKGSGYSFTSYWIAWVRQMPG | 152 |
| | | KGLEWMGIIFPGDSDTRYSPSFQGQVTISADRSIRTAYLQWSSL | |
| | | KASDTALYYCARTREGYFDYWGQGTLVTVSS | |
| 11-E7 | HCVR | EVQLVQSGAEVKKPGESLKISCKGSGYSFTNYWIAWVRQMPG | 153 |
| | | KGLEWMGIIYPGDSDTRYSPSFQGQVTISADKSIRTAYLQWSS | |
| | | LKASDTAMYYCARTREGYFDYWGQGTLVTVSS | |
| 16D10 | HCVR | EVQLVQSGADVTKPGESLKISCKVSGYRFTNYWIGWMRQMS | 154 |
| | | GKGLEWMGIIYPGDSDTRYSPSFQGHVTISADKSINTAYLRWS | |
| | | SLKASDTAIYYCARTREGFFDYWGQGTPVTVSS | |
| 4A6 | CDR-L1 | RAVQSVSSSYLA | 155 |
| 4A6 | CDR-L2 | GASSRAT | 156 |
| 11-E7 | | | |
| 16D10 | | | |
| 4A6 | CDR-L3 | QQYGSSPT | 157 |
| 11-E7 | | | |
| 16D10 | | | |
| 11-E7 | CDR-L1 | RASQSVSSSYLA | 158 |
| 16D10 | | | |
| 4A6 | LCVR | EIVLTQSPGTLSLSPGERATLSCRAVQSVSSSYLAWYQQKPGQ | 159 |
| | | APRLLIYGASSRATGIPDRFSGSGSGTDFTLTISRLEPEDFAVYY | |
| | | CQQYGSSPTFGGGTKVEIK | |
| 11-E7 | LCVR | EIVLTQSPGTLSLSPGERATLSCRASQSVSSSYLAWYQQKPGQ | 160 |
| | | APRLLIYGASSRATGIPDRFSGSGSGTDFTLTISRLEPEDFAVYY | |
| | | CQQYGSSPTFGGGTKVEIK | |
| 16D10 | LCVR | EILLTQSPGTLSLSPGERATLSCRASQSVSSSYLAWYQQKPGQ | 161 |
| | | APRLLIYGASSRATGIPDRFSGSGSGTDFTLTISRLEPEDFAVYY | |
| | | CQQYGSSPTFGQGTKVEIK | |

*Humanized GC33 antibodies that maintain the murine GC33 CDR sequences

In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of an amino acid sequence selected from SEQ ID NO: 35-57 and 152-154.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 35. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 35.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 36. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 36.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 37. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 37.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 38. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 38.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 39. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 39.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 40. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 40.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 41. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 41.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 42. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 42.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 43. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 43.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 44. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 44.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 45. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 45.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 46. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 46.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 47. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 47.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 48.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 49. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 49.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 50. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 50.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 51. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 51.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 52. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 52.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 53. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 53.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 54. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 54.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 55. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 55.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 56. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 56.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 57. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 57.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 152. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 152.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 153. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 153.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 154. In some embodiments, the VH region comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 154.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from SEQ ID NOs: 36, 38, 39, 41, 43, 44, 45, 46, 47, 48, 49, 50, 152, 153, and 154.

In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of an amino acid sequence selected from the group SEQ ID NO: 110-144 and 159-161.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 110. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 110.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 111. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 111.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 112. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 112.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 113. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 113.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 114. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 114.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 115. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 115.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 116. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 116.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 117. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 117.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 118. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 118.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 119. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 119.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 120. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 120.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 121. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 121.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 122. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 122.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 123. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 123.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 124. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 124.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 125. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 125.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 26. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 126.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 127. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 127.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 128. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 128.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 129. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 129.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 130. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 130.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 131. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 131.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 132. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 132.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 133. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 133.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 134. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 134.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 135. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 135.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 136. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 136.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 137. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 137.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 138. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 138.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 139. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 139.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 140. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 140.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 141. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 141.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 142. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 142.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 143. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 143.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 159. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 159.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 160. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 160.

In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 161. In some embodiments, the VL region comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 161. In some embodiments, the VL region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from SEQ ID NOs: 111, 113, 114, 116, 118-143, and 159-161.

In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of an amino acid sequence selected from SEQ ID NO: 35-57 and 152-154; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of an amino acid sequence selected from SEQ ID NO: 110-144 and 159-161.

In some embodiments, the VH comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from SEQ ID NO: 35-57 and 152-154; and the VL comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from SEQ ID NO: 110-144 and 159-161.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 35 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 110. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 35; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 110. In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 36 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 111. In some embodiments, the VH comprises a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 36; and the VL comprises a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 111. In some embodiments, the VH comprises a heavy chain complementarity determining region 1 (CDR-H1) having the amino acid sequence of SEQ ID NO:1, a heavy chain complementarity determining region 2 (CDR-H2) having the amino acid sequence of SEQ ID NO: 2, and a heavy chain complementarity determining region 3 (CDR-H3) having the amino acid sequence of SEQ ID NO: 3. In some embodiments, the VL comprises a light chain complementarity determining region 1 (CDR-L1) having the amino acid sequence of SEQ ID NO: 58, a light chain complementarity determining region 2 (CDR-L2) having the amino acid sequence of SEQ ID NO:59, and a light chain complementarity determining region 3 (CDR-L3) having the amino acid sequence of SEQ ID NO: 60.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 37 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 112. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 37; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 112.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 38 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 113. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 38; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 113. In some embodiments, the VH includes a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 5, and a heavy chain complementarity determining region 3 (CDR-H3) having the amino acid sequence of SEQ ID NO: 6. In some embodiments, the VL includes a light chain complementarity determining region 1 (CDR-L1) having the amino acid sequence of SEQ ID NO: 61, a light chain complementarity determining region 2 (CDR-L2) having the amino acid sequence of SEQ ID NO: 62, and a light chain complementarity determining region 3 (CDR-L3) having the amino acid sequence of SEQ ID NO: 63.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 39 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 114. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 39; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 114. In some embodiments, the VH includes a heavy chain complementarity determining region 1 (CDR-H1) having the amino acid sequence of SEQ ID NO: 7, a heavy chain complementarity determining region 2 (CDR-H2) having the amino acid sequence of SEQ ID NO: 8, and a heavy chain complementarity determining region 3 (CDR-H3) having the amino acid sequence of SEQ ID NO: 9. In some embodiments, the VL includes a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 66.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 40 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 115. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 40; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 115.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 41 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 116. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 41; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 116. In some embodiments, the VH includes a heavy chain complementarity determining region 1 (CDR-H1) having the amino acid sequence of SEQ ID NO: 10, a heavy chain complementarity determining region 2 (CDR-H2) having the amino acid sequence of SEQ ID NO: 11, and a heavy chain complementarity determining region 3 (CDR-H3) having the amino acid sequence of SEQ ID NO: 12. In some embodiments, the VL includes a light chain complementarity determining region 1 (CDR-L1) having the amino acid sequence of SEQ ID NO: 67, a light chain complementarity determining region 2 (CDR-L2) having the amino acid sequence of SEQ ID NO: 68, and a light chain complementarity determining region 3 (CDR-L3) having the amino acid sequence of SEQ ID NO: 69.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 42 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 116. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 42; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 116.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 43 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 118. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 43; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 118. In some embodiments, the VH includes a heavy chain complementarity determining region 1 (CDR-H1) having the amino acid sequence of SEQ ID NO: 13, a heavy chain complementarity determining region 2 (CDR-H2) having the amino acid sequence of SEQ ID NO: 14, and a heavy chain complementarity determining region 3 (CDR-H3) having the amino acid sequence of SEQ ID NO: 15. In some embodiments, the VL includes a light chain complementarity determining region 1 (CDR-L1) having the amino acid sequence of SEQ ID NO: 70, a light chain complementarity determining region 2 (CDR-L2) having the amino acid sequence of SEQ ID NO: 71, and a light chain complementarity determining region 3 (CDR-L3) having the amino acid sequence of SEQ ID NO: 72.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 44 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 119. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 44; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 119. In some embodiments, the VH includes a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 17, and a heavy chain complementarity determining region 3 (CDR-H3) having the amino acid sequence of SEQ ID NO: 18. In some embodiments, the VL includes a light chain complementarity determining region 1 (CDR-L1) having the amino acid sequence of SEQ ID NO: 73, a light chain complementarity determining region 2 (CDR-L2) having the amino acid sequence of SEQ ID NO: 74, and a light chain complementarity determining region 3 (CDR-L3) having the amino acid sequence of SEQ ID NO: 75.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 45 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 120. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 45; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 120. In some embodiments, the VH includes a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 19, and a heavy chain complementarity determining region 3 (CDR-H3) having the amino acid sequence of SEQ ID NO: 20. In some embodiments, the VL includes a light chain complementarity determining region 1 (CDR-L1) having the amino acid sequence of SEQ ID NO: 76, a light chain complementarity determining region 2 (CDR-L2) having the amino acid sequence of SEQ ID NO: 77, and a light chain complementarity determining region 3 (CDR-L3) having the amino acid sequence of SEQ ID NO: 78.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 46 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 121. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 46; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 121. In some embodiments, the VH includes a heavy chain complementarity determining region 1 (CDR-H1) having the amino acid sequence of SEQ ID NO: 21, a heavy chain complementarity determining region 2 (CDR-H2) having the amino acid sequence of SEQ ID NO: 22, and a heavy chain complementarity determining region 3 (CDR-H3) having the amino acid sequence of SEQ ID NO: 23. In some embodiments, the VL includes a light chain complementarity determining region 1 (CDR-L1) having the amino acid sequence of SEQ ID NO: 79, a light chain complementarity determining region 2 (CDR-L2) having the amino acid sequence of SEQ ID NO: 80, and a light chain complementarity determining region 3 (CDR-L3) having the amino acid sequence of SEQ ID NO: 81.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 47 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 122. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 47; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 122. In some embodiments, the VH includes a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 24, and a heavy chain complementarity determining region 3 (CDR-H3) having the amino acid sequence of SEQ ID NO: 25. In some embodiments, the VL includes a light chain complementarity determining region 1 (CDR-L1) having the amino acid sequence of SEQ ID NO: 82, a light chain complementarity determining region 2 (CDR-L2) having the amino acid sequence of SEQ ID NO: 65, and a light chain complementarity determining region 3 (CDR-L3) having the amino acid sequence of SEQ ID NO: 83.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 123. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 123. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 124. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 124. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 85, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 125. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 125. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 86, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 126. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 126. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 87, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 127. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 127. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 88, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 128. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 128. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 89, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 129. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 129. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 90, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 130. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 130. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 91, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 131. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 131. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 92, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 132. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 132. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 93, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 133. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 133. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 94, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 134. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 134. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 95, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 135. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 135. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 96, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 136. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 136. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 97, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 137. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 137. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 98, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 138. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 138. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 99, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 139. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 139. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 100, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 48 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 140. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 48; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 140. In some embodiments, the VH includes a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, and a heavy chain complementarity determining region 3 (CDR-H3) having the amino acid sequence of SEQ ID NO: 28. In some embodiments, the VL includes a light chain complementarity determining region 1 (CDR-L1) having the amino acid sequence of SEQ ID NO: 101, a light chain complementarity determining region 2 (CDR-L2) having the amino acid sequence of SEQ ID NO: 65, and a light chain complementarity determining region 3 (CDR-L3) having the amino acid sequence of SEQ ID NO: 84.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 49 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 141. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 49; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 141. In some embodiments, the VH includes a heavy chain complementarity determining region 1 (CDR-H1) having the amino acid sequence of SEQ ID NO: 29, a heavy chain complementarity determining region 2 (CDR-H2) having the amino acid sequence of SEQ ID NO: 30, and a heavy chain complementarity determining region 3 (CDR-H3) having the amino acid sequence of SEQ ID NO: 31. In some embodiments, the VL includes a light chain complementarity determining region 1 (CDR-L1) having the amino acid sequence of SEQ ID NO: 102, a light chain complementarity determining region 2 (CDR-L2) having the amino acid sequence of SEQ ID NO: 103, and a light chain complementarity determining region 3 (CDR-L3) having the amino acid sequence of SEQ ID NO: 104.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 50 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 142. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 50; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 142. In some embodiments, the VH includes a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, and a heavy chain complementarity determining region 3 (CDR-H3) having the amino acid sequence of SEQ ID NO: 34. In some embodiments, the VL includes a light chain complementarity determining region 1 (CDR-L1) having the amino acid sequence of SEQ ID NO: 105, a light chain complementarity determining region 2 (CDR-L2) having the amino acid sequence of SEQ ID NO: 71, and a light chain complementarity determining region 3 (CDR-L3) having the amino acid sequence of SEQ ID NO: 106.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 50 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 143. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 50; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 143. In some embodiments, the VH includes a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, and a heavy chain complementarity determining region 3 (CDR-H3) having the amino acid sequence of SEQ ID NO: 34. In some embodiments, the VL includes a light chain complementarity determining region 1 (CDR-L1) having the amino acid sequence of SEQ ID NO: 107, a light chain complementarity determining region 2 (CDR-L2) having the amino acid sequence of SEQ ID NO: 108, and a light chain complementarity determining region 3 (CDR-L3) having the amino acid sequence of SEQ ID NO: 109.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 51 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 51; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 52 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 52; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 53 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 53; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 54 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 54; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 55 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 55; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 56 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 56; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 57 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 144. In some embodiments, the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of the amino acid sequence of SEQ ID NO: 57; and the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of the amino acid sequence of SEQ ID NO: 144.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 152 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 159. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 152; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 159. In some embodiments, the VH includes a heavy chain complementarity determining region 1 (CDR-H1) having the amino acid sequence of SEQ ID NO: 145, a heavy chain complementarity determining region 2 (CDR-H2) having the amino acid sequence of SEQ ID NO: 146, and a heavy chain complementarity determining region 3 (CDR-H3) having the amino acid sequence of SEQ ID NO: 147. In some embodiments, the VL includes a light chain complementarity determining region 1 (CDR-L1) having the amino acid sequence of SEQ ID NO: 155, a light chain complementarity determining region 2 (CDR-L2) having the amino acid sequence of SEQ ID NO: 156, and a light chain complementarity determining region 3 (CDR-L3) having the amino acid sequence of SEQ ID NO: 157.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 153 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 160. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 153; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 160. In some embodiments, the VH includes a heavy chain complementarity determining region 1 (CDR-H1) having the amino acid sequence of SEQ ID NO: 148, a heavy chain complementarity determining region 2 (CDR-H2) having the amino acid sequence of SEQ ID NO: 149, and a heavy chain complementarity determining region 3 (CDR-H3) having the amino acid sequence of SEQ ID NO: 147. In some embodiments, the VL includes a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157.

In some embodiments, the VH region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 154 and the VL region comprises an amino acid sequence with at least 90%, at least 91%, at least 92%, at least 93%, at least 94%, at least 95%, at least 96%, at least 97%, at least 98%, at least 99%, or 100% identity to the amino acid sequence of SEQ ID NO: 161. In some embodiments, the VH includes a heavy chain complementarity determining region 1 (CDR-H1), a heavy chain complementarity determining region 2 (CDR-H2), and a heavy chain complementarity determining region 3 (CDR-H3) having the amino acid sequences of CDR-H1, CDR-H2, and CDR-H3 contained within the VH region amino acid sequence of SEQ ID NO: 154; and the VL includes a light chain complementarity determining region 1 (CDR-L1), a light chain complementarity determining region 2 (CDR-L2), and a light chain complementarity determining region 3 (CDR-L3) having the amino acid sequences of CDR-L1, CDR-L2, and CDR-L3 contained within the VL region amino acid sequence of SEQ ID NO: 161. In some embodiments, the VH includes a heavy chain complementarity determining region 1 (CDR-H1) having the amino acid sequence of SEQ ID NO: 150, a heavy chain complementarity determining region 2 (CDR-H2) having the amino acid sequence of SEQ ID NO: 149, and a heavy chain complementarity determining region 3 (CDR-H3) having the amino acid sequence of SEQ ID NO: 151. In some embodiments, the VL includes a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157.

In some embodiments, the VH and the VL are separated by a peptide linker. Exemplary peptide linkers are provided in Table 2. In some embodiments, the peptide linker includes an amino acid sequence selected from SEQ ID NOs: 162-180.

TABLE 2
Peptide Linker Amino Acid Sequences

| | SEQ ID | |
|---|---|---|
| Linker Sequence | NO: | Name |
| GGS | 162 | (G₂S)₁ linker |
| GGSGGS | 163 | (G₂S)₂ linker |
| GGSGGSGGS | 164 | (G₂S)₃ linker |
| GGSGGSGGSGGS | 165 | (G₂S)₄ linker |
| GGSGGSGGSGGSGGS | 166 | (G₂S)₅ linker |
| GGGS | 167 | (G₃S)₁ linker |
| GGGSGGGS | 168 | (G₃S)₂ linker |
| GGGSGGGSGGGS | 169 | (G₃S)₃ linker |
| GGGSGGGSGGGSGGGS | 170 | (G₃S)₄ linker |
| GGGSGGGSGGGSGGGSGGGS | 171 | (G₃S)₅ linker |
| GGGGS | 172 | (G₄S)₁ linker |
| GGGGSGGGGS | 173 | (G₄S)₂ linker |
| GGGGSGGGGSGGGGS | 174 | (G₄S)₃ linker |
| GGGGSGGGGSGGGGSGGGGS | 175 | (G₄S)₄ linker |
| GGGGSGGGGSGGGGSGGGGS | 176 | (G₄S)₅ linker |
| GGGGS | | |
| GSTSGSGKPGSGEGSTKG | 177 | linker |
| EAAAKEAAAKEAAAKEAAAK | 178 | linker |
| GGGGSGGGGSGGGGSVDGF | 179 | linker |
| ASGGGGSAS | 180 | linker |

GPC3 Chimeric Antigen Receptors

In some embodiments, the GPC3 CARs of the present disclosure comprise a GPC3 binding domain of the present disclosure and one or more intracellular signaling domains, where the one or more intracellular signaling domains may be selected from: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain. In some embodiments, the CAR comprises a CD3zeta-chain intracellular signaling domain and one or more additional intracellular signaling domains (e.g., co-stimulatory domains) selected from a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2 DS1 intracellular signaling domain, a KIR3 DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

In some embodiments, the CAR further comprises a transmembrane domain, and the transmembrane domain is selected from: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2 DS1 transmembrane domain, a KIR3 DS1 transmembrane domain, an NKp44 transmembrane domain, an NKp46 transmembrane domain, an FceR1g transmembrane domain, and an NKG2D transmembrane domain.

In some embodiments, the CAR further comprises a spacer region (e.g., hinge domain) between the antigen-binding domain and the transmembrane domain. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domains may include, without limitation an IgG domain (such as an IgG1 hinge, an IgG2 hinge, an IgG3 hinge, or an IgG4 hinge), an IgD hinge domain, a CD8a hinge domain, and a CD28 hinge domain. In some embodiments, the spacer or hinge domain is an IgG domain, an IgD domain, a CD8a hinge domain, or a CD28 hinge domain.

Exemplary spacer or hinge domain protein sequences are shown in Table 3.

TABLE 3
Spacer or Hinge Amino Acid Sequences

| | SEQ ID | |
|---|---|---|
| Spacer/Hinge Domains | NO: | Name |
| AAAIEVMYPPPYLDNEKSNGT | 181 | CD28 hinge |
| IIHVKGKHLCPSPLFPGPSKP | | |
| ESKYGPPCPSCP | 182 | IgG4 minimal hinge |
| ESKYGPPAPSAP | 183 | IgG4 minimal hinge, |
| | | no disulfides |
| ESKYGPPCPPCP | 184 | IgG4 S228P minimal |
| | | hinge, enhanced |
| | | disulfide formation |
| EPKSCDKTHTCP | 185 | IgG1 minimal hinge |
| AAAFVPVFLPAKPTTTPAPRP | 186 | Extended CD8a hinge |
| PTPAPTIASQPLSLRPEACRP | | |
| AAGGAVHTRGLDFACDIYIWA | | |
| PLAGTCGVLLLSLVITLYCNH | | |
| RN | | |
| TTTPAPRPPTPAPTIALQPLS | 187 | CD8a hinge |
| LRPEACRPAAGGAVHTRGLDF | | |
| ACD | | |
| ACPTGLYTHSGECCKACNLGE | 188 | LNGFR hinge |
| GVAQPCGANQTVCEPCLDSVT | | |
| FSDVVSATEPCKPCTECVGLQ | | |
| SMSAPCVEADDAVCRCAYGYY | | |
| QDETTGRCEACRVCEAGSGLV | | |
| FSCQDKQNTVCEECPDGTYSD | | |
| EADAEC | | |
| ACPTGLYTHSGECCKACNLGE | 189 | Truncated LNGFR |
| GVAQPCGANQTVC | | hinge (TNFR-Cys1) |
| AVGQDTQEVIVVPHSLPFKV | 190 | PDGFR-beta |
| | | extracellular |
| | | linker |

In some embodiments, the CAR includes a spacer region having an amino acid sequence selected from SEQ ID NOs: 181-190.

Suitable transmembrane domains, spacer or hinge domains, and intracellular domains for use in a CAR are generally described in Stoiber et al, Cells 2019, 8(5), 472; Guedan et al, Mol Therapy: Met & Clinic Dev, 2019 12:145-156; and Sadelain et al, Cancer Discov; 2013, 3(4); 388-98, each of which are hereby incorporated by reference in their entirety.

In some embodiments, the CAR further comprises a secretion signal peptide. Any suitable secretion signal peptide of the present disclosure may be used.

Nucleic Acid Molecules Encoding GPC3 CARs

In accordance with the above embodiments, the present disclosure provides nucleic acid molecules encoding any of the GPC3 CARs described herein. In some embodiments, the present disclosure provides an engineered nucleic acid comprising an expression cassette that includes a promoter operably linked to an exogenous polynucleotide sequence encoding a GPC3 CAR. As used herein, “promoter” generally refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid of the present disclosure may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 4, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Non-limiting examples of components of inducible promoters include those shown in Table 5.

TABLE 4
Exemplary Inducible Promoters

| | Promoter and | Transcription | Inducer | Response to inducer | |
|---|---|---|---|---|---|
| System | operator | factor (TF) | molecule | TF | T |
| Transcriptional activator-responsive promoters | | | | | |
| AIR | PAIR (OalcA- | AlcR | Acetaldehyde | n.d. | A |
| | PhCMVmin) | | | | |
| ART | PART (OARG- | ArgR-VP16 | 1-Arginine | B | A |
| | PhCMVmin) | | | | |
| BIT | PBIT3 (OBirA3- | BIT (BirA-VP16) | Biotin | B | A |
| | PhCMVmin) | | | | |
| Cumate - activator | PCR5 (OCuO6- | cTA (CymR- | Cumate | D | DA |
| | PhCMVmin) | VP16) | | | |
| Cumate - reverse | PCR5 (OCuO6- | rcTA (rCymR- | Cumate | B | A |
| activator | PhCMVmin) | VP16) | | | |
| E-OFF | PETR (OETR- | ET (E-VP16) | Erythromycin | D | DA |
| | PhCMVmin) | | | | |
| NICE-OFF | PNIC (ONIC- | NT (HdnoR-VP16) | 6-Hydroxy- | D | DA |
| | PhCMVmin) | | nicotine | | |
| PEACE | PTtgR1 (OTtgR- | TtgA1 (TtgR- | Phloretin | D | DA |
| | PhCMVmin) | VP16) | | | |
| PIP-OFF | PPIR (OPIR- | PIT (PIP-VP16) | Pristinamycin | D | DA |
| | Phsp70min) | | I | | |
| QuoRex | PSCA (OscbR- | SCA (ScbR-VP16) | SCB1 | D | DA |
| | PhCMVmin)PSPA | | | | |
| | (OpapRI-PhCMVmin) | | | | |
| Redox | PROP (OROP- | REDOX (REX- | NADH | D | DA |
| | PhCMVmin) | VP16) | | | |
| TET-OFF | PhCMV*-1 (OtetO7- | tTA (TetR-VP16) | Tetracycline | D | DA |
| | PhCMVmin) | | | | |
| TET-ON | PhCMV*-1 (OtetO7- | rtTA (rTetR-VP16) | Doxycycline | B | A |
| | PhCMVmin) | | | | |
| TIGR | PCTA (OrheO- | CTA (RheA-VP16) | Heat | D | DA |
| | PhCMVmin) | | | | |
| TraR | O7x(tra box)- | p65-TraR | 3-Oxo-C8- | B | A |
| | PhCMVmin | | HSL | | |
| VAC-OFF | P1VanO2 (OVanO2- | VanA1 (VanR- | Vanillic acid | D | DA |
| | PhCMVmin) | VP16) | | | |
| Transcriptional repressor-responsive promoters | | | | | |
| Cumate - repressor | PCuO (PCMV5- | CymR | Cumate | D | DR |
| | OCuO) | | | | |
| E-ON | PETRON8 (PSV40- | E-KRAB | Erythromycin | D | DR |
| | OETR8) | | | | |
| NICE-ON | PNIC (PSV40- | NS (HdnoR- | 6-Hydroxy- | D | DR |
| | ONIC8) | KRAB) | nicotine | | |
| PIP-ON | PPIRON (PSV40- | PIT3 (PIP-KRAB) | Pristinamycin | D | DR |
| | OPIR3) | | I | | |
| Q-ON | PSCAON8 (PSV40- | SCS (ScbR- | SCB1 | D | DR |
| | OscbR8) | KRAB) | | | |
| TET- | OtetO-PHPRT | tTS-H4 (TetR- | Doxycycline | D | DR |
| ON<comma> | | HDAC4) | | | |
| repressor-based | | | | | |
| T-REX | PTetO (PhCMV- | TetR | Tetracycline | D | DR |
| | OtetO2) | | | | |
| UREX | PUREX8 (PSV40- | mUTS (KRAB- | Uric acid | D | DR |
| | OhucO8) | HucR) | | | |
| VAC-ON | PVanON8 (PhCMV- | VanA4 (VanR- | Vanillic acid | D | DR |
| | OVanO8) | KRAB) | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | Tetracycline | DD | DADR |
| ON(NOT IF gate) | PhCMVmin | | Erythromycin | | |
| TET-OFFPIP- | OtetO7-OPIR3- | tTAPIT3E-KRAB | Tetracycline | DDD | DADRDR |
| ONE-ON | OETR8-PhCMVmin | | Pristinamycin | | |
| | | | IErythromycin | | |

TABLE 5
Exemplary Components of Inducible Promoters

| Name | DNA SEQUENCE | Source |
|---|---|---|
| minimal promoter, | AGAGGGTATATAATGGAAGCTCGACTTC | EU581860.1 |
| minP | CAG (SEQ ID NO: 208) | (Promega) |
| NFKB response element | GGGAATTTCCGGGGACTTTCCGGGAATT | EU581860.1 |
| protein promoter; 5x | TCCGGGGACTTTCCGGGAATTTCC | (Promega) |
| NFKB-RE | (SEQ ID NO: 209) | |
| CREB response element | CACCAGACAGTGACGTCAGCTGCCAGAT | DQ904461.1 |
| protein promoter; 4x | CCCATGGCCGTCATACTGTGACGTCTTT | (Promega) |
| CRE | CAGACACCCCATTGACGTCAATGGGAGA | |
| | A (SEQ ID NO: 210) | |
| NFAT response element | GGAGGAAAAACTGTTTCATACAGAAGGC | DQ904462.1 |
| protein promoter; 3x | GTGGAGGAAAAACTGTTTCATACAGAAG | (Promega) |
| NFAT binding sites | GCGTGGAGGAAAAACTGTTTCATACAGA | |
| | AGGCGT (SEQ ID NO: 211) | |
| SRF response element | AGGATGTCCATATTAGGACATCTAGGAT | FJ773212.1 |
| protein promoter; 5x | GTCCATATTAGGACATCTAGGATGTCCA | (Promega) |
| SRE | TATTAGGACATCTAGGATGTCCATATTA | |
| | GGACATCTAGGATGTCCATATTAGGACA | |
| | TCT (SEQ ID NO: 212) | |
| SRF response element | AGTATGTCCATATTAGGACATCTACCAT | FJ773213.1 |
| protein promoter 2; | GTCCATATTAGGACATCTACTATGTCCA | (Promega) |
| 5x SRF-RE | TATTAGGACATCTTGTATGTCCATATTA | |
| | GGACATCTAAAATGTCCATATTAGGACA | |
| | TCT (SEQ ID NO: 213) | |
| AP1 response element | TGAGTCAGTGACTCAGTGAGTCAGTGAC | JQ858516.1 |
| protein promoter; 6x | TCAGTGAGTCAGTGACTCAG (SEQ ID | (Promega) |
| AP1-RE | NO: 214) | |
| TCF-LEF response | AGATCAAAGGGTTTAAGATCAAAGGGCT | JX099537.1 |
| element promoter; 8x | TAAGATCAAAGGGTATAAGATCAAAGGG | (Promega) |
| TCF-LEF-RE | CCTAAGATCAAAGGGACTAAGATCAAAG | |
| | GGTTTAAGATCAAAGGGCTTAAGATCAA | |
| | AGGGCCTA (SEQ ID NO: 215) | |
| SBEx4 | GTCTAGACGTCTAGACGTCTAGACGTCT | Addgene Cat |
| | AGAC (SEQ ID NO: 216) | No: 16495 |
| SMAD2/3 - CAGACA x4 | CAGACACAGACACAGACACAGACA | Jonk et al. |
| | (SEQ ID NO: 217) | (J Biol Chem. |
| | | 1998 Aug. 14; |
| | | 273(33): |
| | | 21145-52. |
| STAT3 binding site | Ggatccggtactcgagatctgcgatcta | Addgene |
| | agtaagcttggcattccggtactgttgg | Sequencing |
| | taaagccac (SEQ ID NO: 218) | Result |
| | | #211335 |
| 5x NFAT | GGGACTTTCCACTGGGGACTTTCCACTG | |
| | GGGACTTTCCACTGGGGACTTTCCACTG | |
| | GGGACTTTCC (SEQ ID NO: 219) | |
| min AdeP | AGACGCTAGCGGGGGGCTATAAAAGGGG | |
| | GTGGGGGCGTTCGTCCTCACTCT (SEQ | |
| | ID NO: 220) | |
| 5x NFAT minAdeP | GGGACTTTCCACTGGGGACTTTCCACTG | |
| | GGGACTTTCCACTGGGGACTTTCCACTG | |
| | GGGACTTTCCACTCCTGCAGGagctGGC | |
| | GCGCCAGACGCTAGCGGGGGGCTATAAA | |
| | AGGGGGTGGGGGCGTTCGTCCTCACTCT | |
| | (SEQ ID NO: 221) | |
| YB-TATA | TCTAGAGGGTATATAATGGGGGCCA | |
| | (SEQ ID NO: 222) | |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbQ) promoter. In some embodiments, the promoter is a constitutive promoter. Exemplary constitutive promoters are shown in Table 6.

TABLE 6
Exemplary Constitutive Promoters

| Name | DNA SEQUENCE |
|---|---|
| CMV | GTTGACATTGATTATTGACTAGTTATTAATAGTAATCAATTACGGGGTCATTAGT |
| | TCATAGCCCATATATGGAGTTCCGCGTTACATAACTTACGGTAAATGGCCCGCCT |
| | GGCTGACCGCCCAACGACCCCCGCCCATTGACGTCAATAATGACGTATGTTCCCA |
| | TAGTAACGCCAATAGGGACTTTCCATTGACGTCAATGGGTGGAGTATTTACGGTA |
| | AACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTAT |
| | GGGACTTTCCTACTTGGCAGTACATCTACGTATTAGTCATCGCTATTACCATGGT |
| | GATGCGGTTTTGGCAGTACATCAATGGGCGTGGATAGCGGTTTGACTCACGGGGA |
| | TTTCCAAGTCTCCACCCCATTGACGTCAATGGGAGTTTGTTTTGGCACCAAAATC |
| | AACGGGACTTTCCAAAATGTCGTAACAACTCCGCCCCATTGACGCAAATGGGCGG |
| | TAGGCGTGTACGGTGGGAGGTCTATATAAGCAGAGCTC (SEQ ID NO: 223) |
| EF1a | GGCTCCGGTGCCCGTCAGTGGGCAGAGCGCACATCGCCCACAGTCCCCGAGAAGT |
| | TGGGGGGAGGGGTCGGCAATTGAACCGGTGCCTAGAGAAGGTGGCGCGGGGTAAA |
| | CTGGGAAAGTGATGCCGTGTACTGGCTCCGCCTTTTTCCCGAGGGTGGGGGAGAA |
| | CCGTATATAAGTGCAGTAGTCGCCGTGAACGTTCTTTTTCGCAACGGGTTTGCCG |
| | CCAGAACACAGGTAAGTGCCGTGTGTGGTTCCCGCGGGCCTGGCCTCTTTACGGG |
| | TTATGGCCCTTGCGTGCCTTGAATTACTTCCACCTGGCTGCAGTACGTGATTCTT |
| | GATCCCGAGCTTCGGGTTGGAAGTGGGTGGGAGAGTTCGAGGCCTTGCGCTTAAG |
| | GAGCCCCTTCGCCTCGTGCTTGAGTTGAGGCCTGGCCTGGGCGCTGGGGCCGCCG |
| | CGTGCGAATCTGGTGGCACCTTCGCGCCTGTCTCGCTGCTTTCGATAAGTCTCTA |
| | GCCATTTAAAATTTTTGATGACCTGCTGCGACGCTTTTTTTCTGGCAAGATAGTC |
| | TTGTAAATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCCTGCGA |
| | GCGCGACCACCGAGAATCGGACGGGGGTAGTCTCAAGCTGGCCGGCCTGCTCTGG |
| | TGCCTGTCCTCGCGCCGCCGTGTATCGCCCCGCCCCGGGCGGCAAGGCTGGCCCG |
| | GTCGGCACCAGTTGCGTGAGCGGAAAGATGGCCGCTTCCCGGTCCTGCTGCAGGG |
| | AGCTCAAAATGGAGGACGCGGCGCTCGGGAGAGCGGGCGGGTGAGTCACCCACAC |
| | AAAGGAAAAGGGCCTTTCCGTCCTCAGCCGTCGCTTCATGTGACTCCACGGAGTA |
| | CCGGGCGCCGTCCAGGCACCTCGATTAGTTCTCGAGCTTTTGGAGTACGTCGTCT |
| | TTAGGTTGGGGGGAGGGGTTTTATGCGATGGAGTTTCCCCACACTGAGTGGGTGG |
| | AGACTGAAGTTAGGCCAGCTTGGCACTTGATGTAATTCTCCTTGGAATTTGCCCT |
| | TTTTGAGTTTGGATCTTGGTTCATTCTCAAGCCTCAGACAGTGGTTCAAAGTTTT |
| | TTTCTTCCATTTCAGGTGTCGTGA (SEQ ID NO: 224) |
| EFS | GGATCTGCGATCGCTCCGGTGCCCGTCAGTGGGCAGAGCGCACATCGCCCACAGT |
| | CCCCGAGAAGTTGGGGGGAGGGGTCGGCAATTGAACCGGTGCCTAGAGAAGGTGG |
| | CGCGGGGTAAACTGGGAAAGTGATGTCGTGTACTGGCTCCGCCTTTTTCCCGAGG |
| | GTGGGGGAGAACCGTATATAAGTGCAGTAGTCGCCGTGAACGTTCTTTTTCGCAA |
| | CGGGTTTGCCGCCAGAACACAGCTGAAGCTTCGAGGGGCTCGCATCTCTCCTTCA |
| | CGCGCCCGCCGCCCTACCTGAGGCCGCCATCCACGCCGGTTGAGTCGCGTTCTGC |
| | CGCCTCCCGCCTGTGGTGCCTCCTGAACTGCGTCCGCCGTCTAGGTAAGTTTAAA |
| | GCTCAGGTCGAGACCGGGCCTTTGTCCGGCGCTCCCTTGGAGCCTACCTAGACTC |
| | AGCCGGCTCTCCACGCTTTGCCTGACCCTGCTTGCTCAACTCTACGTCTTTGTTT |
| | CGTTTTCTGTTCTGCGCCGTTACAGATCCAAGCTGTGACCGGCGCCTAC (SEQ |
| | ID NO: 225) |
| MND | TTTATTTAGTCTCCAGAAAAAGGGGGGAATGAAAGACCCCACCTGTAGGTTTGGC |
| | AAGCTAGGATCAAGGTTAGGAACAGAGAGACAGCAGAATATGGGCCAAACAGGAT |
| | ATCTGTGGTAAGCAGTTCCTGCCCCGGCTCAGGGCCAAGAACAGTTGGAACAGCA |
| | GAATATGGGCCAAACAGGATATCTGTGGTAAGCAGTTCCTGCCCCGGCTCAGGGC |
| | CAAGAACAGATGGTCCCCAGATGCGGTCCCGCCCTCAGCAGTTTCTAGAGAACCA |
| | TCAGATGTTTCCAGGGTGCCCCAAGGACCTGAAATGACCCTGTGCCTTATTTGAA |
| | CTAACCAATCAGTTCGCTTCTCGCTTCTGTTCGCGCGCTTCTGCTCCCCGAGCTC |
| | AATAAAAGAGCCCA (SEQ ID NO: 226) |
| PGK | GGGGTTGGGGTTGCGCCTTTTCCAAGGCAGCCCTGGGTTTGCGCAGGGACGCGGC |
| | TGCTCTGGGCGTGGTTCCGGGAAACGCAGCGGCGCCGACCCTGGGTCTCGCACAT |
| | TCTTCACGTCCGTTCGCAGCGTCACCCGGATCTTCGCCGCTACCCTTGTGGGCCC |
| | CCCGGCGACGCTTCCTGCTCCGCCCCTAAGTCGGGAAGGTTCCTTGCGGTTCGCG |
| | GCGTGCCGGACGTGACAAACGGAAGCCGCACGTCTCACTAGTACCCTCGCAGACG |
| | GACAGCGCCAGGGAGCAATGGCAGCGCGCCGACCGCGATGGGCTGTGGCCAATAG |
| | CGGCTGCTCAGCGGGGCGCGCCGAGAGCAGCGGCCGGGAAGGGGCGGTGCGGGAG |
| | GCGGGGTGTGGGGCGGTAGTGTGGGCCCTGTTCCTGCCCGCGCGGTGTTCCGCAT |
| | TCTGCAAGCCTCCGGAGCGCACGTCGGCAGTCGGCTCCCTCGTTGACCGAATCAC |
| | CGACCTCTCTCCCCAG (SEQ ID NO: 227) |
| SFFV | GTAACGCCATTTTGCAAGGCATGGAAAAATACCAAACCAAGAATAGAGAAGTTCA |
| | GATCAAGGGCGGGTACATGAAAATAGCTAACGTTGGGCCAAACAGGATATCTGCG |
| | GTGAGCAGTTTCGGCCCCGGCCCGGGGCCAAGAACAGATGGTCACCGCAGTTTCG |
| | GCCCCGGCCCGAGGCCAAGAACAGATGGTCCCCAGATATGGCCCAACCCTCAGCA |
| | GTTTCTTAAGACCCATCAGATGTTTCCAGGCTCCCCCAAGGACCTGAAATGACCC |
| | TGCGCCTTATTTGAATTAACCAATCAGCCTGCTTCTCGCTTCTGTTCGCGCGCTT |
| | CTGCTTCCCGAGCTCTATAAAAGAGCTCACAACCCCTCACTCGGCGCGCCAGTCC |
| | TCCGACAGACTGAGTCGCCCGGG (SEQ ID NO: 228) |
| SV40 | CTGTGGAATGTGTGTCAGTTAGGGTGTGGAAAGTCCCCAGGCTCCCCAGCAGGCA |
| | GAAGTATGCAAAGCATGCATCTCAATTAGTCAGCAACCAGGTGTGGAAAGTCCCC |
| | AGGCTCCCCAGCAGGCAGAAGTATGCAAAGCATGCATCTCAATTAGTCAGCAACC |
| | ATAGTCCCGCCCCTAACTCCGCCCATCCCGCCCCTAACTCCGCCCAGTTCCGCCC |
| | ATTCTCCGCCCCATGGCTGACTAATTTTTTTTATTTATGCAGAGGCCGAGGCCGC |
| | CTCTGCCTCTGAGCTATTCCAGAAGTAGTGAGGAGGCTTTTTTGGAGGCCTAGGC |
| | TTTTGCAAAAAGCT (SEQ ID NO: 229) |
| UbC | GCGCCGGGTTTTGGCGCCTCCCGCGGGCGCCCCCCTCCTCACGGCGAGCGCTGCC |
| | ACGTCAGACGAAGGGCGCAGGAGCGTTCCTGATCCTTCCGCCCGGACGCTCAGGA |
| | CAGCGGCCCGCTGCTCATAAGACTCGGCCTTAGAACCCCAGTATCAGCAGAAGGA |
| | CATTTTAGGACGGGACTTGGGTGACTCTAGGGCACTGGTTTTCTTTCCAGAGAGC |
| | GGAACAGGCGAGGAAAAGTAGTCCCTTCTCGGCGATTCTGCGGAGGGATCTCCGT |
| | GGGGCGGTGAACGCCGATGATTATATAAGGACGCGCCGGGTGTGGCACAGCTAGT |
| | TCCGTCGCAGCCGGGATTTGGGTCGCGGTTCTTGTTTGTGGATCGCTGTGATCGT |
| | CACTTGGTGAGTTGCGGGCTGCTGGGCTGGCCGGGGCTTTCGTGGCCGCCGGGCC |
| | GCTCGGTGGGACGGAAGCGTGTGGAGAGACCGCCAAGGGCTGTAGTCTGGGTCCG |
| | CGAGCAAGGTTGCCCTGAACTGGGGGTTGGGGGGAGCGCACAAAATGGCGGCTGT |
| | TCCCGAGTCTTGAATGGAAGACGCTTGTAAGGCGGGCTGTGAGGTCGTTGAAACA |
| | AGGTGGGGGGCATGGTGGGCGGCAAGAACCCAAGGTCTTGAGGCCTTCGCTAATG |
| | CGGGAAAGCTCTTATTCGGGTGAGATGGGCTGGGGCACCATCTGGGGACCCTGAC |
| | GTGAAGTTTGTCACTGACTGGAGAACTCGGGTTTGTCGTCTGGTTGCGGGGGCGG |
| | CAGTTATGCGGTGCCGTTGGGCAGTGCACCCGTACCTTTGGGAGCGCGCGCCTCG |
| | TCGTGTCGTGACGTCACCCGTTCTGTTGGCTTATAATGCAGGGTGGGGCCACCTG |
| | CCGGTAGGTGTGCGGTAGGCTTTTCTCCGTCGCAGGACGCAGGGTTCGGGCCTAG |
| | GGTAGGCTCTCCTGAATCGACAGGCGCCGGACCTCTGGTGAGGGGAGGGATAAGT |
| | GAGGCGTCAGTTTCTTTGGTCGGTTTTATGTACCTATCTTCTTAAGTAGCTGAAG |
| | CTCCGGTTTTGAACTATGCGCTCGGGGTTGGCGAGTGTGTTTTGTGAAGTTTTTT |
| | AGGCACCTTTTGAAATGTAATCATTTGGGTCAATATGTAATTTTCAGTGTTAGAC |
| | TAGTAAAGCTTCTGCAGGTCGACTCTAGAAAATTGTCCGCTAAATTCTGGCCGTT |
| | TTTGGCTTTTTTGTTAGAC (SEQ ID NO: 230) |
| hEF1aV1 | GGCTCCGGTGCCCGTCAGTGGGCAGAGCGCACATCGCCCACAGTCCCCGAGAAGT |
| | TGGGGGGAGGGGTCGGCAATTGAACCGGTGCCTAGAGAAGGTGGCGCGGGGTAAA |
| | CTGGGAAAGTGATGTCGTGTACTGGCTCCGCCTTTTTCCCGAGGGTGGGGGAGAA |
| | CCGTATATAAGTGCAGTAGTCGCCGTGAACGTTCTTTTTCGCAACGGGTTTGCCG |
| | CCAGAACACAGGTAAGTGCCGTGTGTGGTTCCCGCGGGCCTGGCCTCTTTACGGG |
| | TTATGGCCCTTGCGTGCCTTGAATTACTTCCACCTGGCTGCAGTACGTGATTCTT |
| | GATCCCGAGCTTCGGGTTGGAAGTGGGTGGGAGAGTTCGAGGCCTTGCGCTTAAG |
| | GAGCCCCTTCGCCTCGTGCTTGAGTTGAGGCCTGGCCTGGGCGCTGGGGCCGCCG |
| | CGTGCGAATCTGGTGGCACCTTCGCGCCTGTCTCGCTGCTTTCGATAAGTCTCTA |
| | GCCATTTAAAATTTTTGATGACCTGCTGCGACGCTTTTTTTCTGGCAAGATAGTC |
| | TTGTAAATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCCTGCGA |
| | GCGCGGCCACCGAGAATCGGACGGGGGTAGTCTCAAGCTGGCCGGCCTGCTCTGG |
| | TGCCTGGTCTCGCGCCGCCGTGTATCGCCCCGCCCTGGGCGGCAAGGCTGGCCCG |
| | GTCGGCACCAGTTGCGTGAGCGGAAAGATGGCCGCTTCCCGGCCCTGCTGCAGGG |
| | AGCTCAAAATGGAGGACGCGGCGCTCGGGAGAGCGGGCGGGTGAGTCACCCACAC |
| | AAAGGAAAAGGGCCTTTCCGTCCTCAGCCGTCGCTTCATGTGACTCCACGGAGTA |
| | CCGGGCGCCGTCCAGGCACCTCGATTAGTTCTCGAGCTTTTGGAGTACGTCGTCT |
| | TTAGGTTGGGGGGAGGGGTTTTATGCGATGGAGTTTCCCCACACTGAGTGGGTGG |
| | AGACTGAAGTTAGGCCAGCTTGGCACTTGATGTAATTCTCCTTGGAATTTGCCCT |
| | TTTTGAGTTTGGATCTTGGTTCATTCTCAAGCCTCAGACAGTGGTTCAAAGTTTT |
| | TTTCTTCCATTTCAGGTGTCGTGA (SEQ ID NO: 231) |
| hCAGG | ACTAGTTATTAATAGTAATCAATTACGGGGTCATTAGTTCATAGCCCATATATGG |
| | AGTTCCGCGTTACATAACTTACGGTAAATGGCCCGCCTGGCTGACCGCCCAACGA |
| | CCCCCGCCCATTGACGTCAATAATGACGTATGTTCCCATAGTAACGCCAATAGGG |
| | ACTTTCCATTGACGTCAATGGGTGGAGTATTTACGGTAAACTGCCCACTTGGCAG |
| | TACATCAAGTGTATCATATGCCAAGTACGCCCCCTATTGACGTCAATGACGGTAA |
| | ATGGCCCGCCTGGCATTATGCCCAGTACATGACCTTATGGGACTTTCCTACTTGG |
| | CAGTACATCTACGTATTAGTCATCGCTATTACCATGGTCGAGGTGAGCCCCACGT |
| | TCTGCTTCACTCTCCCCATCTCCCCCCCCTCCCCACCCCCAATTTTGTATTTATT |
| | TATTTTTTAATTATTTTGTGCAGCGATGGGGGCGGGGGGGGGGGGGGGGCGCGCG |
| | CCAGGCGGGGCGGGGCGGGGCGAGGGGCGGGGCGGGGCGAGGCGGAGAGGTGCGG |
| | CGGCAGCCAATCAGAGCGGCGCGCTCCGAAAGTTTCCTTTTATGGCGAGGCGGCG |
| | GCGGCGGCGGCCCTATAAAAAGCGAAGCGCGCGGCGGGCGGGGAGTCGCTGCGAC |
| | GCTGCCTTCGCCCCGTGCCCCGCTCCGCCGCCGCCTCGCGCCGCCCGCCCCGGCT |
| | CTGACTGACCGCGTTACTCCCACAGGTGAGCGGGCGGGACGGCCCTTCTCCTCCG |
| | GGCTGTAATTAGCGCTTGGTTTAATGACGGCTTGTTTCTTTTCTGTGGCTGCGTG |
| | AAAGCCTTGAGGGGCTCCGGGAGGGCCCTTTGTGCGGGGGGAGCGGCTCGGGGGG |
| | TGCGTGCGTGTGTGTGTGCGTGGGGAGCGCCGCGTGCGGCTCCGCGCTGCCCGGC |
| | GGCTGTGAGCGCTGCGGGCGCGGCGCGGGGCTTTGTGCGCTCCGCAGTGTGCGCG |
| | AGGGGAGCGCGGCCGGGGGCGGTGCCCCGCGGTGCGGGGGGGGCTGCGAGGGGAA |
| | CAAAGGCTGCGTGCGGGGTGTGTGCGTGGGGGGGTGAGCAGGGGGTGTGGGCGCG |
| | TCGGTCGGGCTGCAACCCCCCCTGCACCCCCCTCCCCGAGTTGCTGAGCACGGCC |
| | CGGCTTCGGGTGCGGGGCTCCGTACGGGGCGTGGCGCGGGGCTCGCCGTGCCGGG |
| | CGGGGGGTGGCGGCAGGTGGGGGTGCCGGGCGGGGCGGGGCCGCCTCGGGCCGGG |
| | GAGGGCTCGGGGGAGGGGCGCGGCGGCCCCCGGAGCGCCGGCGGCTGTCGAGGCG |
| | CGGCGAGCCGCAGCCATTGCCTTTTATGGTAATCGTGCGAGAGGGCGCAGGGACT |
| | TCCTTTGTCCCAAATCTGTGCGGAGCCGAAATCTGGGAGGCGCCGCCGCACCCCC |
| | TCTAGCGGGCGCGGGGCGAAGCGGTGCGGCGCCGGCAGGAAGGAAATGGGCGGGG |
| | AGGGCCTTCGTGCGTCGCCGCGCCGCCGTCCCCTTCTCCCTCTCCAGCCTCGGGG |
| | CTGTCCGCGGGGGGACGGCTGCCTTCGGGGGGGACGGGGCAGGGCGGGGTTCGGC |
| | TTCTGGCGTGTGACCGGCGGCTCTAGAGCCTCTGCTAACCATGTTCATGCCTTCT |
| | TCTTTTTCCTACAGCTCCTGGGCAACGTGCTGGTTATTGTGCTGTCTCATCATTT |
| | TGGCAAAGAATTC (SEQ ID NO: 232) |
| hEF1aV2 | GGGCAGAGCGCACATCGCCCACAGTCCCCGAGAAGTTGGGGGGAGGGGTCGGCAA |
| | TTGAACCGGTGCCTAGAGAAGGTGGCGCGGGGTAAACTGGGAAAGTGATGTCGTG |
| | TACTGGCTCCGCCTTTTTCCCGAGGGTGGGGGAGAACCGTATATAAGTGCAGTAG |
| | TCGCCGTGAACGTTCTTTTTCGCAACGGGTTTGCCGCCAGAACACAG (SEQ ID |
| | NO: 233) |
| hACTb | CCACTAGTTCCATGTCCTTATATGGACTCATCTTTGCCTATTGCGACACACACTC |
| | AATGAACACCTACTACGCGCTGCAAAGAGCCCCGCAGGCCTGAGGTGCCCCCACC |
| | TCACCACTCTTCCTATTTTTGTGTAAAAATCCAGCTTCTTGTCACCACCTCCAAG |
| | GAGGGGGAGGAGGAGGAAGGCAGGTTCCTCTAGGCTGAGCCGAATGCCCCTCTGT |
| | GGTCCCACGCCACTGATCGCTGCATGCCCACCACCTGGGTACACACAGTCTGTGA |
| | TTCCCGGAGCAGAACGGACCCTGCCCACCCGGTCTTGTGTGCTACTCAGTGGACA |
| | GACCCAAGGCAAGAAAGGGTGACAAGGACAGGGTCTTCCCAGGCTGGCTTTGAGT |
| | TCCTAGCACCGCCCCGCCCCCAATCCTCTGTGGCACATGGAGTCTTGGTCCCCAG |
| | AGTCCCCCAGCGGCCTCCAGATGGTCTGGGAGGGCAGTTCAGCTGTGGCTGCGCA |
| | TAGCAGACATACAACGGACGGTGGGCCCAGACCCAGGCTGTGTAGACCCAGCCCC |
| | CCCGCCCCGCAGTGCCTAGGTCACCCACTAACGCCCCAGGCCTGGTCTTGGCTGG |
| | GCGTGACTGTTACCCTCAAAAGCAGGCAGCTCCAGGGTAAAAGGTGCCCTGCCCT |
| | GTAGAGCCCACCTTCCTTCCCAGGGCTGCGGCTGGGTAGGTTTGTAGCCTTCATC |
| | ACGGGCCACCTCCAGCCACTGGACCGCTGGCCCCTGCCCTGTCCTGGGGAGTGTG |
| | GTCCTGCGACTTCTAAGTGGCCGCAAGCCACCTGACTCCCCCAACACCACACTCT |
| | ACCTCTCAAGCCCAGGTCTCTCCCTAGTGACCCACCCAGCACATTTAGCTAGCTG |
| | AGCCCCACAGCCAGAGGTCCTCAGGCCCTGCTTTCAGGGCAGTTGCTCTGAAGTC |
| | GGCAAGGGGGAGTGACTGCCTGGCCACTCCATGCCCTCCAAGAGCTCCTTCTGCA |
| | GGAGCGTACAGAACCCAGGGCCCTGGCACCCGTGCAGACCCTGGCCCACCCCACC |
| | TGGGCGCTCAGTGCCCAAGAGATGTCCACACCTAGGATGTCCCGCGGTGGGTGGG |
| | GGGCCCGAGAGACGGGCAGGCCGGGGGCAGGCCTGGCCATGCGGGGCCGAACCGG |
| | GCACTGCCCAGCGTGGGGCGCGGGGGCCACGGCGCGCGCCCCCAGCCCCCGGGCC |
| | CAGCACCCCAAGGCGGCCAACGCCAAAACTCTCCCTCCTCCTCTTCCTCAATCTC |
| | GCTCTCGCTCTTTTTTTTTTTCGCAAAAGGAGGGGAGAGGGGGTAAAAAAATGCT |
| | GCACTGTGCGGCGAAGCCGGTGAGTGAGCGGCGCGGGGCCAATCAGCGTGCGCCG |
| | TTCCGAAAGTTGCCTTTTATGGCTCGAGCGGCCGCGGCGGCGCCCTATAAAACCC |
| | AGCGGCGCGACGCGCCACCACCGCCGAGACCGCGTCCGCCCCGCGAGCACAGAGC |
| | CTCGCCTTTGCCGATCCGCCGCCCGTCCACACCCGCCGCCAGGTAAGCCCGGCCA |
| | GCCGACCGGGGCAGGCGGCTCACGGCCCGGCCGCAGGCGGCCGCGGCCCCTTCGC |
| | CCGTGCAGAGCCGCCGTCTGGGCCGCAGCGGGGGGCGCATGGGGGGGGAACCGGA |
| | CCGCCGTGGGGGGCGCGGGAGAAGCCCCTGGGCCTCCGGAGATGGGGGACACCCC |
| | ACGCCAGTTCGGAGGCGCGAGGCCGCGCTCGGGAGGCGCGCTCCGGGGGTGCCGC |
| | TCTCGGGGCGGGGGCAACCGGCGGGGTCTTTGTCTGAGCCGGGCTCTTGCCAATG |
| | GGGATCGCAGGGTGGGCGCGGCGGAGCCCCCGCCAGGCCCGGTGGGGGCTGGGGC |
| | GCCATTGCGCGTGCGCGCTGGTCCTTTGGGCGCTAACTGCGTGCGCGCTGGGAAT |
| | TGGCGCTAATTGCGCGTGCGCGCTGGGACTCAAGGCGCTAACTGCGCGTGCGTTC |
| | TGGGGCCCGGGGTGCCGCGGCCTGGGCTGGGGCGAAGGCGGGCTCGGCCGGAAGG |
| | GGTGGGGTCGCCGCGGCTCCCGGGCGCTTGCGCGCACTTCCTGCCCGAGCCGCTG |
| | GCCGCCCGAGGGTGTGGCCGCTGCGTGCGCGCGCGCCGACCCGGCGCTGTTTGAA |
| | CCGGGCGGAGGCGGGGCTGGCGCCCGGTTGGGAGGGGGTTGGGGCCTGGCTTCCT |
| | GCCGCGCGCCGCGGGGACGCCTCCGACCAGTGTTTGCCTTTTATGGTAATAACGC |
| | GGCCGGCCCGGCTTCCTTTGTCCCCAATCTGGGCGCGCGCCGGCGCCCCCTGGCG |
| | GCCTAAGGACTCGGCGCGCCGGAAGTGGCCAGGGCGGGGGCGACCTCGGCTCACA |
| | GCGCGCCCGGCTAT (SEQ ID NO: 234) |
| heIF4A1 | GTTGATTTCCTTCATCCCTGGCACACGTCCAGGCAGTGTCGAATCCATCTCTGCT |
| | ACAGGGGAAAACAAATAACATTTGAGTCCAGTGGAGACCGGGAGCAGAAGTAAAG |
| | GGAAGTGATAACCCCCAGAGCCCGGAAGCCTCTGGAGGCTGAGACCTCGCCCCCC |
| | TTGCGTGATAGGGCCTACGGAGCCACATGACCAAGGCACTGTCGCCTCCGCACGT |
| | GTGAGAGTGCAGGGCCCCAAGATGGCTGCCAGGCCTCGAGGCCTGACTCTTCTAT |
| | GTCACTTCCGTACCGGCGAGAAAGGCGGGCCCTCCAGCCAATGAGGCTGCGGGGC |
| | GGGCCTTCACCTTGATAGGCACTCGAGTTATCCAATGGTGCCTGCGGGCCGGAGC |
| | GACTAGGAACTAACGTCATGCCGAGTTGCTGAGCGCCGGCAGGCGGGGCCGGGGC |
| | GGCCAAACCAATGCGATGGCCGGGGCGGAGTCGGGCGCTCTATAAGTTGTCGATA |
| | GGCGGGCACTCCGCCCTAGTTTCTAAGGACCATG (SEQ ID NO: 235) |
| hGAPDH | AGTTCCCCAACTTTCCCGCCTCTCAGCCTTTGAAAGAAAGAAAGGGGAGGGGGCA |
| | GGCCGCGTGCAGTCGCGAGCGGTGCTGGGCTCCGGCTCCAATTCCCCATCTCAGT |
| | CGCTCCCAAAGTCCTTCTGTTTCATCCAAGCGTGTAAGGGTCCCCGTCCTTGACT |
| | CCCTAGTGTCCTGCTGCCCACAGTCCAGTCCTGGGAACCAGCACCGATCACCTCC |
| | CATCGGGCCAATCTCAGTCCCTTCCCCCCTACGTCGGGGCCCACACGCTCGGTGC |
| | GTGCCCAGTTGAACCAGGCGGCTGCGGAAAAAAAAAAGCGGGGAGAAAGTAGGGC |
| | CCGGCTACTAGCGGTTTTACGGGCGCACGTAGCTCAGGCCTCAAGACCTTGGGCT |
| | GGGACTGGCTGAGCCTGGCGGGAGGCGGGGTCCGAGTCACCGCCTGCCGCCGCGC |
| | CCCCGGTTTCTATAAATTGAGCCCGCAGCCTCCCGCTTCGCTCTCTGCTCCTCCT |
| | GTTCGACAGTCAGCCGCATCTTCTTTTGCGTCGCCAGGTGAAGACGGGCGGAGAG |
| | AAACCCGGGAGGCTAGGGACGGCCTGAAGGCGGCAGGGGGGGGCGCAGGCCGGAT |
| | GTGTTCGCGCCGCTGCGGGGTGGGCCCGGGCGGCCTCCGCATTGCAGGGGCGGGC |
| | GGAGGACGTGATGCGGCGCGGGCTGGGCATGGAGGCCTGGTGGGGGAGGGGAGGG |
| | GAGGCGTGGGTGTCGGCCGGGGCCACTAGGCGCTCACTGTTCTCTCCCTCCGCGC |
| | AGCCGAGCCACATCGCTGAGACAC (SEQ ID NO: 236) |
| hGRP78 | AGTGCGGTTACCAGCGGAAATGCCTCGGGGTCAGAAGTCGCAGGAGAGATAGACA |
| | GCTGCTGAACCAATGGGACCAGCGGATGGGGCGGATGTTATCTACCATTGGTGAA |
| | CGTTAGAAACGAATAGCAGCCAATGAATCAGCTGGGGGGGCGGAGCAGTGACGTT |
| | TATTGCGGAGGGGGCCGCTTCGAATCGGCGGCGGCCAGCTTGGTGGCCTGGGCCA |
| | ATGAACGGCCTCCAACGAGCAGGGCCTTCACCAATCGGCGGCCTCCACGACGGGG |
| | CTGGGGGAGGGTATATAAGCCGAGTAGGCGACGGTGAGGTCGACGCCGGCCAAGA |
| | CAGCACAGACAGATTGACCTATTGGGGTGTTTCGCGAGTGTGAGAGGGAAGCGCC |
| | GCGGCCTGTATTTCTAGACCTGCCCTTCGCCTGGTTCGTGGCGCCTTGTGACCCC |
| | GGGCCCCTGCCGCCTGCAAGTCGGAAATTGCGCTGTGCTCCTGTGCTACGGCCTG |
| | TGGCTGGACTGCCTGCTGCTGCCCAACTGGCTGGCAC (SEQ ID NO: 237) |
| hGRP94 | TAGTTTCATCACCACCGCCACCCCCCCGCCCCCCCGCCATCTGAAAGGGTTCTAG |
| | GGGATTTGCAACCTCTCTCGTGTGTTTCTTCTTTCCGAGAAGCGCCGCCACACGA |
| | GAAAGCTGGCCGCGAAAGTCGTGCTGGAATCACTTCCAACGAAACCCCAGGCATA |
| | GATGGGAAAGGGTGAAGAACACGTTGCCATGGCTACCGTTTCCCCGGTCACGGAA |
| | TAAACGCTCTCTAGGATCCGGAAGTAGTTCCGCCGCGACCTCTCTAAAAGGATGG |
| | ATGTGTTCTCTGCTTACATTCATTGGACGTTTTCCCTTAGAGGCCAAGGCCGCCC |
| | AGGCAAAGGGGCGGTCCCACGCGTGAGGGGCCCGCGGAGCCATTTGATTGGAGAA |
| | AAGCTGCAAACCCTGACCAATCGGAAGGAGCCACGCTTCGGGCATCGGTCACCGC |
| | ACCTGGACAGCTCCGATTGGTGGACTTCCGCCCCCCCTCACGAATCCTCATTGGG |
| | TGCCGTGGGTGCGTGGTGCGGCGCGATTGGTGGGTTCATGTTTCCCGTCCCCCGC |
| | CCGCGAGAAGTGGGGGTGAAAAGCGGCCCGACCTGCTTGGGGTGTAGTGGGCGGA |
| | CCGCGCGGCTGGAGGTGTGAGGATCCGAACCCAGGGGTGGGGGGTGGAGGCGGCT |
| | CCTGCGATCGAAGGGGACTTGAGACTCACCGGCCGCACGTC (SEQ ID NO: |
| | 238) |
| hHSP70 | GGGCCGCCCACTCCCCCTTCCTCTCAGGGTCCCTGTCCCCTCCAGTGAATCCCAG |
| | AAGACTCTGGAGAGTTCTGAGCAGGGGGCGGCACTCTGGCCTCTGATTGGTCCAA |
| | GGAAGGCTGGGGGGCAGGACGGGAGGCGAAAACCCTGGAATATTCCCGACCTGGC |
| | AGCCTCATCGAGCTCGGTGATTGGCTCAGAAGGGAAAAGGCGGGTCTCCGTGACG |
| | ACTTATAAAAGCCCAGGGGCAAGCGGTCCGGATAACGGCTAGCCTGAGGAGCTGC |
| | TGCGACAGTCCACTACCTTTTTCGAGAGTGACTCCCGTTGTCCCAAGGCTTCCCA |
| | GAGCGAACCTGTGCGGCTGCAGGCACCGGCGCGTCGAGTTTCCGGCGTCCGGAAG |
| | GACCGAGCTCTTCTCGCGGATCCAGTGTTCCGTTTCCAGCCCCCAATCTCAGAGC |
| | GGAGCCGACAGAGAGCAGGGAACCC (SEQ ID NO: 239) |
| hKINb | GCCCCACCCCCGTCCGCGTTACAACCGGGAGGCCCGCTGGGTCCTGCACCGTCAC |
| | CCTCCTCCCTGTGACCGCCCACCTGATACCCAAACAACTTTCTCGCCCCTCCAGT |
| | CCCCAGCTCGCCGAGCGCTTGCGGGGAGCCACCCAGCCTCAGTTTCCCCAGCCCC |
| | GGGCGGGGCGAGGGGCGATGACGTCATGCCGGCGCGCGGCATTGTGGGGCGGGGC |
| | GAGGCGGGGCGCCGGGGGGAGCAACACTGAGACGCCATTTTCGGCGGCGGGAGCG |
| | GCGCAGGCGGCCGAGCGGGACTGGCTGGGTCGGCTGGGCTGCTGGTGCGAGGAGC |
| | CGCGGGGCTGTGCTCGGCGGCCAAGGGGACAGCGCGTGGGTGGCCGAGGATGCTG |
| | CGGGGCGGTAGCTCCGGCGCCCCTCGCTGGTGACTGCTGCGCCGTGCCTCACACA |
| | GCCGAGGCGGGCTCGGCGCACAGTCGCTGCTCCGCGCTCGCGCCCGGCGGCGCTC |
| | CAGGTGCTGACAGCGCGAGAGAGCGCGGCCTCAGGAGCAACAC (SEQ ID NO: |
| | 240) |
| hUBIb | TTCCAGAGCTTTCGAGGAAGGTTTCTTCAACTCAAATTCATCCGCCTGATAATTT |
| | TCTTATATTTTCCTAAAGAAGGAAGAGAAGCGCATAGAGGAGAAGGGAAATAATT |
| | TTTTAGGAGCCTTTCTTACGGCTATGAGGAATTTGGGGCTCAGTTGAAAAGCCTA |
| | AACTGCCTCTCGGGAGGTTGGGCGCGGCGAACTACTTTCAGCGGCGCACGGAGAC |
| | GGCGTCTACGTGAGGGGTGATAAGTGACGCAACACTCGTTGCATAAATTTGCGCT |
| | CCGCCAGCCCGGAGCATTTAGGGGCGGTTGGCTTTGTTGGGTGAGCTTGTTTGTG |
| | TCCCTGTGGGTGGACGTGGTTGGTGATTGGCAGGATCCTGGTATCCGCTAACAGG |
| | TACTGGCCCACAGCCGTAAAGACCTGCGGGGGCGTGAGAGGGGGGAATGGGTGAG |
| | GTCAAGCTGGAGGCTTCTTGGGGTTGGGTGGGCCGCTGAGGGGAGGGGAGGGCGA |
| | GGTGACGCGACACCCGGCCTTTCTGGGAGAGTGGGCCTTGTTGACCTAAGGGGGG |
| | CGAGGGCAGTTGGCACGCGCACGCGCCGACAGAAACTAACAGACATTAACCAACA |
| | GCGATTCCGTCGCGTTTACTTGGGAGGAAGGCGGAAAAGAGGTAGTTTGTGTGGC |
| | TTCTGGAAACCCTAAATTTGGAATCCCAGTATGAGAATGGTGTCCCTTCTTGTGT |
| | TTCAATGGGATTTTTACTTCGCGAGTCTTGTGGGTTTGGTTTTGTTTTCAGTTTG |
| | CCTAACACCGTGCTTAGGTTTGAGGCAGATTGGAGTTCGGTCGGGGGAGTTTGAA |
| | TATCCGGAACAGTTAGTGGGGAAAGCTGTGGACGCTTGGTAAGAGAGCGCTCTGG |
| | ATTTTCCGCTGTTGACGTTGAAACCTTGAATGACGAATTTCGTATTAAGTGACTT |
| | AGCCTTGTAAAATTGAGGGGAGGCTTGCGGAATATTAACGTATTTAAGGCATTTT |
| | GAAGGAATAGTTGCTAATTTTGAAGAATATTAGGTGTAAAAGCAAGAAATACAAT |
| | GATCCTGAGGTGACACGCTTATGTTTTACTTTTAAACTAGGTCACC (SEQ ID |
| | NO: 241) |

In some embodiments, the promoter sequence is derived from a promoter selected from: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB_TATA, minTK, inducer molecule responsive promoters, and tandem repeats thereof.

In some embodiments, the first promoter is a constitutive promoter, an inducible promoter, or a synthetic promoter. In some embodiments, the constitutive promoter is selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

Multicistronic and Multiple Promoter Systems

In some embodiments, engineered nucleic acids of the present disclosure can be multicistronic, i.e. more than one separate polypeptide (e.g., multiple exogenous polynucleotides or GPC3 CARs) can be produced from a single mRNA transcript. Engineered nucleic acids can be multicistronic through the use of various linkers, e.g., a polynucleotide sequence encoding an exogenous polynucleotide or GPC3 CAR can be linked to a nucleotide sequence encoding a second exogenous polynucleotide, such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third immunomodulating effector molecule, each separated by linkers such that separate polypeptides encoded by the first, second, and third immunomodulating effector molecules are produced).

“Linkers,” as used herein can refer to polypeptides that link a first polypeptide sequence and a second polypeptide sequence or the multicistronic linkers described above.

Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

Engineered Cells

Also provided herein are cells, and methods of producing cells, that comprise one or more engineered nucleic acids of the present disclosure. These cells are referred to herein as “engineered cells.” These cells, which typically contain one or more engineered nucleic acids, do not occur in nature. In some embodiments, the cells are isolated cells that recombinantly express the one or more engineered nucleic acids. In some embodiments, the engineered one or more nucleic acids are expressed from one or more vectors or a selected locus from the genome of the cell. In some embodiments, the cells are engineered to include a nucleic acid comprising a promoter operable linked to a nucleotide sequence encoding a GPC3-specific CAR expressing any of the peptide sequences listed in Table 1.

An engineered cell of the present disclosure can comprise an engineered nucleic acid integrated into the cell's genome. An engineered cell can comprise an engineered nucleic acid capable of expression without integrating into the cell's genome, for example, engineered with a transient expression system such as a plasmid or mRNA.

In some embodiments, the engineered cells further express one or more immunomodulating effectors.

Any suitable immunomodulating effector molecule known in the art can be encoded by the engineered nucleic acid or expressed by the engineered cell. Suitable immunomodulating effector molecules can be grouped into therapeutic classes based on structure similarity, sequence similarity, or function. Immunomodulating effector molecule therapeutic classes include, but are not limited to, cytokines, chemokines, homing molecules, growth factors, co-activation molecules, tumor microenvironment modifiers, receptors, ligands, antibodies, polynucleotides, peptides, and enzymes.

In some embodiments, each immunomodulating effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, an immunomodulating effector molecule is a chemokine. Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. Non-limiting examples of chemokines that may be encoded by the engineered nucleic acids of the present disclosure include: CCL21a, CXCL10, CXCL11, CXCL13, a CXCL10-CXCL11 fusion protein, CCL19, CXCL9, and XCL1, or any combination thereof. In some embodiments, the chemokine is selected from: CCL21a, CXCL10, CXCL11, CXCL13, a CXCL10-CXCL11 fusion protein, CCL19, CXCL9, and XCL1.

In some embodiments, an immunomodulating effector molecule is a cytokine. Non-limiting examples of cytokines that may be encoded by the engineered nucleic acids of the present disclosure include: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha, or any combination thereof. In some embodiments, the cytokine is selected from: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, engineered nucleic acids are configured to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, PDGFR, anti-integrin alpha4, beta7; anti-MAdCAM; CCR9; CXCR4; SDF1; MMP-2; CXCR1; CXCR7; CCR2; CCR4; and GPR15, or any combination thereof. In some embodiments, the homing molecule is selected from: anti-integrin alpha4, beta7; anti-MAdCAM; CCR9; CXCR4; SDF1; MMP-2; CXCR1; CXCR7; CCR2; CCR4; and GPR15.

In some embodiments, engineered nucleic acids are configured to produce at least one growth factor. Suitable growth factors for use as an immunomodulating effector molecule include, but are not limited to, FLT3L and GM-CSF, or any combination thereof. In some embodiments, the growth factor is selected from: FLT3L and GM-CSF.

In some embodiments, engineered nucleic acids are configured to produce at least one co-activation molecule. Suitable co-activation molecules for use as an immunomodulating effector molecule include, but are not limited to, c-Jun, 4-1BBL and CD40L, or any combination thereof. In some embodiments, the co-activation molecule is selected from: c-Jun, 4-1BBL and CD40L.

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007). Suitable tumor microenvironment modifiers for use as an immunomodulating effector molecule include, but are not limited to, adenosine deaminase, TGFbeta inhibitors, immune checkpoint inhibitors, VEGF inhibitors, and HPGE2, or any combination thereof. In some embodiments, the tumor microenvironment modifier is selected from: adenosine deaminase, TGFbeta inhibitors, immune checkpoint inhibitors, VEGF inhibitors, and HPGE2.

In some embodiments, engineered nucleic acids are configured to produce at least one TGFbeta inhibitor. Suitable TGFbeta inhibitors for use as an immunomodulating effector molecule include, but are not limited to, an anti-TGFbeta peptide, an anti-TGFbeta antibody, a TGFb-TRAP, or combinations thereof. In some embodiments, the TGFbeta inhibitors are selected from: an anti-TGFbeta peptide, an anti-TGFbeta antibody, a TGFb-TRAP, and combinations thereof.

In some embodiments, engineered nucleic acids are configured to produce at least one immune checkpoint inhibitor. Suitable immune checkpoint inhibitors for use as an immunomodulating effector molecule include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies, or any combination thereof. In some embodiments, the immune checkpoint inhibitors are selected from: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies.

Illustrative immune checkpoint inhibitors include pembrolizumab (anti-PD-1; MK-3475/Keytruda®-Merck), nivolumamb (anti-PD-1; Opdivo®-BMS), pidilizumab (anti-PD-1 antibody; CT-011-Teva/CureTech), AMP224 (anti-PD-1; NCI), avelumab (anti-PD-L 1; Bavencio®-Pfizer), durvalumab (anti-PD-L1; MEDI4736/Imfinzi®-Medimmune/AstraZeneca), atezolizumab (anti-PD-L1; Tecentriq®-Roche/Genentech), BMS-936559 (anti-PD-L1-BMS), tremelimumab (anti-CTLA-4; Medimmune/AstraZeneca), ipilimumab (anti-CTLA-4; Yervoy®-BMS), lirilumab (anti-KIR; BMS), monalizumab (anti-NKG2A; Innate Pharma/AstraZeneca).

In some embodiments, engineered nucleic acids are configured to produce at least one VEGF inhibitor. Suitable VEGF inhibitors for use as an immunomodulating effector molecule include, but are not limited to, anti-VEGF antibodies, anti-VEGF peptides, or combinations thereof. In some embodiments, the VEGF inhibitors comprise anti-VEGF antibodies, anti-VEGF peptides, or combinations thereof.

In some embodiments, each immunomodulating effector molecule is a human-derived immunomodulating effector molecule.

In some embodiments, one or more immunomodulating effector molecules comprise a secretion signal peptide (also referred to as a signal peptide or signal sequence) at the immunomodulating effector molecule's N-terminus that direct newly synthesized proteins destined for secretion or membrane insertion to the proper protein processing pathways. In embodiments with two or more immunomodulating effector molecules, each immunomodulating effector molecule can comprise a secretion signal (S). In embodiments with two or more immunomodulating effector molecules, each immunomodulating effector molecule can comprise a secretion signal such that each immunomodulating effector molecule is secreted from an engineered cell. In embodiments, the second expression cassette comprising one or more units of (L-E)ₓ further comprises a polynucleotide sequence encoding a secretion signal peptide (S). In embodiments, for each X the corresponding secretion signal peptide is operably associated with the immunomodulating effector molecule. In embodiments, the second expression cassette comprising an ACP-responsive promoter and a second exogenous polynucleotide sequence having the formula: (L-S-E)ₓ.

The secretion signal peptide operably associated with an immunomodulating effector molecule can be a native secretion signal peptide native secretion signal peptide (e.g., the secretion signal peptide generally endogenously associated with the given immunomodulating effector molecule). The secretion signal peptide operably associated with an immunomodulating effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 7.

TABLE 7
Exemplary Signal Secretion Peptides

| | Protein | Source | |
|---|---|---|---|
| Name | SEQUENCE | (Uniprot) | DNA SEQUENCE |
| IL-12 | MCHQQLVISWFSL | P29460 | ATGTGTCACCAGCAGCTCGTTAT |
| | VFLASPLVA | | ATCCTGGTTTAGTTTGGTGTTTC |
| | (SEQ ID NO: | | TCGCTTCACCCCTGGTGGCA |
| | 245) | | (SEQ ID NO: 246) |
| IL-12 (Codon | MCHQQLVISWFSL | — | ATGTGCCATCAGCAACTCGTCAT |
| Optimized) | VFLASPLVA | | CTCCTGGTTCTCCCTTGTGTTCC |
| | (SEQ ID NO: | | TCGCTTCCCCTCTGGTCGCC |
| | 247) | | (SEQ ID NO: 248) |
| IL-2 | MQLLSCIALILAL | — | ATGCAACTGCTGTCATGTATCGC |
| (Optimized) | V (SEQ ID NO: | | ACTCATCCTGGCGCTGGTA |
| | 249) | | (SEQ ID NO: 250) |
| IL-2 (Native) | MYRMQLLSCIALS | P60568 | ATGTATCGGATGCAACTTTTGAG |
| | LALVINS (SEQ | | CTGCATCGCATTGTCTCTGGCGC |
| | ID NO: 251) | | TGGTGACAAATTCC (SEQ ID |
| | | | NO: 252) |
| Trypsinogen-2 | MNLLLILTFVAAA | P07478 | ATGAATCTCTTGCTCATACTTAC |
| | VA (SEQ ID | | GTTTGTCGCTGCTGCCGTTGCG |
| | NO: 253) | | (SEQ ID NO: 254) |
| Gaussia | MGVKVLFALICIA | — | ATGGGCGTGAAGGTCTTGTTTGC |
| Luciferase | VAEA (SEQ ID | | CCTTATCTGCATAGCTGTTGCGG |
| | NO: 255) | | AGGCG (SEQ ID NO: 256) |
| CD5 | MPMGSLQPLATLY | P06127 | ATGCCGATGGGGAGCCTTCAACC |
| | LLGMLVASCLG | | TTTGGCAACGCTTTATCTTCTGG |
| | (SEQ ID NO: | | GGATGTTGGTTGCTAGTTGCCTT |
| | 257) | | GGG (SEQ ID NO: 258) |
| IgKVII (mouse) | METDTLLLWVLLL | | ATGGAAACTGACACGTTGTTGCT |
| | WVPGSTGD (SEQ | | GTGGGTATTGCTCTTGTGGGTCC |
| | ID NO: 259) | | CAGGATCTACGGGCGAC (SEQ |
| | | | ID NO: 260) |
| IgKVII (human) | MDMRVPAQLLGLL | P01597 | ATGGATATGAGGGTTCCCGCCCA |
| | LLWLRGARC | | GCTTTTGGGGCTGCTTTTGTTGT |
| | (SEQ ID NO: | | GGCTTCGAGGGGCTCGGTGT |
| | 261) | | (SEQ ID NO: 262) |
| VSV-G | MKCLLYLAFLFIG | — | ATGAAGTGTCTGTTGTACCTGGC |
| | VNC (SEQ ID | | GTTTCTGTTCATTGGTGTAAACT |
| | NO: 263) | | GT (SEQ ID NO: 264) |
| Prolactin | MNIKGSPWKGSLL | P01236 | ATGAATATCAAAGGAAGTCCGTG |
| | LLLVSNLLLCQSV | | GAAGGGTAGTCTCCTGCTGCTCC |
| | AP (SEQ ID | | TCGTATCTAACCTTCTCCTTTGT |
| | NO: 265) | | CAATCCGTGGCACCC (SEQ ID |
| | | | NO: 266) |
| Serum albumin | MKWVTFISLLFLF | P02768 | ATGAAATGGGTAACATTCATATC |
| preproprotein | SSAYS (SEQ ID | | ACTTCTCTTTCTGTTCAGCTCTG |
| | NO: 267) | | CGTATTCT (SEQ ID NO: |
| | | | 268) |
| Azurocidin | MTRLTVLALLAGL | 20160 | ATGACAAGGCTTACTGTTTTGGC |
| preproprotein | LASSRA (SEQ | | TCTCCTCGCTGGACTCTTGGCTT |
| | ID NO: 269) | | CCTCCCGAGCA (SEQ ID NO: |
| | | | 270) |
| Osteonectin | MRAWIFFLLCLAG | P09486 | ATGAGGGCTTGGATTTTTTTTCT |
| (BM40) | RALA (SEQ ID | | GCTCTGCCTTGCCGGTCGAGCCC |
| | NO: 271) | | TGGCG (SEQ ID NO: 272) |
| CD33 | MPLLLLLPLLWAG | P20138 | ATGCCTCTTCTGCTTTTGCTTCC |
| | ALA (SEQ ID | | TCTTTTGTGGGCAGGTGCCCTCG |
| | NO: 273) | | CA (SEQ ID NO: 274) |
| IL-6 | MNSFSTSAFGPVA | P05231 | ATGAACTCTTTCTCAACCTCTGC |
| | FSLGLLLVLPAAF | | GTTTGGTCCGGTCGCTTTCTCCC |
| | PAP (SEQ ID | | TTGGGCTCCTGCTTGTCTTGCCA |
| | NO: 275) | | GCAGCGTTTCCTGCGCCA (SEQ |
| | | | ID NO: 276) |
| IL-8 | MTSKLAVALLAAF | P10145 | ATGACAAGTAAACTGGCGGTAGC |
| | LISAALC (SEQ | | CTTGCTCGCGGCCTTTTTGATTT |
| | ID NO: 277) | | CCGCAGCCCTTTGT (SEQ ID |
| | | | NO: 278) |
| CCL2 | MKVSAALLCLLLI | P13500 | ATGAAGGTAAGTGCAGCGTTGCT |
| | AATFIPQGLA | | TTGCCTTCTCCTCATTGCAGCGA |
| | (SEQ ID NO: | | CCTTTATTCCTCAAGGGCTGGCC |
| | 279) | | (SEQ ID NO: 280) |
| TIMP2 | MGAAARTLRLALG | P16035 | ATGGGAGCGGCAGCTAGAACACT |
| | LLLLATLLRPADA | | TCGACTTGCCCTTGGGCTCTTGC |
| | (SEQ ID NO: | | TCCTTGCAACCCTCCTTAGACCT |
| | 281) | | GCCGACGCA (SEQ ID NO: |
| | | | 282) |
| VEGFB | MSPLLRRLLLAAL | P49765 | ATGTCACCGTTGTTGCGGAGATT |
| | LQLAPAQA (SEQ | | GCTGTTGGCCGCACTTTTGCAAC |
| | ID NO: 283) | | TGGCTCCTGCTCAAGCC (SEQ |
| | | | ID NO: 284) |
| Osteoprotegerin | MNNLLCCALVFLD | O00300 | ATGAATAACCTGCTCTGTTGTGC |
| | ISIKWTTQ (SEQ | | GCTCGTGTTCCTGGACATTTCTA |
| | ID NO: 285) | | TAAAATGGACAACGCAA (SEQ |
| | | | ID NO: 286) |
| Serpin E1 | MQMSPALTCLVLG | P05121 | ATGCAAATGTCTCCTGCCCTTAC |
| | LALVFGEGSA | | CTGTCTCGTACTTGGTCTTGCGC |
| | (SEQ ID NO: | | TCGTATTTGGAGAGGGATCAGCC |
| | 287) | | (SEQ ID NO: 288) |
| GROalpha | MARAALSAAPSNP | P09341 | ATGGCAAGGGCTGCACTCAGTGC |
| | RLLRVALLLLLLV | | TGCCCCGTCTAATCCCAGATTGC |
| | AAGRRAAG (SEQ | | TTCGAGTTGCATTGCTTCTTCTG |
| | ID NO: 289) | | TTGCTGGTTGCAGCTGGTAGGAG |
| | | | AGCAGCGGGT (SEQ ID NO: |
| | | | 290) |
| CXCL12 | MNAKVVVVLVLVL | P48061 | ATGAATGCAAAAGTCGTGGTCGT |
| | TALCLSDG (SEQ | | GCTGGTTTTGGTTCTGACGGCGT |
| | ID NO: 291) | | TGTGTCTTAGTGATGGG (SEQ |
| | | | ID NO: 292) |
| IL-21 (Codon | MERIVICLMVIFL | Q9HBE4 | ATGGAACGCATTGTGATCTGCCT |
| Optimized) | GTLVHKSSS | | GATGGTCATCTTCCTGGGCACCT |
| | (SEQ ID NO: | | TAGTGCACAAGTCGAGCAGC |
| | 293) | | (SEQ ID NO: 294) |
| CD8a | MALPVTALLLPLA | P01732 | ATGGCTCTCCCTGTAACTGCCCT |
| | LLLHAARP | | GCTTCTTCCCCTTGCCTTGCTTC |
| | (SEQ ID NO: | | TCCATGCCGCTAGACCC (SEQ |
| | 295) | | ID NO: 296) |
| GMCSFR | MLLLVTSLLLCEL | P15509 | ATGCTGCTGCTGGTCACATCTCT |
| | PHPAFLLIP | | GCTGCTGTGCGAGCTGCCCCATC |
| | (SEQ ID NO: | | CTGCCTTTCTGCTGATCCCT |
| | 297) | | (SEQ ID NO: 298) |
| | | | ATGCTGCTGCTGGTTACATCTCT |
| | | | GCTGCTGTGCGAGCTGCCCCATC |
| | | | CTGCCTTTCTGCTGATCCCT |
| | | | (SEQ ID NO: 299) |

The present disclosure also encompasses additivity and synergy between an immunomodulating effector molecule(s) and the engineered cell from which they are produced. In some embodiments, cells are engineered to produce one or more (e.g., 1, 2, 3, 4, 5, 6, 7, 8, 9, 10, 11, 12, 13, 14, 15, 16, 17, 18, 19, 20 or more) immunomodulating effector molecules, each of which may modulate a different tumor-mediated immunosuppressive mechanism. In other embodiments, cells are engineered to produce at least one immunomodulating effector molecule that is not natively produced by the cells. Such an immunomodulating effector molecule may, for example, complement the function of immunomodulating effector molecules natively produced by the cells.

In some embodiments, engineered nucleic acids are configured to produce multiple inmmunomodulating effector molecules in addition to the GPC3 CARs of the present disclosure, as described further below. For example, nucleic acids may be configured to produce 2-20 different immunomodulating effector molecules. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 immunomodulating effector molecules. In some embodiments, nucleic acids are configured to produce 1, 2, 3, 4, 5, 6, 7, 8, 9, 10, 11, 12, 13, 14, 15, 16, 17, 18, 19, or 20 immunomodulating effector molecules.

In some embodiments, expression of the one or more immunomodulating effectors is controlled by an activation-conditional control polypeptide (ACP).

In some embodiments, the engineered cells further include an expression cassette comprising a promoter operably linked to an exogenous polynucleotide sequence encoding an activation-conditional control polypeptide (ACP); and an expression cassette including an ACP-responsive promoter operably linked to an exogenous polynucleotide sequence encoding at least one immunomodulating effector and having the formula: (L-E)_{X} wherein E comprises a polynucleotide sequence encoding an immunomodulating effector molecule, L comprises a linker polynucleotide sequence, X=1 to 20, wherein for the first iteration of the (L-E) unit, L is absent, and wherein the ACP is capable of inducing expression of the expression cassette encoding the at least one immunomodulating effector by binding to the ACP-responsive promoter. In some embodiments, X can be 1, 2, 3, 4, 5, 6, 7, 8, 9, 10, 11, 12, 13, 14, 15, 16, 17, 18, 19, 20, or more.

In some embodiments, the engineered cells of the present disclosure comprise two engineered nucleic acids, a first engineered nucleic acid comprising a polynucleotide sequence encoding the GPC3 CAR, and a second engineered nucleic acid comprising a polynucleotide sequence encoding an ACP. In some embodiments, the engineered cells of the present disclosure comprise three engineered nucleic acids, a first engineered nucleic acid comprising a polynucleotide sequence encoding the GPC3 CAR, and a second engineered nucleic acid comprising a polynucleotide sequence encoding an ACP, and a third engineered nucleic acid comprising a polynucleotide sequence encoding an immunomodulating effector molecule.

In some embodiments, polynucleotides encoding a GPC3 CAR, an ACP, and/or an immunomodulating effector molecule are encoded by a single polynucleotide sequence in the engineered cells. For example, in some embodiments, the engineered cell comprises a single engineered nucleic acid comprising a polynucleotide sequence encoding both the GPC3 CAR and the ACP. Other illustrative examples include, but are not limited to, (1) a chimeric antigen receptor expression cassette and an immunomodulating effector molecule expression cassette can be encoded by a first engineered nucleic acid, and an ACP expression cassette can be encoded by a second engineered nucleic acid; (2) an ACP expression cassette and an immunomodulating effector molecule expression cassette can be encoded by a first engineered nucleic acid, and a chimeric antigen receptor expression cassette can be encoded by a second engineered nucleic acid; (3) an ACP expression cassette and a chimeric antigen receptor expression cassette can be encoded by a first engineered nucleic acid, and an immunomodulating effector molecule expression cassette can be encoded by a second engineered nucleic acid.

In some embodiments, expression cassettes of polynucleotide sequences in engineered cells can be multicistronic, i.e., more than one separate polypeptide (e.g., multiple exogenous polynucleotides or immunomodulating effector molecules) can be produced from a single mRNA transcript. For example, a multicistronic expression cassette can encode both an ACP and chimeric antigen receptor, e.g., both expressed from a single expression cassette driven by a constitutive promoter. In another example, a multicistronic expression cassette can encode both an immunomodulating effector molecule and a chimeric antigen receptor, e.g., both expressed from a single expression cassette driven by an ACP-responsive promoter. Expression cassettes can be multicistronic through the use of various linkers, e.g., a polynucleotide sequence encoding a first protein of interest can be linked to a nucleotide sequence encoding a second protein of interest, such as in a first gene:linker:second gene 5′ to 3′ orientation. Multicistronic features and options are described in the section “Multicistronic and Multiple Promoter Systems.”

In some embodiments, the second expression cassette comprises two or more units of (L-E)ₓ, each L linker polynucleotide sequence is operably associated with the translation of each immunomodulating effector molecule as a separate polypeptide. In some embodiments, the second expression cassette comprising one or more units of (L-E)ₓ further comprises a polynucleotide sequence encoding a secretion signal peptide. In some embodiments, for each X the corresponding secretion signal peptide is operably associated with the immunomodulating effector molecule. In some embodiments, each secretion signal peptide comprises a native secretion signal peptide native to the corresponding immunomodulating effector molecule. In some embodiments, each secretion signal peptide comprises a non-native secretion signal peptide that is non-native to the corresponding immunomodulating effector molecule. In some embodiments, the non-native secretion signal peptide is selected from: IL12, IL2, optimized IL2, trypsiongen-2, Gaussia luciferase, CD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, when the second expression cassette comprises two or more units of (L₁-E)ₓ, each L₁ linker polynucleotide sequence is operably associated with the translation of each immunomodulating effector molecule as a separate polypeptide.

In some embodiments, the cells are engineered to include an additional expression cassette comprising an additional promoter operably linked to an additional exogenous nucleotide sequence encoding an additional immunomodulating effector molecule, for example, one that stimulates an immune response. In some embodiments, the engineered cell further comprises an additional expression cassette comprising an additional promoter and an additional exogenous polynucleotide sequence having the formula: (L-E)ₓ wherein E comprises a polynucleotide sequence encoding an immunomodulating effector molecule, L comprises a linker polynucleotide sequence, X=1 to 20, wherein the additional promoter is operably linked to the additional exogenous polynucleotide, and wherein for the first iteration of the (L-E) unit, L is absent.

In some embodiments, the additional expression cassette comprises two or more units of (L-E)ₓ, each L linker polynucleotide sequence is operably associated with the translation of each immunomodulating effector molecule as a separate polypeptide. In some embodiments, the additional expression cassette comprises one or more units of (L-E)ₓ further comprises a polynucleotide sequence encoding a secretion signal peptide. In some embodiments, for each X the corresponding secretion signal peptide is operably associated with the immunomodulating effector molecule. In some embodiments, each secretion signal peptide comprises a native secretion signal peptide native to the corresponding immunomodulating effector molecule. In some embodiments, each secretion signal peptide comprises a non-native secretion signal peptide that is non-native to the corresponding immunomodulating effector molecule. In some embodiments, the non-native secretion signal peptide is selected from IL12, IL2, optimized IL2, trypsiongen-2, Gaussia luciferase, CD5, CD8, human IgKVIICD5, CD8, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, GM-CSFR, GM-CSF, and CXCL12.

In some embodiments, engineered cells comprise one or more engineered nucleic acids comprising an expression cassette comprising a promoter and an exogenous polynucleotide sequence encoding an activation-conditional control polypeptide (ACP), wherein the promoter is operably linked to the exogenous polynucleotide and an additional expression cassette comprising an ACP-responsive promoter and an additional exogenous polynucleotide sequence having the formula: (L-E)ₓ wherein E comprises a polynucleotide sequence encoding an immunomodulating effector molecule, L comprises a linker polynucleotide sequence, X=1 to 20 wherein the ACP-responsive promoter is operably linked to the second exogenous polynucleotide, wherein for the first iteration of the (L-E) unit, L is absent, and wherein the ACP is capable of inducing expression of the expression cassette by binding to the ACP-responsive promoter.

In some embodiments, cells are engineered to include a plurality of engineered nucleic acids, e.g., at least two engineered nucleic acids, each encoding an expression cassette comprising a promoter and an exogenous polynucleotide sequence encoding an ACP and an additional exogenous polynucleotide sequence having the formula: (L-E)ₓ wherein E comprises a polynucleotide sequence encoding an immunomodulating effector molecule, L comprises a linker polynucleotide sequence, X=1 to 20. In some embodiments, the exogenous polynucleotide sequence encodes at least one (e.g., 1, 2 or 3) immunomodulating effector molecule. The exogenous polynucleotide sequence can encode at least 1, at least 2, at least 3, at least 4, at least 5, at least 6, at least 7, at least 8, at least 9, at least 10, or more immunomodulating effector molecules. For example, cells may be engineered to comprise at least 2, at least 3, at least 4, at least 5, at least 6, at least 7, at least 8, at least 8, at least 9, at least 10, or more, engineered nucleic acids, each encoding an expression cassette comprising a promoter operably linked to an ACP polynucleotide sequence, and an additional expression cassette comprising an ACP-responsive promoter and an exogenous nucleotide sequence encoding at least one (e.g., 1, 2, 3, or more) immunomodulating effector molecules. In some embodiments, the cells are engineered to comprise 2, 3, 4, 5, 6, 7, 8, 9, 10, or more engineered nucleic acids, each encoding a first expression cassette comprising a promoter operably linked to an ACP polynucleotide sequence, and an additional expression cassette comprising an ACP-responsive promoter and an exogenous nucleotide sequence encoding at least one (e.g., 1, 2, 3, or more) immunomodulating effector molecules. In accordance with these embodiments, the cells of the present disclosure have been engineered to comprise a polynucleotide sequence encoding a GPC3 CAR, and have been further engineered to include one or both of an ACP polynucleotide sequence.

In some embodiments, engineered cells of the present disclosure comprise one or more engineered nucleic acids comprising an expression cassette comprising a promoter and an exogenous polynucleotide sequence encoding a GPC3 CAR and/or an activation-conditional control polypeptide (ACP), wherein the promoter is operably linked to the exogenous polynucleotide, and an additional expression cassette comprising an activation-conditional control polypeptide-responsive (ACP-responsive) promoter and an additional exogenous polynucleotide sequence having the formula: (L-E)ₓ wherein E comprises a polynucleotide sequence encoding an immunomodulating effector molecule, L comprises a linker polynucleotide sequence, X=1 to 20 wherein the ACP-responsive promoter is operably linked to the exogenous polynucleotide, wherein for the first iteration of the (L-E) unit, L is absent, and wherein the ACP is capable of inducing expression of the expression cassette by binding to the ACP-responsive promoter. In embodiments where the exogenous polynucleotide sequence encodes a chimeric antigen receptor (e.g., GPC3 CAR), the engineered cells may further comprise an expression cassette comprising a third promoter and a third exogenous polynucleotide sequence encoding an activation-conditional control polypeptide (ACP), wherein the third promoter is operably linked to the third exogenous polynucleotide. In some embodiments, the ACP is capable of inducing expression of the second expression cassette by binding to the ACP-responsive promoter. In some embodiments, the ACP is the chimeric antigen receptor and the ACP is capable of inducing expression of an expression cassette by binding to its cognate antigen. In some embodiments, the ACP-responsive promoter is an inducible promoter that is capable of being induced by the ACP binding to its cognate antigen.

In some embodiments, the promoter operably linked to the polynucleotide sequence encoding the GPC3 CAR and/or the promoter operably linked to the ACP is a constitutive promoter, an inducible promoter, or a synthetic promoter. In some embodiments, the first promoter and/or the additional promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, an engineered nucleic acid of the present disclosure comprises a second expression cassette comprising an ACP-responsive promoter operably linked to a second exogenous polynucleotide sequence encoding one or more immunomodulating effector molecules.

In some embodiments, an engineered nucleic acid comprises an ACP-responsive promoter operably linked to a nucleotide sequence encoding an immunomodulating effector molecule. In some embodiments, an engineered nucleic acid comprises an ACP-responsive promoter operably linked to a nucleotide sequence encoding at least 2 immunomodulating effector molecules. For example, the engineered nucleic acid may comprise an ACP-responsive promoter operably linked to a nucleotide sequence encoding at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 immunomodulating effector molecules. In some embodiments, an engineered nucleic acid comprises an ACP-responsive promoter operably linked to a nucleotide sequence encoding 1, 2, 3, 4, 5, 6, 7, 8, 9, 10, 11, 12, 13, 14, 15, 16, 17, 18, 19, 20, or more immunomodulating effector molecules.

In some embodiments, the ACP-responsive promoter comprises a minimal promoter. In some embodiments, the ACP-binding domain comprises one or more zinc finger binding sites. The ACP-binding domain can comprise 1, 2, 3, 4, 5, 6 7, 8, 9, 10, or more zinc finger binding sites. In some embodiments, the ACP-binding domain comprises one zinc finger binding site. An exemplary zinc finger binding site is shown in the sequence GGCGTAGCCGATGTCGCG (SEQ ID NO: 242). In some embodiments, the ACP-binding domain comprises more than one zinc finger binding site. Zinc finger binding sites may be separated by a DNA linker. The DNA linker may be, in some embodiments, from 5 to 40 base pairs in length. In some embodiments, the ACP-binding domain comprises two zinc finger binding sites. In some embodiments, the ACP-binding domain comprises three zinc finger binding sites. In some embodiments, the ACP-binding domain comprises four zinc finger binding sites. An exemplary ACP-binding domain including four zinc finger binding sites is shown in the sequence cgggtttcgtaacaatcgcatgaggattcgcaacgccttcGGCGTAGCCGATGTCGCGctcccgtctcagtaaaggtc GGCGTAGCCGATGTCGCGcaatcggactgccttcgtacGGCGTAGCCGATGTCGCGcgtatcagtcg cctcggaacGGCGTAGCCGATGTCGCG (SEQ ID NO: 243). An exemplary ACP-responsive promoter having an ACP-binding domain that includes four zinc finger binding sites is shown in the sequence

(SEQ ID NO: 244)
cgggtttcgtaacaatcgcatgaggattcgcaacgccttcGGCGTAGCCG
ATGTCGCGctcccgtctcagtaaaggtcGGCGTAGCCGATGTCGCGcaat
cggactgccttcgtacGGCGTAGCCGATGTCGCGcgtatcagtcgcctcg
gaacGGCGTAGCCGATGTCGCGcattcgtaagaggctcactctcccttac
acggagtggataACTAGTTCTAGAGGGTATATAATGGGGGCCA.

In some embodiments, the ACP-responsive promoter comprises an enhancer that promotes transcription when a chimeric antigen receptor engages a cognate antigen, e.g., an antigen expressed on a target cell. Illustrative non-limiting examples of genes from which enhancers can be derived include, but are not limited to, ATF2, ATF7, BACH1, BATF, Bcl-6, Blimp-1, BMI1, CBFB, CREB1, CREM, CTCF, E2F1, EBF1, EGR1, ETV6, FOS, FOXA1, FOXA2, GATA3, HIF1A, IKZF1, IKZF2, IRF4, JUN, JUNB, JUND, Lef1, NFAT, NFIA, NFIB, NFKB, NR2F1, Nur77, PU.1, RELA, RUNX3, SCRT1, SCRT2, SP1, STAT4, STAT5A, T-Bet, Tcf7, ZBED1, ZNF143, or ZNF217.

In some embodiments, the ACP-responsive promoter comprises a promoter that promotes transcription when a receptor engages a cognate ligand, such as in an activation inducible system. In some embodiments, the ACP-responsive promoter comprises a promoter that promotes transcription when a chimeric antigen receptor engages a cognate antigen, e.g., an antigen expressed on a target cell. For example, when the ACP is an antigen receptor (e.g., a CAR), the ACP-responsive promoter can include promoters that are induced by signal transduction following antigen receptor binding to a cognate antigen. ACP-responsive promoters can include promoters with increased transcriptional activity in activated T cells and/or NK cells. ACP-responsive promoters can include promoters derived from genes that are upregulated in activated cells, such as T cells and/or NK cells. ACP-responsive promoters can include promoters derived from genes that have increased transcription factor binding in activated cells, such T cells and/or NK cells. Derived promoters can include the genomic region 2 kb upstream of the gene. Derived promoters can include the genomic region −100 bp downstream of the transcription initiation site the gene. Derived promoters can include the genomic region 2 kb upstream of the gene to −100 bp downstream of the transcription initiation site the gene. Derived promoters can include the genomic region upstream of the translation initiation site the gene. Derived promoters can include the genomic region 2 kb upstream to the translation initiation site the gene. Derived promoters can include one or more enhancers identified in a promoter region. ACP-responsive promoters can include, but are not limited to, promoters derived from CCL3, CCL4, or MTA2 genes. ACP-responsive promoters can include, but are not limited to, a CCL3 promoter region, a CCL4 promoter region, and/or a MTA2 promoter region. ACP-responsive promoters can include enhancers present in a CCL3 promoter region, a CCL4 promoter region, and/or a MTA2 promoter region. ACP-responsive promoters can include synthetic promoters. For example, ACP-responsive promoters can include antigen induced enhancers or promoter sequences combined with other promoters, such as minimal promoters (e.g., min AdeP or YB-TATA). ACP-responsive promoters can include synthetic enhancers, such as promoters including multiple iterations of transcription factor binding sites. In an illustrative non-limiting example, ACP-responsive promoter including a synthetic promoter can include 5 iterations of NFAT transcription factor binding sites in combination with a minimal Ade promoter (5×NFAT_minAdeP).

In some embodiments, the first expression cassette and the second expression cassette are encoded by separate polynucleotide sequences. In some embodiments, a first expression cassette and a second expression cassette are encoded by a single polynucleotide sequence.

In one aspect, provided herein are engineered cells comprising exogenous nucleic acids comprising a first expression cassette comprising a first promoter and a first exogenous polynucleotide sequence encoding a chimeric antigen receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)ₓ wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent. Expression of the second expression cassette can be induced by an ACP binding to the ACP-responsive promoter. An ACP can be a receptor, such as a chimeric antigen receptor (CAR) of the present disclosure, can induce expression of the second expression cassette upon ACP binding to a cognate ligand (e.g., a cognate antigen), such as downstream signaling following ligand binding inducing expression from an ACP-responsive promoter. In a non-limiting illustrative example, an ACP can be a GPC3 CAR, and upon CAR binding to a cognate antigen (e.g., GPC3), downstream signaling (e.g., T cell or NK cell receptor signaling) can induce expression of a cytokine payload (e.g., cytokine armoring) from an ACP-responsive promoter that is specific to CAR binding of a target antigen. Examples of ACP-responsive promoters useful for in activation inducible systems are described below. In some embodiments, the CAR induces expression of a cytokine payload that facilitates activation of NK cells and/or CD8+ cytotoxic T lymphocytes.

In some embodiments, a single engineered nucleic acid comprises at least one, two, three four, five, or more expression cassettes. In general, each expression cassette refers to a promoter operably linked to a polynucleotide sequence encoding protein of interest. For example, each of an ACP, an effector molecule, and a chimeric antigen receptor can be encoded by a separate expression cassette on the same engineered nucleic acid (e.g., vector). The expression cassettes can be oriented in any direction relative to each other (e.g., the cassettes can be in the same orientation or the opposite orientation). In exemplary engineered nucleic acids with three or more expression cassettes the cassettes can be in the same orientation or a mixed orientation.

In some embodiments, one or more engineered nucleic acids can comprise at least one, two, three four, five, or more expression cassettes. In one aspect, engineered expression systems are provided herein that include (a) a first expression cassette comprising a first promoter and a first exogenous polynucleotide sequence encoding a chimeric antigen receptor, wherein the first promoter is operably linked to the first exogenous polynucleotide; and (2) a second expression cassette comprising an ACP-responsive promoter and a second exogenous polynucleotide sequence having the formula: (L-E)ₓ wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and wherein the ACP is capable of inducing expression of the second expression cassette by binding to the ACP-responsive promoter. The first expression cassette and the second expression cassette may be encoded by separate polynucleotide sequences, or alternatively may be encoded by the same polynucleotide sequence.

As illustrative non-limiting examples of expression systems, (1) a chimeric antigen receptor expression cassette and an immunomodulating effector molecule expression cassette can be encoded by a first engineered nucleic acid, and an ACP expression cassette can be encoded by a second engineered nucleic acid; (2) an ACP expression cassette and an immunomodulating effector molecule expression cassette can be encoded by a first engineered nucleic acid, and a chimeric antigen receptor expression cassette can be encoded by a second engineered nucleic acid; (3) an ACP expression cassette and a chimeric antigen receptor expression cassette can be encoded by a first engineered nucleic acid, and an effector molecule expression cassette can be encoded by a second engineered nucleic acid. In an additional illustrative non-limiting example, an immunomodulating effector molecule expression cassette can be encoded by a first engineered nucleic acid, and an ACP expression cassette can be encoded by a second engineered nucleic acid.

In some embodiments, expression cassettes can be multicistronic. For example, a multicistronic expression cassette can encode both an ACP and chimeric antigen receptor, e.g., both expressed from a single expression cassette driven by a constitutive promoter. In another example, a multicistronic expression cassette can encode both an immunomodulating effector molecule and a chimeric antigen receptor, e.g., both expressed from a single expression cassette driven by an ACP-responsive promoter.

In some embodiments, the engineered nucleic acid is selected from: a DNA, a cDNA, an RNA, an mRNA, and a naked plasmid. Also provided herein is an expression vector comprising the engineered nucleic acid.

In some embodiments, the engineered cells of the present disclosure include a nucleic acid that further comprises an insulator. The insulator can be localized between the first expression cassette and the second expression cassette. An insulator is a cis-regulatory element that has enhancer-blocking or barrier function. Enhancer-blocker insulators block enhancers from acting on the promoter of nearby genes. Barrier insulators prevent euchromatin silencing. Examples of suitable insulators include, without limitation, an A1 insulator, a CTCF insulator, a gypsy insulator, an HS5 insulator, and a β-globin locus insulator, such as cHS4. In some embodiments, the insulator is an A2 insulator, an A1 insulator, a CTCF insulator, an HS5 insulator, a gypsy insulator, a β-globin locus insulator, or a cHS4 insulator. In some embodiments, the insulator may be an A2 insulator.

In some embodiments, the ACP may be a transcriptional modulator. In some embodiments, the ACP is a transcriptional repressor. In some embodiments, the ACP is a transcriptional activator. In some embodiments, the ACP is a transcription factor. In some embodiments, the ACP comprises a DNA-binding domain and a transcriptional effector domain. In some embodiments, the transcription factor is a zinc-finger-containing transcription factor. In some embodiments, the zinc-finger-containing transcription factor may be a synthetic transcription factor. In some embodiments, the ACP DNA-binding domain comprises a DNA-binding zinc finger protein domain (ZF protein domain) and an effector domain. In some embodiments, the DNA-binding domain comprises a tetracycline (or derivative thereof) repressor (TetR) domain.

In some embodiments, the DNA-binding domain is a ZF protein domain. In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

In some embodiments, the ZF protein domain includes an array of six zing finger motifs. An exemplary ZF protein domain including an array of six zinc finger motifs is shown in the sequence

(SEQ ID NO: 191)
SRPGERPFQCRICMRNFSRRHGLDRHTRTHTGEKPFQCRICMRNFSDHSS
LKRHLRTHTGSQKPFQCRICMRNFSVRHNLTRHLRTHTGEKPFQCRICMR
NFSDHSNLSRHLKTHTGSQKPFQCRICMRNFSQRSSLVRHLRTHTGEKPF
QCRICMRNFSESGHLKRHLRTHLRGS.

The ACP can also further comprise an effector domain, such as a transcriptional effector domain. For instance, a transcriptional effector domain can be the effector or activator domain of a transcription factor. Transcription factor activation domains are also known as transactivation domains, and act as scaffold domains for proteins such as transcription coregulators that act to activate or repress transcription of genes. Any suitable transcriptional effector domain can be used in the ACP including, but not limited to, a Herpes Simplex Virus Protein 16 (VP16) activation domain; an activation domain that includes, e.g., four tandem copies of VP16; a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a truncated Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, or any combination thereof.

Exemplary transcription effector domain protein sequences are shown in Table 8. Exemplary transcription effector domain nucleotide sequences are shown in Table 9.

TABLE 8
Transcriptional Effector Domain (Protein)

| | SEQ | |
|---|---|---|
| | ID | |
| Amino Acid Sequence | NO: | Description |
| RTLVTFKDVFVDFTREEWKLLDTAQQIVYRNV | 192 | KRAB |
| MLENYKNLVSLGYQLTKPDVILRLEKGEEPWL | | |
| V | | |
| RTLVTFKDVFVDFTREEWKLLDTAQQIVYRNV | 193 | truncated |
| MLENYKNLVSLGY | | KRAB |
| | | (min KRAB) |
| EASGSGRADALDDFDLDMLGSDALDDFDLDML | 194 | VPR |
| GSDALDDFDLDMLGSDALDDFDLDMLINSRSS | | activation |
| GSPKKKRKVGSQYLPDTDDRHRIEEKRKRTYE | | domain |
| TFKSIMKKSPFSGPTDPRPPPRRIAVPSRSSA | | |
| SVPKPAPQPYPFTSSLSTINYDEFPTMVFPSG | | |
| QISQASALAPAPPQVLPQAPAPAPAPAMVSAL | | |
| AQAPAPVPVLAPGPPQAVAPPAPKPTQAGEGT | | |
| LSEALLQLQFDDEDLGALLGNSTDPAVFTDLA | | |
| SVDNSEFQQLLNQGIPVAPHTTEPMLMEYPEA | | |
| ITRLVTGAQRPPDPAPAPLGAPGLPNGLLSGD | | |
| EDFSSIADMDFSALLGSGSGSRDSREGMFLPK | | |
| PEAGSAISDVFEGREVCQPKRIRPFHPPGSPW | | |
| ANRPLPASLAPTPTGPVHEPVGSLTPAPVPQP | | |
| LDPAPAVTPEASHLLEDPDEETSQAVKALREM | | |
| ADTVIPQKEEAAICGQMDLSHPPPRGHLDELT | | |
| TTLESMTEDLNLDSPLTPELNEILDTFLNDEC | | |
| LLHAMHISTGLSIFDTSLF | | |

TABLE 9
Transcriptional Effector Domain (Nucleotide)

| | SEQ | |
|---|---|---|
| | ID | |
| Nucleic Acid Sequence | NO: | Description |
| AGAACCCTGGTCACCTTCAAGGACGTGTTCG | 195 | KRAB |
| TGGACTTCACCCGGGAAGAGTGGAAGCTGCT | | |
| GGATACAGCCCAGCAGATCGTGTACCGGAAC | | |
| GTGATGCTGGAAAACTACAAGAATCTGGTGT | | |
| CCCTGGGCTACCAGCTGACCAAGCCTGACGT | | |
| GATCCTGCGGCTGGAAAAGGGCGAAGAACCT | | |
| TGGCTGGTG | | |

| | | |
|---|---|---|
| AGAACCCTGGTCACCTTCAAGGACGTGTTCG | 196 | truncated |
| TGGACTTCACCCGGGAAGAGTGGAAGCTGCT | | KRAB |
| GGATACAGCCCAGCAGATCGTGTACCGGAAC | | (min KRAB) |
| GTGATGCTGGAAAACTACAAGAATCTGGTGT | | |
| CCCTGGGCTAC | | |

In some embodiments, the ACP is a small molecule (e.g., drug) inducible polypeptide. In some embodiments, the ACP may be induced by tamnoxifen, or a metabolite thereof, such as 4-hydroxy-tainoxifen (4-OHT), and comprises an estrogen receptor variant, such as ERT2. In some embodiments, the ACP is a small molecule (e.g., drug) inducible polypeptide that comprises a repressible protease and one or more cognate cleavage sites of the repressible protease.

The term “repressible protease” as used herein, refers to a protease that can be inactivated by the presence or absence of a specific agent (e.g., that binds to the protease). In some embodiments, a repressible protease is active (cleaves a cognate cleavage site) in the absence of the specific agent and is inactive (does not cleave a cognate cleavage site) in the presence of the specific agent. In some embodiments, the specific agent is a protease inhibitor.

Non-limiting examples of repressible proteases include hepatitis C virus proteases (e.g., NS3 and NS2-3); signal peptidase; proprotein convertases of the subtilisin/kexin family III (furin, PCI, PC2, PC4, PACE4, PC5, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N; insulin degrading enzyme; calpain; high molecular weight protease; and, caspases 1, 2, 3, 4, 5, 6, 7, 8, and 9.

The term “cognate cleavage site” as used herein, refers to a specific sequence or sequence motif recognized by and cleaved by the repressible protease.

Other proteases, including those listed above and in Table 10, can be used. When a protease is selected, its cognate cleavage site and protease inhibitors known in the art to bind and inhibit the protease can be used in a combination. Exemplary combinations for the use are provided below in Table 10. Representative sequences of the proteases are available from public database including UniProt through the uniprot.org website. UniProt accession numbers for the proteases are also provided below in Table 10.

TABLE 10

| Protease | | |
|---|---|---|
| (UniProt | Cognate | |
| Accession | cleavage | |
| Number) | site | Protease inhibitors |
| HCV | DEMEECSQHL | Simeprevir, Danoprevir, |
| NS4A/4B | (SEQ ID NO: | Asunaprevir, Ciluprevir, |
| | 197) | Boceprevir, Sovaprevir, |
| | EDVVPCSMG | Paritaprevir, Telaprevir, |
| | (SEQ ID NO: | Grazoprevir |
| | 198) | |
| HCV | DEMEECSQHL | Simeprevir, Danoprevir, |
| NS5A/5B | (SEQ ID NO: | Asunaprevir, Ciluprevir, |
| | 199) | Boceprevir, Sovaprevir, |
| | EDVVPCSMG | Paritaprevir, Telaprevir, |
| | (SEQ ID NO: | Grazoprevir |
| | 200) | |
| HCV NS3 | DEMEECSQHL | Simeprevir, Danoprevir, |
| | (SEQ ID NO: | Asunaprevir, Ciluprevir, |
| | 201) | Boceprevir, Sovaprevir, |
| | EDVVPCSMG | Paritaprevir, Telaprevir, |
| | (SEQ ID NO: | Grazoprevir |
| | 202) | |
| HCV NS2-3 | DEMEECSQHL | Simeprevir, Danoprevir, |
| | (SEQ ID NO: | Asunaprevir, Ciluprevir, |
| | 203) | Boceprevir, Sovaprevir, |
| | EDVVPCSMG | Paritaprevir, Telaprevir, |
| | (SEQ ID NO: | Grazoprevir |
| | 204) | |
| NS3/NS4 | EDVVCCHSIY | Simeprevir, Danoprevir, |
| protease | (SEQ ID NO: | Asunaprevir, Ciluprevir, |
| cleavage | 205) | Boceprevir, Sovaprevir, |
| site | LYQEFDEMEEC | Paritaprevir, Telaprevir, |
| | SQH (SEQ ID | Grazoprevir |
| | NO: 206) | |

In some embodiments, the one or more cognate cleavage sites of the repressible protease are localized between the DNA-binding domain and the effector domain of the ACP. In some embodiments, the repressible protease is hepatitis C virus (HCV) nonstructural protein 3 (NS3). In some embodiments, the cognate cleavage site comprises an NS3 protease cleavage site. In some embodiments, the NS3 protease cleavage site comprises a NS3/NS4A, a NS4A/NS4B, a NS4B/NS5A, or a NS5A/NS5B junction cleavage site.

In some embodiments, the NS3 protease can be repressed by a protease inhibitor. Any suitable protease inhibitor can be used, including, but not limited to, simeprevir, danoprevir, asunaprevir, ciluprevir, boceprevir, sovaprevir, paritaprevir, telaprevir, grazoprevir, glecaprevir, and voxiloprevir, or any combination thereof. In some embodiments, the protease inhibitor is selected from: simeprevir, danoprevir, asunaprevir, ciluprevir, boceprevir, sovaprevir, paritaprevir, telaprevir, grazoprevir, glecaprevir, and voxiloprevir. In some embodiments, the protease inhibitor is grazoprevir. In some embodiments, the protease inhibitor is a combination of grazoprevir and elbasvir (a NS5A inhibitor of the hepatitis C virus NS5A replication complex).

In some embodiments, an ACP of the present disclosure comprises a small molecule (e.g., drug) inducible hormone-binding domain of estrogen receptor (ERT2 domain). In some embodiments, the ERT2 domain is an estrogen receptor variant that binds to tamoxifen, and metabolites thereof, but not to estradiol. Non-limiting examples of tamoxifen metabolites may include 4-hydroxytamoxifen, N-desmethyltamoxifen, tamoxifen-N-oxide, and endoxifen. In some embodiments, when expressed in a cell and in the absence of the small molecule (e.g., tamoxifen or a metabolite thereof) the ACP comprising the ERT2 domain binds to HSP90 and is maintained in the cytoplasm of the cell. In some embodiments, upon introduction of the small molecule (e.g., tamoxifen or a metabolite thereof), the small molecule displaces HSP90 bound to the ERT2 domain, which allows the ACP comprising the ERT2 domain to translocate to the nucleus of the cell.

Accordingly, in some embodiments an ACP of the present disclosure comprising an ERT2 domain is capable of undergoing nuclear localization upon binding of the ERT2 domain to tamoxifen or a metabolite thereof. In some embodiments, the tamoxifen metabolite is selected from 4-hydroxy-tamoxifen (4-OHT), N-desmethyltamoxifen, tamoxifen-N-oxide, and endoxifen.

In some embodiments, the ACP further comprises a degron, wherein the degron is operably linked to the ACP. In some embodiments, the degron is localized 5′ of the repressible protease, 3′ of the repressible protease, 5′ of the DNA-binding domain, 3′ of the DNA-binding domain, 5′ of the effector domain, or 3′ of the effector domain.

The terms “degron” “degron domain,” as used herein, refers to a protein or a part thereof that is important in regulation of protein degradation rates. Various degrons known in the art, including but not limited to short amino acid sequences, structural motifs, and exposed amino acids, can be used in various embodiments of the present disclosure.

In some embodiments, the degron is selected from: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

In some embodiments, the degron comprises a cereblon (CRBN) polypeptide substrate domain capable of binding CRBN in response to an immunomodulatory drug (IMiD) thereby promoting ubiquitin pathway-mediated degradation of the ACP. In some embodiments, the CRBN polypeptide substrate domain is selected from: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some embodiments, the CRBN polypeptide substrate domain is a chimeric fusion product of native CRBN polypeptide sequences. In some embodiments, the CRBN polypeptide substrate domain is a IKZF3/ZFP91/IKZF3 chimeric fusion product having the amino acid sequence of

(SEQ ID NO: 207)
FNVLMVHKRSHTGERPLQCEICGFTCRQKGNLLRHIKLHTGEKPFKCHLC
NYACQRRDAL

In some embodiments, the immunomodulatory drug (IMiD) is an FDA-approved drug. In some embodiments, the IMiD is selected from: thalidomide, lenalidomide, and pomalidomide.

Engineered Cell Types

An engineered cell or isolated cell of the present disclosure can be a human cell. An engineered cell or isolated cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer cell. An engineered primary cell can be any somatic cell. An engineered primary cell can be an MSC. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered or isolated cell of the present disclosure is selected from: a T cell, a CD8+ T cell, a CD4+ T cell, a gamma-delta T cell, a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the engineered cell is a Natural Killer (NK) cell. In some embodiments, an engineered cell is autologous. In some embodiments, an engineered cell is allogeneic.

In some embodiments, an engineered cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a gastric tumor cell, a testicular yolk sac tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Methods of Engineering Cells

Also provided herein are compositions and methods for engineering cells to produce the GPC3 chimeric antigen receptors (CARs) of the present disclosure. In general, cells are engineered to produce GPC3 CARs through introduction (i.e. delivery) of one or more polynucleotides of the present disclosure comprising a promoter and an exogenous polynucleotide sequence encoding a GPC3 CAR into the cell's cytosol and/or nucleus. For example, the polynucleotide expression cassettes encoding the GPC3 CARs can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

In some embodiments, the engineered cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

The virus, including any of the oncolytic viruses described herein, can be a recombinant virus that encodes a GPC3 CAR (and, e.g., one more transgenes encoding one or more immunomodulating effector molecules), such as any of the engineered nucleic acids described herein. The virus, including any of the oncolytic viruses described herein, can be a recombinant virus that encodes a GPC3 CAR, such as any of the engineered nucleic acids described herein. In some embodiments, the cell is engineered via transduction with an oncolytic virus.

Viral-Mediated Delivery

Viral vector-based delivery platforms can be used to engineer cells. In general, a viral vector-based delivery platform engineers a cell through introducing (i.e. delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode GPC3 CARs. The one or more transgenes encoding the GPC3 CARs can be configured to express the GPC3 CARs. A viral vector-based delivery platform can encode one or more genes in addition to the one or more transgenes (e.g., transgenes encoding the GPC3 CARs), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the GPC3 CARs. One viral vector can deliver more than one engineered nucleic acids, such as one vector that delivers engineered nucleic acids that are configured to produce GPC3 CARs. More than one viral vector can deliver more than one engineered nucleic acids, such as more than one vector that delivers one or more engineered nucleic acid configured to produce GPC3 CARs. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of chimeric antigen receptors, such as GPC3 CARs, or used in vivo and ex vivo gene therapy procedures, e.g., for in vivo delivery of the engineered nucleic acids encoding GPC3 CARs. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express the GPC3 CARs. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding GPC3 CARs. The transgenes encoding the GPC3 CARs can be configured to express the GPC3 CARs.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding GPC3 CARs) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e. infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Lipid Structure Delivery Systems

Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery-A Review. Nanomaterials 2017, 7(5), 94), herein incorporated by reference for all purposes.

Genomic Editing Systems

A genomic editing systems can be used to engineer a host genome to encode an engineered nucleic acid, such as an engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding GPC3 CARs).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding the GPC3 CARs described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e. permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Iber. 2019 Apr. 10; 27(4): 710-728) and Kaczmarek et al. (Genome Med. 2017; 9: 60.), each herein incorporated by reference for all purposes.

Methods of Use

Methods for treatment of diseases are also encompassed by this disclosure. Said methods include administering a therapeutically effective amount of an engineered nucleic acid, engineered cell, or isolated cell as described above. In some aspects, provided herein are methods of treating a subject in need thereof, the method comprising administering a therapeutically effective dose of any of the engineered cells, isolated cells, or compositions disclosed herein.

In some aspects, provided herein are methods of stimulating a cell-mediated immune response to a tumor cell in a subject, the method comprising administering to a subject having a tumor a therapeutically effective dose of any of the engineered cells, isolated cells, or compositions disclosed herein.

In some aspects, provided herein are methods of providing an anti-tumor immunity in a subject, the method comprising administering to a subject in need thereof a therapeutically effective dose of any of the engineered cells, isolated cells, or compositions disclosed herein.

In some aspects, provided herein are methods of treating a subject having cancer, the method comprising administering a therapeutically effective dose of any of the engineered cells, isolated cells, or compositions disclosed herein.

In some aspects, provided herein are methods of reducing tumor volume in a subject, the method comprising administering to a subject having a tumor a composition comprising any of the engineered cells, isolated cells, or compositions disclosed herein.

In some embodiments, the administering comprises systemic administration. In some embodiments, the administering comprises intratumoral administration. In some embodiments, the isolated cell is derived from the subject. In some embodiments, the isolated cell is allogeneic with reference to the subject.

In some embodiments, the method further comprises administering a checkpoint inhibitor. the checkpoint inhibitor is selected from: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody. In some embodiments, the method further comprises administering an anti-CD40 antibody.

In some embodiments, the tumor is selected from: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

Some methods comprise selecting a subject (or patient population) having a tumor (or cancer) and treating that subject with engineered cells or delivery vehicles that modulate tumor-mediated immunosuppressive mechanisms.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells.

The methods provided herein also include administering a drug or pharmaceutical composition in combination with a therapeutically effective dose of any of the engineered cells, isolated cells, or compositions disclosed herein such that the ACP is induced and/or that a repressible protease is repressed. For example, tamoxifen or a metabolite thereof (e.g., 4-hydroxytamoxifen, N-desmethyltamoxifen, tamoxifen-N-oxide, or endoxifen) can be administered to induce the ACP. The drug or pharmaceutical can be administered prior to, concurrently with, simultaneously with, and/or subsequent to administration of any of the engineered cells, isolated cells, or compositions disclosed herein. The drug or pharmaceutical can be administered serially. The drug or pharmaceutical can be administered concurrently or simultaneously with administration of any of the engineered cells, isolated cells, or compositions disclosed herein. The drug or pharmaceutical can be administered at separate intervals than (e.g., prior to or subsequent to) administration of any of the engineered cells, isolated cells, or compositions disclosed herein. The drug or pharmaceutical can be administered both concurrently/simultaneously as well as at separate intervals than any of the engineered cells, isolated cells, or compositions disclosed herein. The drug or pharmaceutical composition and the engineered cells, isolated cells, or compositions can be administered via different routes, e.g., the drug or pharmaceutical composition can be administered orally and the engineered cells, isolated cells, or compositions can be administered intraperitoneally, intravenously, subcutaneously, or any other route appropriate for administration, as will be appreciated by one skilled in the art.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The methods provided herein include administering a protease inhibitor. In some embodiments, the NS3 protease can be repressed by a protease inhibitor. Any suitable protease inhibitor can be used, including, but not limited to, simeprevir, danoprevir, asunaprevir, ciluprevir, boceprevir, sovaprevir, paritaprevir, telaprevir, grazoprevir, glecaprevir, and voxiloprevir, or any combination thereof. In some embodiments, the protease inhibitor is selected from: simeprevir, danoprevir, asunaprevir, ciluprevir, boceprevir, sovaprevir, paritaprevir, telaprevir, grazoprevir, glecaprevir, and voxiloprevir.

In some embodiments, the protease inhibitor is grazoprevir. In some embodiments, the protease inhibitor is a combination of grazoprevir and elbasvir (a NS5A inhibitor of the hepatitis C virus NS5A replication complex). Grazoprevir and elbasvir can be co-formulated as a pharmaceutical composition, such as in tablet form (e.g., the tablet available under the tradename Zepatier®). Grazoprevir and elbasvir can be co-formulated at a 2:1 weight ratio, respectively, such as at a unit dose of 100 mg grazoprevir 50 mg elbasvir (e.g., as in the tablet available under the tradename Zepatier®). The protease inhibitor can be administered at a dose capable of repressing a repressible protease domain of an ACP. The protease inhibitor can be administered at an approved dose for another indication. As an illustrative non-limiting example, Zepatier can be administered at its approved dose for treatment of HCV.

Grazoprevir, including in combination with elbasvir, can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, grazoprevir, including in combination with elbasvir, can be provided in the form of tablets or capsules containing 1.0 to 500 mg of the active ingredient, particularly 1, 5, 10, 15, 20, 25, 50, 75, 100, 150, 200, 250, 300, 400, 500, and 750 mg of the active ingredient for the symptomatic adjustment of the dosage to the patient to be treated. Generally, a total daily dosage of grazoprevir, including in combination with elbasvir, can range from about 1 to about 2500 mg per day, although variations will necessarily occur depending on the target of therapy, the patient and the route of administration. In one embodiment, the dosage of grazoprevir, including in combination with elbasvir, is from about 10 to about 1000 mg/day, administered in a single dose or in 2-4 divided doses. In another embodiment, the dosage of grazoprevir, including in combination with elbasvir, is from about 1 to about 500 mg/day, administered in a single dose or in 2-4 divided doses. In still another embodiment, the dosage of grazoprevir, including in combination with elbasvir, is from about 1 to about 100 mg/day, administered in a single dose or in 2-4 divided doses. In yet another embodiment, the dosage of grazoprevir, including in combination with elbasvir, is from about 1 to about 50 mg/day, administered in a single dose or in 2-4 divided doses. In another embodiment, the dosage of grazoprevir, including in combination with elbasvir, is from about 500 to about 1500 mg/day, administered in a single dose or in 2-4 divided doses. In still another embodiment, the dosage of grazoprevir, including in combination with elbasvir, is from about 500 to about 1000 mg/day, administered in a single dose or in 2-4 divided doses. In yet another embodiment, the dosage of grazoprevir, including in combination with elbasvir, is from about 100 to about 500 mg/day, administered in a single dose or in 2-4 divided doses.

In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the GPC3 CARs described herein. Compositions capable of in vivo production of GPC3 CARs include, but are not limited to, any of the engineered nucleic acids described herein. Compositions capable of in vivo production of GPC3 CARs can be a naked mRNA or a naked plasmid.

Pharmaceutical Compositions

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Whether it is a polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Efficacy of GPC3 CARs in T Cells

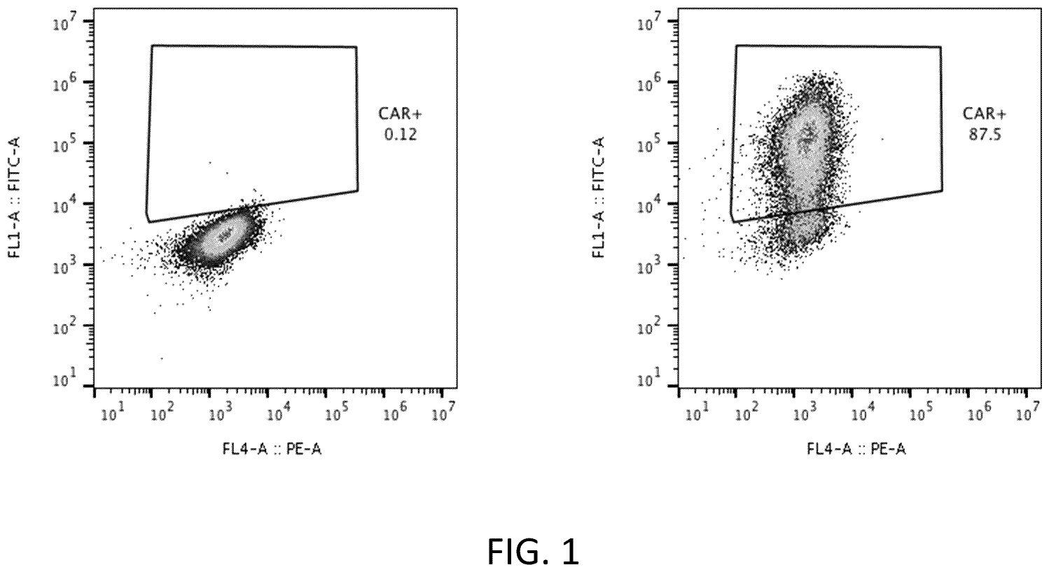

In accordance with the various embodiments described herein, experiments were conducted to evaluate killing by T cells expressing an anti-GPC3 CAR. A CAR was constructed including a GC33-derived scFV, CAR T cells were produced, and killing of tumor cells by the CAR T cells was assessed. For example, FIG. 1 includes representative data from flow cytometry experiments evaluating the expression of GC33 CARs in T cells. Briefly, cells were thawed at day 0 and pan T cells were activated. At day 1, cells were transduced with 100 k of GoStix reagent units per 1×10⁶ cells. Cell culture media was changed on day 2. On day 5, beads were removed, counted and CAR expression was checked using flow cytometry. Cells were then replated at 0.5×10⁶ cells/ml in fresh medium.

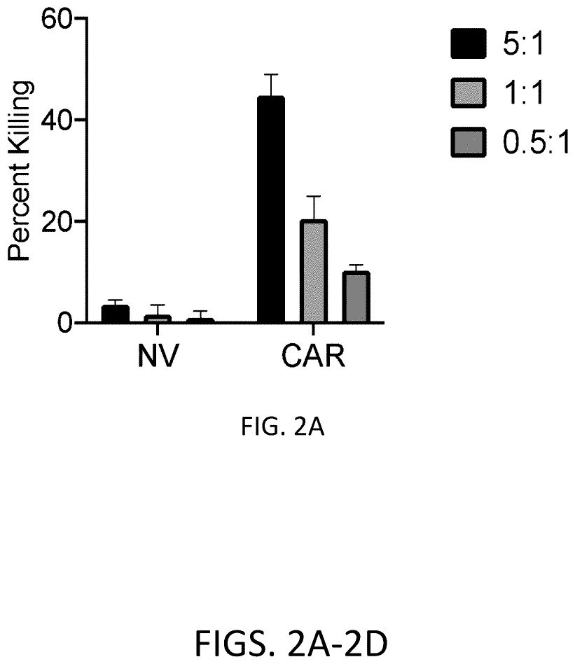

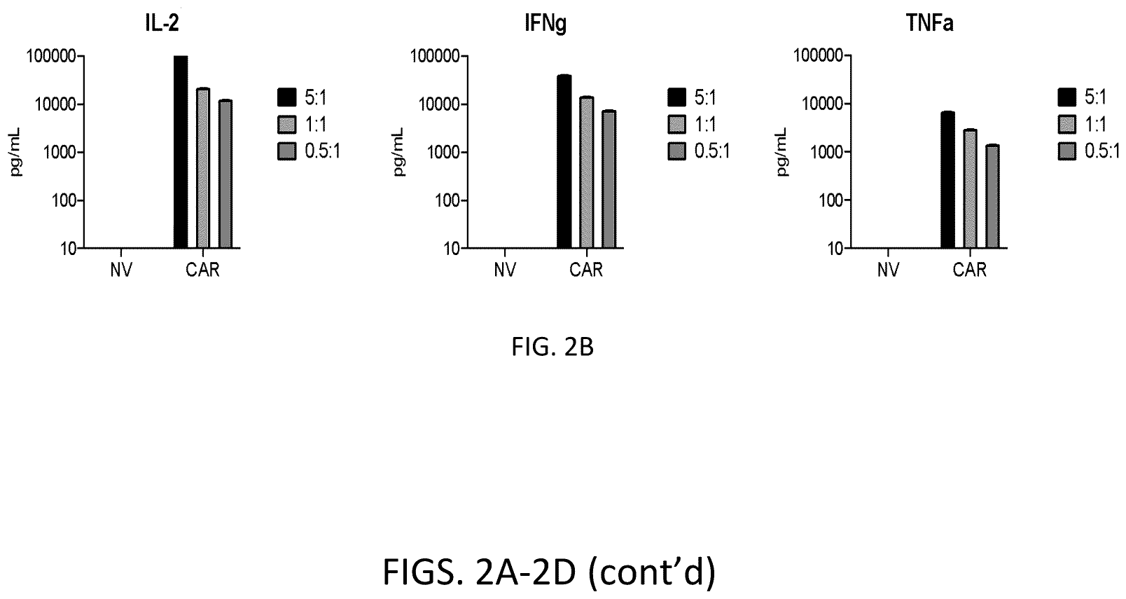

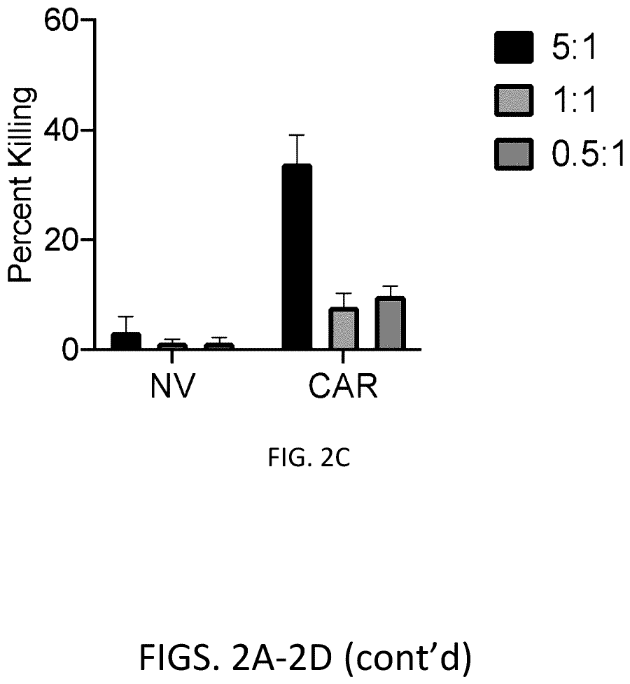

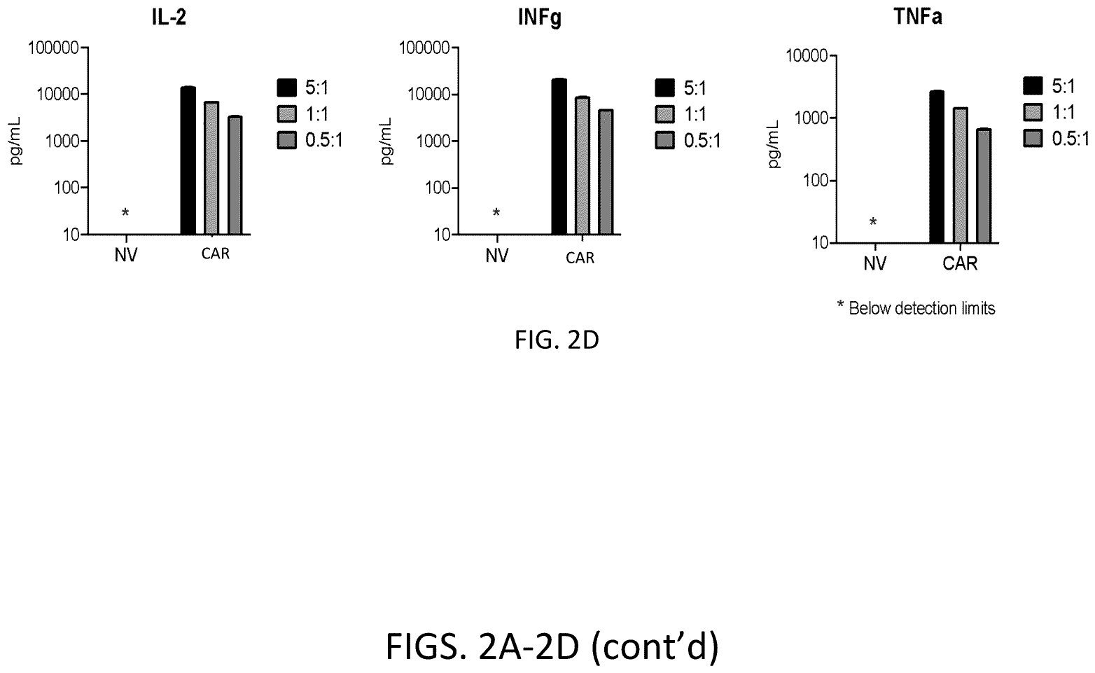

On day 8, functional assays (killing assays) were performed to test the effects of the GC33 CAR T-cells on two GPC3-expressing liver tumor cells, HepG2 and Hep3B (FIGS. 2A and 2C), and the expression of various cytokines (IL-2, INFγ, and TNFα) were evaluated in these cells (FIGS. 23 and 2D). Briefly, approximately 25,000 target cells were plated allowed to adhere at 37° C. before adding the CAR T cells (at different E:T ratios, which were normalized for Car T+ %). The cells were co-cultured in a total volume of 200 μl/well in a 96-well flat bottom plate. The killing readout was obtained at about 20 hours after co-culturing using LDH, and cytokine expression was analyzed in the supernatants at about 20 hours after co-culturing with Luminex. The results provided in FIGS. 2A-2D demonstrate effective targeting and killing of GPC3-expressing liver tumor cells (HepG2 and Hep3B) by the GC33 CAR T-cells (FIGS. 2A and 2C), and significant increases in expression of various cytokines (IL-2, INFγ, and TNFα) in these cells (FIGS. 23 and 2D).

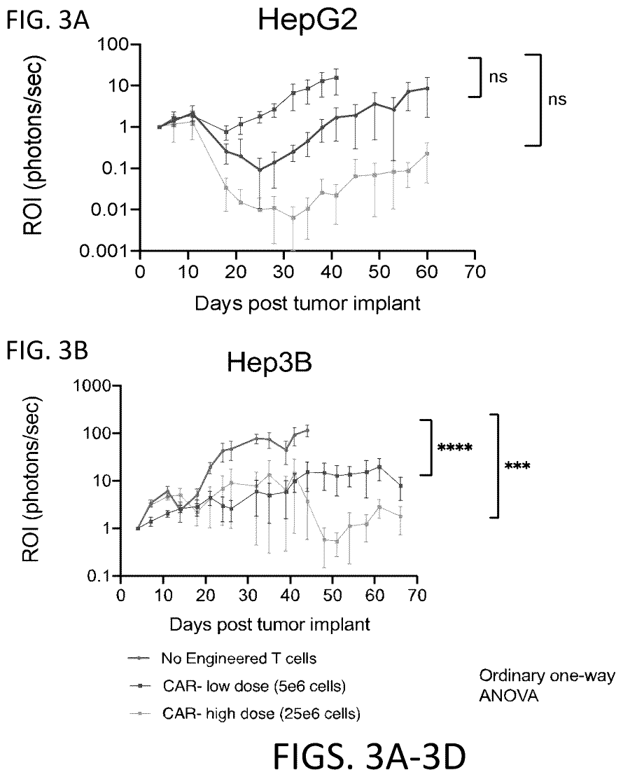

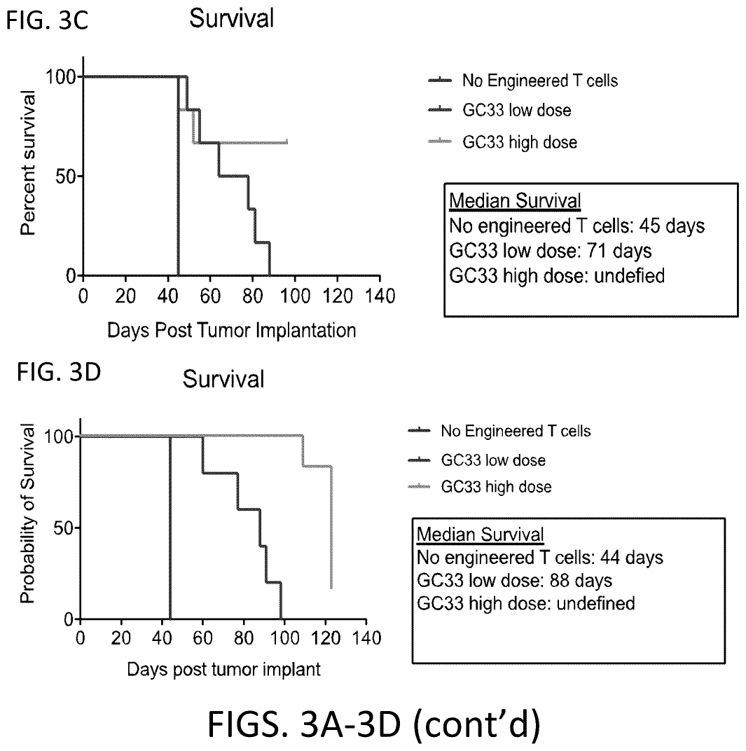

Additional experiments were conducted to evaluate the in vivo efficacy GC33 CARs against two GPC3-expressing liver tumor cells (HepG2 and Hep3B) injected into mice (FIGS. 3A-3D). Briefly, GC33 CAR dosing regimens were tested in HCC cells injected into the IP cavity of mice. About 6×10⁶ HepG2 cells and about 3×10⁶ Hep3B cells were injected. BLI and body weight measurements were obtained twice a week, and the overall health condition of the mice was assessed. Mice were sacrificed when body weight dropped more than 15% of the original weight. Tumor cells were injected at day 0, and T cells were injected at day 10. The results provided in FIGS. 3A-3D demonstrate that GPC3-specific CAR T-cells decreased the number of tumor cells present in mice post-injection with HepG2 cells (FIG. 3A), increased overall survival of HepG2-injected mice (FIG. 3C), decreased the number of tumor cells present in mice post-injection with Hep3B cells (FIG. 3B) and increased overall survival of Hep3B-injected mice (FIG. 3D).

### Example 2: Efficacy of GPC3 CARs in NK Cells

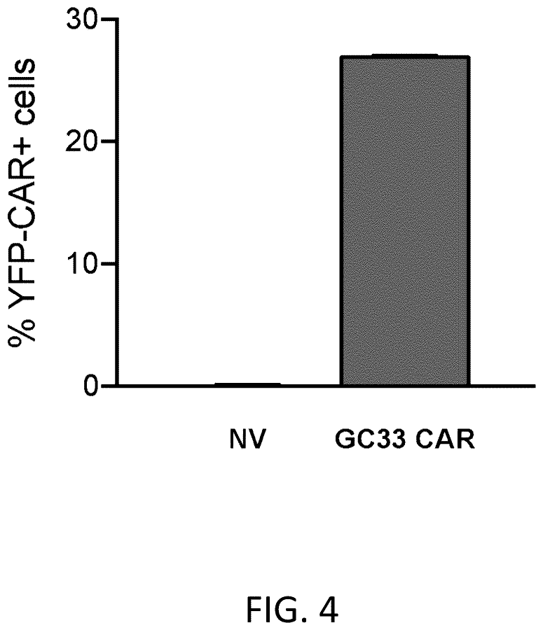

Experiments were also conducted to characterize the effects of NK cells expressing an anti-GPC3 CAR against liver tumor cells. The anti-GPC3 CAR described in Example 1 was used to transduce NK cells, and tumor cell killing by the CAR NK cells was assessed. FIG. 4 includes representative data from flow cytometry experiments demonstrating expression of GC33 CARs in NK cells. Briefly, cells were expanded to day 10 using MitoC WT K562 feeders (transduced with MOI=40 (IU titer)). Retronectin-coated plates were used, and spinoculation was performed at 800 g for 2 hrs at 32° C. The cells were incubated for 2 hrs and then transferred to 24-well Grex plates. Flow cytometry evaluation was performed on day 7 post-transduction. Cells were prepared for killing assays on day 7 and on day 10 post-transduction with two target HCC lines, HepG2 and Huh7 at a 1:1 E:T ratio. The results provided in FIG. 4 demonstrate that about 30% of NK cells expressed the GC33 CAR.

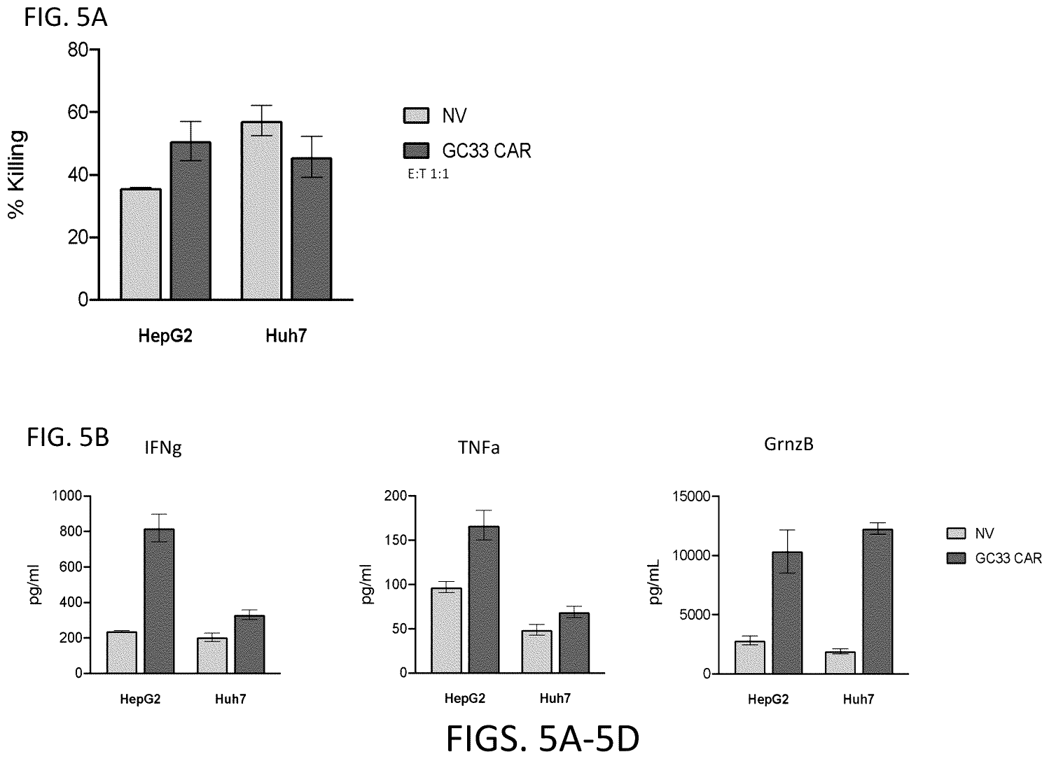

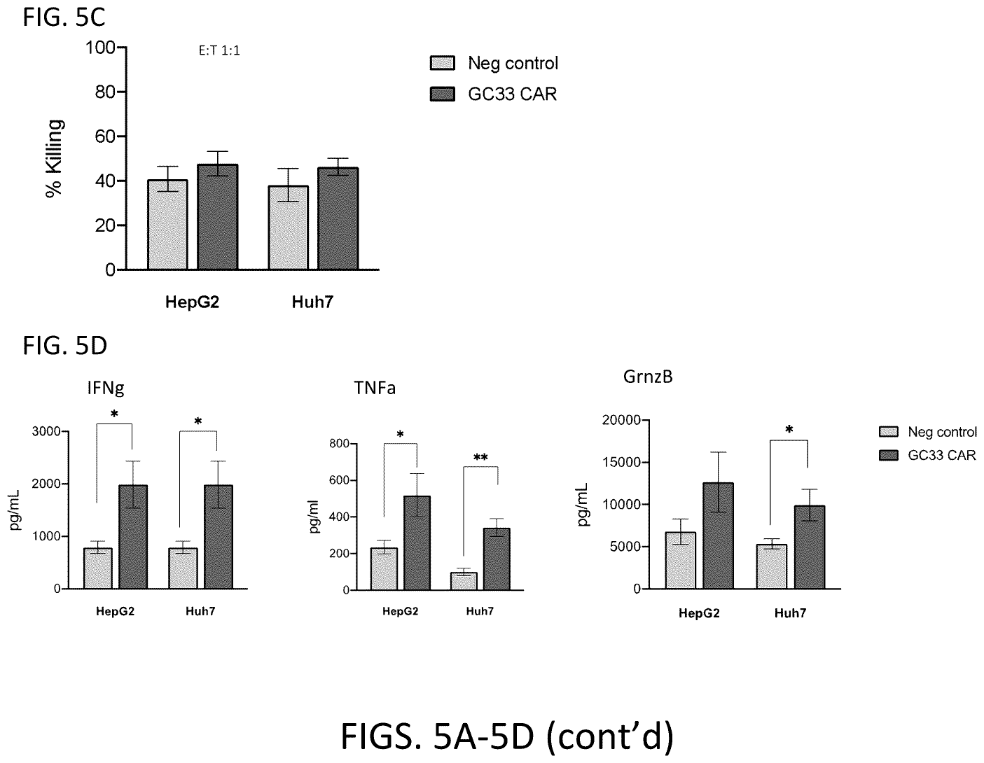

Functional assays (killing assays) were performed to test the effects of the GC33 CAR NK-cells on the two GPC3-expressing liver tumor cells seven days post-transduction (FIG. 5A) and ten days post-transduction (FIG. 5C), and the expression of various cytokines (INFγ, TNFα, GrnzB) were evaluated in these cells seven days post-transduction (FIG. 5B) and ten days post-transduction (FIG. 5D). Briefly, target cells were plated allowed to adhere at 37° C. before adding the CAR-NK cells. The cells were co-cultured in a total volume of 200 μl/well in a 96-well flat bottom plate. The killing readout was obtained at about 20 hours after co-culturing using LDH, and cytokine expression was analyzed in the supematants at about 20 hours after co-culturing with Luminex. The results provided in FIGS. 5A-5D demonstrate percent killing of GPC3-expressing liver tumor cells by the GC33 CAR NK-cells (FIGS. 5A and 5C), and significant increases in expression of various cytokines (INFγ, TNFα, GrnzB) in these cells (FIGS. 5B and 5D).

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

For reasons of completeness, various aspects of the disclosure are set out in the following numbered embodiments:

1. A chimeric antigen receptor (CAR) that binds to Glypican-3 (GPC3), wherein the CAR comprises a single chain Fv (scFv) that binds to GPC3, a transmembrane domain, and one or more intracellular signaling domains,
wherein the scFv comprises a heavy chain variable (VH) region and a light chain variable (VL) region pair, and
wherein the VH and VL pair is selected from the group consisting of:
a. a VH region comprising a heavy chain complementarity determining region 1 (CDR-H1) having the amino acid sequence of SEQ ID NO: 1, a heavy chain complementarity determining region 2 (CDR-H2) having the amino acid sequence of SEQ ID NO: 2, a heavy chain complementarity determining region 3 (CDR-H3) having the amino acid sequence of SEQ ID NO: 3, and
a VL region comprising a light chain complementarity determining region 1 (CDR-L 1) having the amino acid sequence of SEQ ID NO: 58, a light chain complementarity determining region 2 (CDR-L2) having the amino acid sequence of SEQ ID NO: 59, and a light chain complementarity determining region 3 (CDR-L3) having the amino acid sequence of SEQ ID NO: 60;
b. a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 5, a heavy chain complementarity determining region 3 (CDR-H3) having the amino acid sequence of SEQ ID NO: 6, and
a VL region comprising a light chain complementarity determining region 1 (CDR-L 1) having the amino acid sequence of SEQ ID NO: 61, a light chain complementarity determining region 2 (CDR-L2) having the amino acid sequence of SEQ ID NO: 62, and a light chain complementarity determining region 3 (CDR-L3) having the amino acid sequence of SEQ ID NO: 63;
c. a VH region comprising a heavy chain complementarity determining region 1 (CDR-H1) having the amino acid sequence of SEQ ID NO: 7, a heavy chain complementarity determining region 2 (CDR-H2) having the amino acid sequence of SEQ ID NO: 8, a heavy chain complementarity determining region 3 (CDR-H3) having the amino acid sequence of SEQ ID NO: 9, and
a VL region comprising a light chain complementarity determining region 1 (CDR-L 1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 66;
d. a VH region comprising a heavy chain complementarity determining region 1 (CDR-H1) having the amino acid sequence of SEQ ID NO: 10, a heavy chain complementarity determining region 2 (CDR-H2) having the amino acid sequence of SEQ ID NO: 11, a heavy chain complementarity determining region 3 (CDR-H3) having the amino acid sequence of SEQ ID NO: 12, and
a VL region comprising a light chain complementarity determining region 1 (CDR-L 1) having the amino acid sequence of SEQ ID NO: 67, a light chain complementarity determining region 2 (CDR-L2) having the amino acid sequence of SEQ ID NO: 68, and a light chain complementarity determining region 3 (CDR-L3) having the amino acid sequence of SEQ ID NO: 69;
e. a VH region comprising a heavy chain complementarity determining region 1 (CDR-H1) having the amino acid sequence of SEQ ID NO: 13, a heavy chain complementarity determining region 2 (CDR-H2) having the amino acid sequence of SEQ ID NO: 14, a heavy chain complementarity determining region 3 (CDR-H3) having the amino acid sequence of SEQ ID NO: 15, and
a VL region comprising a light chain complementarity determining region 1 (CDR-L1) having the amino acid sequence of SEQ ID NO: 70, a light chain complementarity determining region 2 (CDR-L2) having the amino acid sequence of SEQ ID NO: 71, and a light chain complementarity determining region 3 (CDR-L3) having the amino acid sequence of SEQ ID NO: 72;
f. a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 17, a heavy chain complementarity determining region 3 (CDR-H3) having the amino acid sequence of SEQ ID NO: 18, and
a VL region comprising a light chain complementarity determining region 1 (CDR-L1) having the amino acid sequence of SEQ ID NO: 73, a light chain complementarity determining region 2 (CDR-L2) having the amino acid sequence of SEQ ID NO: 74, and a light chain complementarity determining region 3 (CDR-L3) having the amino acid sequence of SEQ ID NO: 75;
g. a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 19, a heavy chain complementarity determining region 3 (CDR-H3) having the amino acid sequence of SEQ ID NO: 20, and
a VL region comprising a light chain complementarity determining region 1 (CDR-L1) having the amino acid sequence of SEQ ID NO: 76, a light chain complementarity determining region 2 (CDR-L2) having the amino acid sequence of SEQ ID NO: 77, and a light chain complementarity determining region 3 (CDR-L3) having the amino acid sequence of SEQ ID NO: 78;
h. a VH region comprising a heavy chain complementarity determining region 1 (CDR-H1) having the amino acid sequence of SEQ ID NO: 21, a heavy chain complementarity determining region 2 (CDR-H2) having the amino acid sequence of SEQ ID NO: 22, a heavy chain complementarity determining region 3 (CDR-H3) having the amino acid sequence of SEQ ID NO: 23, and
a VL region comprising a light chain complementarity determining region 1 (CDR-L1) having the amino acid sequence of SEQ ID NO: 79, a light chain complementarity determining region 2 (CDR-L2) having the amino acid sequence of SEQ ID NO: 80, and a light chain complementarity determining region 3 (CDR-L3) having the amino acid sequence of SEQ ID NO: 81;
i. a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 24, a heavy chain complementarity determining region 3 (CDR-H3) having the amino acid sequence of SEQ ID NO: 25, and
a VL region comprising a light chain complementarity determining region 1 (CDR-L 1) having the amino acid sequence of SEQ ID NO: 82, a light chain complementarity determining region 2 (CDR-L2) having the amino acid sequence of SEQ ID NO: 65, and a light chain complementarity determining region 3 (CDR-L3) having the amino acid sequence of SEQ ID NO: 83;
j. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
k. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 85, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
l. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 86, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
m. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 87, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
n. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 88, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
o. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 89, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
p. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 90, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
q. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 91, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
r. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 92, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
s. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 93, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
t. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 94, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
u. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 95, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
v. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 96, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
w. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 97, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
x. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 98, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
y. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 99, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
z. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 100, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
aa. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L 1) having the amino acid sequence of SEQ ID NO: 101, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
bb. a VH region comprising a heavy chain complementarity determining region 1 (CDR-H1) having the amino acid sequence of SEQ ID NO: 29, a heavy chain complementarity determining region 2 (CDR-H2) having the amino acid sequence of SEQ ID NO: 30, a heavy chain complementarity determining region 3 (CDR-H3) having the amino acid sequence of SEQ ID NO: 31, and
a VL region comprising a light chain complementarity determining region 1 (CDR-L 1) having the amino acid sequence of SEQ ID NO: 102, a light chain complementarity determining region 2 (CDR-L2) having the amino acid sequence of SEQ ID NO: 103, and a light chain complementarity determining region 3 (CDR-L3) having the amino acid sequence of SEQ ID NO: 104;
cc. a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and
a VL region comprising a light chain complementarity determining region 1 (CDR-L 1) having the amino acid sequence of SEQ ID NO: 105, a light chain complementarity determining region 2 (CDR-L2) having the amino acid sequence of SEQ ID NO: 71, and a light chain complementarity determining region 3 (CDR-L3) having the amino acid sequence of SEQ ID NO: 106;
dd. a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and
a VL region comprising a light chain complementarity determining region 1 (CDR-L 1) having the amino acid sequence of SEQ ID NO: 107, a light chain complementarity determining region 2 (CDR-L2) having the amino acid sequence of SEQ ID NO: 108, and a light chain complementarity determining region 3 (CDR-L3) having the amino acid sequence of SEQ ID NO: 109;
ee. a VH region comprising a heavy chain complementarity determining region 1 (CDR-H1) having the amino acid sequence of SEQ ID NO: 145, a heavy chain complementarity determining region 2 (CDR-H2) having the amino acid sequence of SEQ ID NO: 146, a heavy chain complementarity determining region 3 (CDR-H3) having the amino acid sequence of SEQ ID NO: 147, and
a VL region comprising a light chain complementarity determining region 1 (CDR-L 1) having the amino acid sequence of SEQ ID NO: 155, a light chain complementarity determining region 2 (CDR-L2) having the amino acid sequence of SEQ ID NO: 156, and a light chain complementarity determining region 3 (CDR-L3) having the amino acid sequence of SEQ ID NO: 157;
ff. a VH region comprising a heavy chain complementarity determining region 1 (CDR-H1) having the amino acid sequence of SEQ ID NO: 148, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 147, and
a VL region comprising a light chain complementarity determining region 1 (CDR-L 1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157; and
gg. a VH region comprising a heavy chain complementarity determining region 1 (CDR-H1) having the amino acid sequence of SEQ ID NO: 150, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 151, and
a VL region comprising a light chain complementarity determining region 1 (CDR-L 1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157.

2. The CAR of embodiment 1, wherein the VH region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs: 36, 38, 39, 41, 43, 44, 45, 46, 47, 48, 49, 50, 152, 153, and 154.

3. The CAR of embodiment 1 or embodiment 2, wherein the VL region comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs: 111, 113, 114, 116, 118-143, and 159-161.

4. The CAR of any one of embodiments 1-3, wherein the VH region comprises the amino acid sequence of SEQ ID NO: 36 and the VL region comprises the amino acid sequence of SEQ ID NO: 111.

5. The CAR of any one of embodiments 1-3, wherein the VH region comprises the amino acid sequence of SEQ ID NO: 38 and the VL region comprises the amino acid sequence of SEQ ID NO: 113.

6. The CAR of any one of embodiments 1-3, wherein the VH region comprises the amino acid sequence of SEQ ID NO: 39 and the VL region comprises the amino acid sequence of SEQ ID NO: 114.

7. The CAR of any one of embodiments 1-3, wherein the VH region comprises the amino acid sequence of SEQ ID NO: 41 and the VL region comprises the amino acid sequence of SEQ ID NO: 116.

8. The CAR of any one of embodiments 1-3, wherein the VH region comprises the amino acid sequence of SEQ ID NO: 43 and the VL region comprises the amino acid sequence of SEQ ID NO: 118.

9. The CAR of any one of embodiments 1-3, wherein the VH region comprises the amino acid sequence of SEQ ID NO: 44 and the VL region comprises the amino acid sequence of SEQ ID NO: 119.

10. The CAR of any one of embodiments 1-3, wherein the VH region comprises the amino acid sequence of SEQ ID NO: 45 and the VL region comprises the amino acid sequence of SEQ ID NO: 120.

11. The CAR of any one of embodiments 1-3, wherein the VH region comprises the amino acid sequence of SEQ ID NO: 46 and the VL region comprises the amino acid sequence of SEQ ID NO: 121.

12. The CAR of any one of embodiments 1-3, wherein the VH region comprises the amino acid sequence of SEQ ID NO: 47 and the VL region comprises the amino acid sequence of SEQ ID NO: 122.

13. The CAR of any one of embodiments 1-3, wherein the VH region comprises the amino acid sequence of SEQ ID NO: 48 and the VL region comprises the amino acid sequence of SEQ ID NO: 123.

14. The CAR of any one of embodiments 1-3, wherein the VH region comprises the amino acid sequence of SEQ ID NO: 48 and the VL region comprises the amino acid sequence of SEQ ID NO: 124.

15. The CAR of any one of embodiments 1-3, wherein the VH region comprises the amino acid sequence of SEQ ID NO: 48 and the VL region comprises the amino acid sequence of SEQ ID NO: 125.

16. The CAR of any one of embodiments 1-3, wherein the VH region comprises the amino acid sequence of SEQ ID NO: 48 and the VL region comprises the amino acid sequence of SEQ ID NO: 126.

17. The CAR of any one of embodiments 1-3, wherein the VH region comprises the amino acid sequence of SEQ ID NO: 48 and the VL region comprises the amino acid sequence of SEQ ID NO: 127.

18. The CAR of any one of embodiments 1-3, wherein the VH region comprises the amino acid sequence of SEQ ID NO: 48 and the VL region comprises the amino acid sequence of SEQ ID NO: 128.

19. The CAR of any one of embodiments 1-3, wherein the VH region comprises the amino acid sequence of SEQ ID NO: 48 and the VL region comprises the amino acid sequence of SEQ ID NO: 129.

20. The CAR of any one of embodiments 1-3, wherein the VH region comprises the amino acid sequence of SEQ ID NO: 48 and the VL region comprises the amino acid sequence of SEQ ID NO: 130.

21. The CAR of any one of embodiments 1-3, wherein the VH region comprises the amino acid sequence of SEQ ID NO: 48 and the VL region comprises the amino acid sequence of SEQ ID NO: 131.

22. The CAR of any one of embodiments 1-3, wherein the VH region comprises the amino acid sequence of SEQ ID NO: 48 and the VL region comprises the amino acid sequence of SEQ ID NO: 132.

23. The CAR of any one of embodiments 1-3, wherein the VH region comprises the amino acid sequence of SEQ ID NO: 48 and the VL region comprises the amino acid sequence of SEQ ID NO: 133.

24. The CAR of any one of embodiments 1-3, wherein the VH region comprises the amino acid sequence of SEQ ID NO: 48 and the VL region comprises the amino acid sequence of SEQ ID NO: 134.

25. The CAR of any one of embodiments 1-3, wherein the VH region comprises the amino acid sequence of SEQ ID NO: 48 and the VL region comprises the amino acid sequence of SEQ ID NO: 135.

26. The CAR of any one of embodiments 1-3, wherein the VH region comprises the amino acid sequence of SEQ ID NO: 48 and the VL region comprises the amino acid sequence of SEQ ID NO: 136.

27. The CAR of any one of embodiments 1-3, wherein the VH region comprises the amino acid sequence of SEQ ID NO: 48 and the VL region comprises the amino acid sequence of SEQ ID NO: 137.

28. The CAR of any one of embodiments 1-3, wherein the VH region comprises the amino acid sequence of SEQ ID NO: 48 and the VL region comprises the amino acid sequence of SEQ ID NO: 138.

29. The CAR of any one of embodiments 1-3, wherein the VH region comprises the amino acid sequence of SEQ ID NO: 48 and the VL region comprises the amino acid sequence of SEQ ID NO: 139.

30. The CAR of any one of embodiments 1-3, wherein the VH region comprises the amino acid sequence of SEQ ID NO: 48 and the VL region comprises the amino acid sequence of SEQ ID NO: 140.

31. The CAR of any one of embodiments 1-3, wherein the VH region comprises the amino acid sequence of SEQ ID NO: 49 and the VL region comprises the amino acid sequence of SEQ ID NO: 141.

32. The CAR of any one of embodiments 1-3, wherein the VH region comprises the amino acid sequence of SEQ ID NO: 50 and the VL region comprises the amino acid sequence of SEQ ID NO: 142.

33. The CAR of any one of embodiments 1-3, wherein the VH region comprises the amino acid sequence of SEQ ID NO: 50 and the VL region comprises the amino acid sequence of SEQ ID NO: 143.

34. The CAR of any one of embodiments 1-3, wherein the VH region comprises the amino acid sequence of SEQ ID NO: 152 and the VL region comprises the amino acid sequence of SEQ ID NO: 159.

35. The CAR of any one of embodiments 1-3, wherein the VH region comprises the amino acid sequence of SEQ ID NO: 153 and the VL region comprises the amino acid sequence of SEQ ID NO: 160.

36. The CAR of any one of embodiments 1-3, wherein the VH region comprises the amino acid sequence of SEQ ID NO: 154 and the VL region comprises the amino acid sequence of SEQ ID NO: 161.

37. A chimeric antigen receptor (CAR) that binds to Glypican-3 (GPC3), wherein the CAR comprises a single chain Fv (scFv) that binds to GPC3, a transmembrane domain, and one or more intracellular signaling domains,
wherein the scFv comprises a heavy chain variable (VH) region and a light chain variable (VL) region,
wherein the VH comprises a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of an amino acid sequence selected from the group consisting of SEQ ID NO: 35-57 and 152-154; and
wherein the VL comprises a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of an amino acid sequence selected from the group consisting of SEQ ID NO: 110-144 and 159-161.

38. A chimeric antigen receptor (CAR) that binds to Glypican-3 (GPC3), wherein the CAR comprises a single chain Fv (scFv) that binds to GPC3, a transmembrane domain, and one or more intracellular signaling domains,
wherein the scFv comprises a heavy chain variable (VH) region and a light chain variable (VL) region,
wherein the VH comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 35-57 and 152-154; and
wherein the VL comprises an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 110-144 and 159-161.

39. A chimeric antigen receptor (CAR) that binds to Glypican-3 (GPC3), wherein the CAR comprises a single chain Fv (scFv) that binds to GPC3, a transmembrane domain, and one or more intracellular signaling domains,
wherein the scFv comprises a heavy chain variable (VH) region and a light chain variable (VL) region pair, and
wherein the VH and VL pair is selected from the group consisting of:
a. a VH region having the amino acid sequence of SEQ ID NO: 35 and VL region having the amino acid sequence of SEQ ID NO: 110;
b. a VH region having the amino acid sequence of SEQ ID NO: 36 and VL region having the amino acid sequence of SEQ ID NO: 111;
c. a VH region having the amino acid sequence of SEQ ID NO: 37 and VL region having the amino acid sequence of SEQ ID NO: 112;
d. a VH region having the amino acid sequence of SEQ ID NO: 38 and VL region having the amino acid sequence of SEQ ID NO: 113;
e. a VH region having the amino acid sequence of SEQ ID NO: 39 and VL region having the amino acid sequence of SEQ ID NO: 114;
f. a VH region having the amino acid sequence of SEQ ID NO: 40 and VL region having the amino acid sequence of SEQ ID NO: 115;
g. a VH region having the amino acid sequence of SEQ ID NO: 41 and VL region having the amino acid sequence of SEQ ID NO: 116;
h. a VH region having the amino acid sequence of SEQ ID NO: 42 and VL region having the amino acid sequence of SEQ ID NO: 117;
i. a VH region having the amino acid sequence of SEQ ID NO: 43 and VL region having the amino acid sequence of SEQ ID NO: 118;
j. a VH region having the amino acid sequence of SEQ ID NO: 44 and VL region having the amino acid sequence of SEQ ID NO: 119;
k. a VH region having the amino acid sequence of SEQ ID NO: 45 and VL region having the amino acid sequence of SEQ ID NO: 120;
l. a VH region having the amino acid sequence of SEQ ID NO: 46 and VL region having the amino acid sequence of SEQ ID NO: 121;
m. a VH region having the amino acid sequence of SEQ ID NO: 47 and VL region having the amino acid sequence of SEQ ID NO: 122;
n. a VH region having the amino acid sequence of SEQ ID NO: 48 and VL region having the amino acid sequence of SEQ ID NO: 123;
o. a VH region having the amino acid sequence of SEQ ID NO: 48 and VL region having the amino acid sequence of SEQ ID NO: 124;
p. a VH region having the amino acid sequence of SEQ ID NO: 48 and VL region having the amino acid sequence of SEQ ID NO: 125;
q. a VH region having the amino acid sequence of SEQ ID NO: 48 and VL region having the amino acid sequence of SEQ ID NO: 126;
r. a VH region having the amino acid sequence of SEQ ID NO: 48 and VL region having the amino acid sequence of SEQ ID NO: 127;
s. a VH region having the amino acid sequence of SEQ ID NO: 48 and VL region having the amino acid sequence of SEQ ID NO: 128;
t. a VH region having the amino acid sequence of SEQ ID NO: 48 and VL region having the amino acid sequence of SEQ ID NO: 129;
u. a VH region having the amino acid sequence of SEQ ID NO: 48 and VL region having the amino acid sequence of SEQ ID NO: 130;
v. a VH region having the amino acid sequence of SEQ ID NO: 48 and VL region having the amino acid sequence of SEQ ID NO: 131;
w. a VH region having the amino acid sequence of SEQ ID NO: 48 and VL region having the amino acid sequence of SEQ ID NO: 132;
x. a VH region having the amino acid sequence of SEQ ID NO: 48 and VL region having the amino acid sequence of SEQ ID NO: 133;
y. a VH region having the amino acid sequence of SEQ ID NO: 48 and VL region having the amino acid sequence of SEQ ID NO: 134;
z. a VH region having the amino acid sequence of SEQ ID NO: 48 and VL region having the amino acid sequence of SEQ ID NO: 135;
aa. a VH region having the amino acid sequence of SEQ ID NO: 48 and VL region having the amino acid sequence of SEQ ID NO: 136;
bb. a VH region having the amino acid sequence of SEQ ID NO: 48 and VL region having the amino acid sequence of SEQ ID NO: 137;
cc. a VH region having the amino acid sequence of SEQ ID NO: 48 and VL region having the amino acid sequence of SEQ ID NO: 138;
dd. a VH region having the amino acid sequence of SEQ ID NO: 48 and VL region having the amino acid sequence of SEQ ID NO: 139;
ee. a VH region having the amino acid sequence of SEQ ID NO: 48 and VL region having the amino acid sequence of SEQ ID NO: 140;
ff. a VH region having the amino acid sequence of SEQ ID NO: 49 and VL region having the amino acid sequence of SEQ ID NO: 141;
gg. a VH region having the amino acid sequence of SEQ ID NO: 50 and VL region having the amino acid sequence of SEQ ID NO: 142;
hh. a VH region having the amino acid sequence of SEQ ID NO: 50 and VL region having the amino acid sequence of SEQ ID NO: 143;
ii. a VH region having the amino acid sequence of SEQ ID NO: 51 and VL region having the amino acid sequence of SEQ ID NO: 144;
jj. a VH region having the amino acid sequence of SEQ ID NO: 52 and VL region having the amino acid sequence of SEQ ID NO: 144;
kk. a VH region having the amino acid sequence of SEQ ID NO: 53 and VL region having the amino acid sequence of SEQ ID NO: 144;
ll. a VH region having the amino acid sequence of SEQ ID NO: 54 and VL region having the amino acid sequence of SEQ ID NO: 144;
mm. a VH region having the amino acid sequence of SEQ ID NO: 55 and VL region having the amino acid sequence of SEQ ID NO: 144;
nn. a VH region having the amino acid sequence of SEQ ID NO: 56 and VL region having the amino acid sequence of SEQ ID NO: 144;
oo. a VH region having the amino acid sequence of SEQ ID NO: 57 and VL region having the amino acid sequence of SEQ ID NO: 144;
pp. a VH region having the amino acid sequence of SEQ ID NO: 152 and VL region having the amino acid sequence of SEQ ID NO: 159;
qq. a VH region having the amino acid sequence of SEQ ID NO: 153 and VL region having the amino acid sequence of SEQ ID NO: 160; and
rr. a VH region having the amino acid sequence of SEQ ID NO: 154 and VL region having the amino acid sequence of SEQ ID NO: 161.

40. The CAR of any one of embodiments 1-39, wherein the VH and VL of the scFv are separated by a peptide linker.

41. The CAR of any one of embodiments 1-40, wherein the scFV comprises the structure VH-L-VL or VL-L-VH, wherein VH is the heavy chain variable region, L is the peptide linker, and VL is the light chain variable region.

42. The CAR of embodiment 40 of embodiment 41, wherein the peptide linker comprises an amino acid sequence selected from the group consisting of SEQ ID No: 162-180.

43. The CAR of any one of embodiments 1-42, wherein the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2 DS1 transmembrane domain, a KIR3 DS1 transmembrane domain, an NKp44 transmembrane domain, an NKp46 transmembrane domain, an FceRlg transmembrane domain, and an NKG2D transmembrane domain.

44. The CAR of any one of embodiments 1-43, wherein the one or more intracellular signaling domains are each selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2 DS1 intracellular signaling domain, a KIR3 DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

45. The CAR of any one of embodiments 1-44, wherein the CAR comprises one or more of a hinge domain, a spacer region, or one or more peptide linkers.

46. The CAR of any one of embodiments 1-45, wherein the CAR comprises a spacer region between the scFV and the transmembrane domain.

47. The CAR of embodiment 41, wherein the spacer region has an amino acid sequence selected from the group consisting of SEQ ID NOs: 181-90.

48. A composition comprising the CAR of any one of embodiments 1-47 and a pharmaceutically acceptable carrier, pharmaceutically acceptable excipient, or a combination thereof.

49. An engineered nucleic acid encoding the CAR of any one of embodiments 1-47.

50. An expression vector comprising the engineered nucleic acid of embodiment 49.

51. A composition comprising the engineered nucleic acid of embodiment 49 or the expression vector of embodiment 50, and a pharmaceutically acceptable carrier, pharmaceutically acceptable excipient, or a combination thereof.

52. A method of making an engineered cell, comprising transducing an isolated cell with the engineered nucleic acid of embodiment 49 or the expression vector of embodiment 50.

53. An engineered cell produced by the method of embodiment 52.

54. An isolated cell comprising the engineered nucleic acid of embodiment 49, the expression vector of embodiment 50, or the composition of embodiment 51.

55. A population of engineered cells expressing the engineered nucleic acid of embodiment 49 or the expression vector of embodiment 50.

56. An isolated cell comprising the CAR of any one of embodiments 1-47.

57. A population of engineered cells expressing the CAR of any one of embodiments 1-47.

58. The cell or population of cells of any one of embodiments 53-57, wherein the CAR is recombinantly expressed.

59. The cell or population of cells of any one of embodiments 53-58, wherein the CAR is expressed from a vector or a selected locus from the genome of the cell.

60. The cell or population of cells of any one of embodiments 53-59, wherein the cell or population of cells further expresses one or more immunomodulating effectors.

61. The cell or population of cells of embodiment 60, wherein the one or more immunomodulating effectors are one or more cytokines or chemokines.

62. The cell or population of cells of embodiment 61, wherein the one or more cytokines or chemokines are selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, TNF-alpha, CCL21a, CXCL10, CXCL11, CXCL13, a CXCL10-CXCL11 fusion protein, CCL19, CXCL9, and XCL1.

63. The cell or population of cells of any one of embodiments 60-62, wherein expression of the one or more immunomodulating effectors is controlled by an activation-conditional control polypeptide (ACP).

64. The cell or population of cells of embodiment 63, wherein the one or more immunomodulating effectors are expressed from one or more expression cassettes, wherein the one or more expression cassettes each comprises an ACP-responsive promoter and an exogenous polynucleotide sequence encoding one or more immunomodulating effectors, wherein the ACP-responsive promoter is operably linked to the exogenous polynucleotide.

65. The cell or population of cells of embodiment 64, wherein the ACP is capable of inducing expression of the one or more expression cassettes by binding to the ACP-responsive promoter.

66. The cell or population of cells of embodiment 64 or embodiment 65, wherein the ACP-responsive promoter comprises an ACP-binding domain and a promoter sequence.

67. The cell or population of cells of embodiment 66, wherein the promoter sequence is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB_TATA, minTATA, minTK, inducer molecule responsive promoters, and tandem repeats thereof.

68. The cell or population of cells of any one of embodiments 64-67, wherein the ACP-responsive promoter is a synthetic promoter.

69. The cell or population of cells of any one of embodiments 64-68, wherein the ACP-responsive promoter comprises a minimal promoter.

70. The cell or population of cells of any one of embodiments 64-69, wherein the ACP-binding domain comprises one or more zinc finger binding sites.

71. The cell or population of cells of any one of embodiments 64-70, wherein the ACP is a transcriptional modulator.

72. The cell or population of cells of any one of embodiments 64-71, wherein the ACP is a transcriptional repressor.

73. The cell or population of cells of any one of embodiments 64-71, wherein the ACP is a transcriptional activator.

74. The cell or population of cells of any one of embodiments 64-73, wherein the ACP further comprises a repressible protease and one or more cognate cleavage sites of the repressible protease.

75. The cell or population of cells of any one of embodiments 64-73, wherein the ACP further comprises a hormone-binding domain of estrogen receptor (ERT2 domain).

76. The cell or population of cells of any one of embodiments 64-75, wherein the ACP is a transcription factor.

77. The cell or population of cells of embodiment 76, wherein the ACP is a zinc-finger-containing transcription factor.

78. The cell or population of cells of embodiment 77, wherein the zinc finger-containing transcription factor comprises a DNA-binding zinc finger protein domain (ZF protein domain) and an effector domain.

79. The cell or population of cells of embodiment 78, wherein the ZF protein domain is modular in design and is composed of zinc finger arrays (ZFA).

80. The cell or population of cells of embodiment 79, wherein the ZF protein domain comprises one to ten ZFA.

81. The cell or population of cells of any one of embodiments 7880, wherein the effector domain is selected from the group consisting of: a Herpes Simplex Virus Protein 16 (VP16) activation domain; an activation domain comprising four tandem copies of VP16, a VP64 activation domain; a p65 activation domain of NF [symbol] EƒB; an Epstein-Barr virus R transactivator (Rta) activation domain; a tripartite activator comprising the VP64, the p65, and the Rta activation domains (VPR activation domain); a tripartite activator comprising the VP64, the p65, and the HSP90 activation domains (VPH activation domain); a histone acetyltransferase (HAT) core domain of the human E1A-associated protein p300 (p300 HAT core activation domain); a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

82. The cell or population of cells of any one of embodiments 74-81, wherein the one or more cognate cleavage sites of the repressible protease are localized between the ZF protein domain and the effector domain.

83. The cell or population of cells of any one of embodiments 74-82, wherein the repressible protease is a hepatitis C virus (HCV) nonstructural protein 3 (NS3).

84. The cell or population of cells of embodiment 83, wherein the cognate cleavage site comprises an NS3 protease cleavage site.

85. The cell or population of cells of embodiment 84, wherein the NS3 protease cleavage site comprises a NS3/NS4A, a NS4A/NS4B, a NS4B/NS5A, or a NS5A/NS5B junction cleavage site.

86. The cell or population of cells of any one of embodiments 83-85, wherein the NS3 protease can be repressed by a protease inhibitor.

87. The cell or population of cells of embodiment 86, wherein the protease inhibitor is selected from the group consisting of: simeprevir, danoprevir, asunaprevir, ciluprevir, boceprevir, sovaprevir, paritaprevir, telaprevir, grazoprevir, glecaprevir, and voxiloprevir.

88. The cell or population of cells of embodiment 86, wherein the protease inhibitor comprises grazoprevir.

89. The cell or population of cells of any one of embodiments 75-88, wherein the ACP is capable of undergoing nuclear localization upon binding of the ERT2 domain to tamoxifen or a metabolite thereof.

90. The cell or population of cells of embodiment 89, wherein the tamoxifen metabolite is selected from the group consisting of: 4-hydroxytamoxifen, N-desmethyltamoxifen, tamoxifen-N-oxide, and endoxifen.

91. The cell or population of cells of any one of embodiments 63-90, wherein the ACP further comprises a degron, and wherein the degron is operably linked to the ACP.

92. The cell or population of cells of embodiment 91, wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of omithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

93. The cell or population of cells of embodiment 91, wherein the degron comprises a cereblon (CRBN) polypeptide substrate domain capable of binding CRBN in response to an immunomodulatory drug (IMiD) thereby promoting ubiquitin pathway-mediated degradation of the ACP.

94. The cell or population of cells of embodiment 93, wherein the CRBN polypeptide substrate domain is selected from the group consisting of: IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

95. The cell or population of cells of embodiment 93, wherein the CRBN polypeptide substrate domain is a chimeric fusion product of native CRBN polypeptide sequences.

96. The cell or population of cells of embodiment 93, wherein the CRBN polypeptide substrate domain is a IKZF3/ZFP91/IKZF3 chimeric fusion product having the amino acid sequence of FNVLM VHKRS HTGER PLQCE ICGFT CRQKG NLLRH IKLHT GEKPF KCHLC NYACQ RRDAL.

97. The cell or population of cells of any one of embodiments 93-96, wherein the IMiD is an FDA-approved drug.

98. The cell or population of cells of any one of embodiments 93-97, wherein the IMiD is selected from the group consisting of: thalidomide, lenalidomide, and pomalidomide.

99. The cell or population of cells of any one of embodiments 91-99, wherein the degron is localized 5′ of the repressible protease, 3′ of the repressible protease, 5′ of the ZF protein domain, 3′ of the ZF protein domain, 5′ of the effector domain, or 3′ of the effector domain.

100. The cell or population of cells of any one of embodiments 53-99, wherein the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

101. The cell or population of cells of any one of embodiments 53-100, wherein the cell or population of cells is a Natural Killer (NK) cell.

102. The cell or population of cells of any one of embodiments 53-101, wherein the cell or population of cells is autologous.

103. The cell or population of cells of any one of embodiments 53-101, wherein the cell or population of cells is allogeneic.

104. A pharmaceutical composition comprising an effective amount of the cell or population of engineered cells of any one of embodiments 53-103 and a pharmaceutically acceptable carrier, pharmaceutically acceptable excipient, or a combination thereof.

105. A pharmaceutical composition comprising an effective amount of genetically modified cells expressing the CAR of any one of embodiments 1-47 and a pharmaceutically acceptable carrier, pharmaceutically acceptable excipient, or a combination thereof.

106. The pharmaceutical composition of embodiment 104 or embodiment 105, which is for treating and/or preventing a tumor.

107. A method of treating a subject in need thereof, the method comprising administering a therapeutically effective dose of the composition of embodiment 43 or embodiment 46, or any of the cells of any one of embodiments 48-59, or the composition of embodiment 104 or embodiment 105.

108. A method of stimulating a cell-mediated immune response to a tumor cell in a subject, the method comprising administering to a subject having a tumor a therapeutically effective dose of the composition of embodiment 48 or embodiment 51, or any of the cells of any one of embodiments 53-103, or the composition of embodiment 104 or embodiment 105.

109. A method of treating a subject having a tumor, the method comprising administering a therapeutically effective dose of the composition of embodiment 48 or embodiment 51, or any of the cells of any one of embodiments 53-103, or the composition of embodiment 60 or embodiment 61.

110. A kit for treating and/or preventing a tumor, comprising the CAR of any one of embodiments 1-47.

111. The kit of embodiment 110, wherein the kit further comprises written instructions for using the chimeric protein for producing one or more antigen-specific cells for treating and/or preventing a tumor in a subject.

112. A kit for treating and/or preventing a tumor, comprising the cell or population of cells of any one of embodiments 53-103.

113. The kit of embodiment 112, wherein the kit further comprises written instructions for using the cell for treating and/or preventing a tumor in a subject.

114. A kit for treating and/or preventing a tumor, comprising the isolated nucleic acid of embodiment 49.

115. The kit of embodiment 114, wherein the kit further comprises written instructions for using the nucleic acid for producing one or more antigen-specific cells for treating and/or preventing a tumor in a subject.

116. A kit for treating and/or preventing a tumor, comprising the vector of embodiment 50.

117. The kit of embodiment 116, wherein the kit further comprises written instructions for using the vector for producing one or more antigen-specific cells for treating and/or preventing a tumor in a subject.

118. A kit for treating and/or preventing a tumor, comprising the composition of any one of embodiments 48, 51, 104, or 105.

119. The kit of embodiment 118, wherein the kit further comprises written instructions for using the composition for treating and/or preventing a tumor in a subject.

1. A chimeric antigen receptor (CAR) that binds to Glypican-3 (GPC3), wherein the CAR comprises a single chain Fv (scFv) that binds to GPC3, a transmembrane domain, and one or more intracellular signaling domains,
wherein the scFv comprises a heavy chain variable (VH) region and a light chain variable (VL) region pair, and
wherein
(a) the VH and VL pair is selected from the group consisting of:
i. a VH region comprising a heavy chain complementarity determining region 1 (CDR-H1) having the amino acid sequence of SEQ ID NO: 1, a heavy chain complementarity determining region 2 (CDR-H2) having the amino acid sequence of SEQ ID NO: 2, a heavy chain complementarity determining region 3 (CDR-H3) having the amino acid sequence of SEQ ID NO: 3, and
a VL region comprising a light chain complementarity determining region 1 (CDR-L1) having the amino acid sequence of SEQ ID NO: 58, a light chain complementarity determining region 2 (CDR-L2) having the amino acid sequence of SEQ ID NO: 59, and a light chain complementarity determining region 3 (CDR-L3) having the amino acid sequence of SEQ ID NO: 60;
ii. a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 5, a heavy chain complementarity determining region 3 (CDR-H3) having the amino acid sequence of SEQ ID NO: 6, and
a VL region comprising a light chain complementarity determining region 1 (CDR-L1) having the amino acid sequence of SEQ ID NO: 61, a light chain complementarity determining region 2 (CDR-L2) having the amino acid sequence of SEQ ID NO: 62, and a light chain complementarity determining region 3 (CDR-L3) having the amino acid sequence of SEQ ID NO: 63;
iii. a VH region comprising a heavy chain complementarity determining region 1 (CDR-H1) having the amino acid sequence of SEQ ID NO: 7, a heavy chain complementarity determining region 2 (CDR-H2) having the amino acid sequence of SEQ ID NO: 8, a heavy chain complementarity determining region 3 (CDR-H3) having the amino acid sequence of SEQ ID NO: 9, and
a VL region comprising a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 66;
iv. a VH region comprising a heavy chain complementarity determining region 1 (CDR-H1) having the amino acid sequence of SEQ ID NO: 10, a heavy chain complementarity determining region 2 (CDR-H2) having the amino acid sequence of SEQ ID NO: 11, a heavy chain complementarity determining region 3 (CDR-H3) having the amino acid sequence of SEQ ID NO: 12, and
a VL region comprising a light chain complementarity determining region 1 (CDR-L1) having the amino acid sequence of SEQ ID NO: 67, a light chain complementarity determining region 2 (CDR-L2) having the amino acid sequence of SEQ ID NO: 68, and a light chain complementarity determining region 3 (CDR-L3) having the amino acid sequence of SEQ ID NO: 69;
v. a VH region comprising a heavy chain complementarity determining region 1 (CDR-H1) having the amino acid sequence of SEQ ID NO: 13, a heavy chain complementarity determining region 2 (CDR-H2) having the amino acid sequence of SEQ ID NO: 14, a heavy chain complementarity determining region 3 (CDR-H3) having the amino acid sequence of SEQ ID NO: 15, and
a VL region comprising a light chain complementarity determining region 1 (CDR-L1) having the amino acid sequence of SEQ ID NO: 70, a light chain complementarity determining region 2 (CDR-L2) having the amino acid sequence of SEQ ID NO: 71, and a light chain complementarity determining region 3 (CDR-L3) having the amino acid sequence of SEQ ID NO: 72;
vi. a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 17, a heavy chain complementarity determining region 3 (CDR-H3) having the amino acid sequence of SEQ ID NO: 18, and
a VL region comprising a light chain complementarity determining region 1 (CDR-L1) having the amino acid sequence of SEQ ID NO: 73, a light chain complementarity determining region 2 (CDR-L2) having the amino acid sequence of SEQ ID NO: 74, and a light chain complementarity determining region 3 (CDR-L3) having the amino acid sequence of SEQ ID NO: 75;
vii. a VH region comprising a heavy chain complementarity determining region 1 (CDR-H1) having the amino acid sequence of SEQ ID NO: 16, a heavy chain complementarity determining region 2 (CDR-H2) having the amino acid sequence of SEQ ID NO: 19, a heavy chain complementarity determining region 3 (CDR-H3) having the amino acid sequence of SEQ ID NO: 20, and
a VL region comprising a light chain complementarity determining region 1 (CDR-L1) having the amino acid sequence of SEQ ID NO: 76, a light chain complementarity determining region 2 (CDR-L2) having the amino acid sequence of SEQ ID NO: 77, and a light chain complementarity determining region 3 (CDR-L3) having the amino acid sequence of SEQ ID NO: 78;
viii. a VH region comprising a heavy chain complementarity determining region 1 (CDR-H1) having the amino acid sequence of SEQ ID NO: 21, a heavy chain complementarity determining region 2 (CDR-H2) having the amino acid sequence of SEQ ID NO: 22, a heavy chain complementarity determining region 3 (CDR-H3) having the amino acid sequence of SEQ ID NO: 23, and
a VL region comprising a light chain complementarity determining region 1 (CDR-L1) having the amino acid sequence of SEQ ID NO: 79, a light chain complementarity determining region 2 (CDR-L2) having the amino acid sequence of SEQ ID NO: 80, and a light chain complementarity determining region 3 (CDR-L3) having the amino acid sequence of SEQ ID NO: 81;
ix. a VH region comprising a heavy chain complementarity determining region 1 (CDR-H1) having the amino acid sequence of SEQ ID NO: 4, a heavy chain complementarity determining region 2 (CDR-H2) having the amino acid sequence of SEQ ID NO: 24, a heavy chain complementarity determining region 3 (CDR-H3) having the amino acid sequence of SEQ ID NO: 25, and
a VL region comprising a light chain complementarity determining region 1 (CDR-L1) having the amino acid sequence of SEQ ID NO: 82, a light chain complementarity determining region 2 (CDR-L2) having the amino acid sequence of SEQ ID NO: 65, and a light chain complementarity determining region 3 (CDR-L3) having the amino acid sequence of SEQ ID NO: 83;
x. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 64, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xi. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 85, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 86, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xiii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 87, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xiv. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 88, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xv. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 89, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xvi. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 90, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xvii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 91, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xviii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 92, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xix. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 93, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xx. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 94, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxi. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 95, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 96, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxiii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 97, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxiv. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 98, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxv. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 99, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxvi. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 100, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxvii. a VH region comprising a heavy chain complementarity determining region 1 (CDR-H1) having the amino acid sequence of SEQ ID NO: 26, a heavy chain complementarity determining region 2 (CDR-H2) having the amino acid sequence of SEQ ID NO: 27, a heavy chain complementarity determining region 3 (CDR-H3) having the amino acid sequence of SEQ ID NO: 28, and
a VL region comprising a light chain complementarity determining region 1 (CDR-L1) having the amino acid sequence of SEQ ID NO: 101, a light chain complementarity determining region 2 (CDR-L2) having the amino acid sequence of SEQ ID NO: 65, and a light chain complementarity determining region 3 (CDR-L3) having the amino acid sequence of SEQ ID NO: 84;
xxviii. a VH region comprising a heavy chain complementarity determining region 1 (CDR-H1) having the amino acid sequence of SEQ ID NO: 29, a heavy chain complementarity determining region 2 (CDR-H2) having the amino acid sequence of SEQ ID NO: 30, a heavy chain complementarity determining region 3 (CDR-H3) having the amino acid sequence of SEQ ID NO: 31, and
a VL region comprising a light chain complementarity determining region 1 (CDR-L1) having the amino acid sequence of SEQ ID NO: 102, a light chain complementarity determining region 2 (CDR-L2) having the amino acid sequence of SEQ ID NO: 103, and a light chain complementarity determining region 3 (CDR-L3) having the amino acid sequence of SEQ ID NO: 104;
xxix. a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and
a VL region comprising a light chain complementarity determining region 1 (CDR-L1) having the amino acid sequence of SEQ ID NO: 105, a light chain complementarity determining region 2 (CDR-L2) having the amino acid sequence of SEQ ID NO: 71, and a light chain complementarity determining region 3 (CDR-L3) having the amino acid sequence of SEQ ID NO: 106;
xxx. a VH region comprising a heavy chain complementarity determining region 1 (CDR-H1) having the amino acid sequence of SEQ ID NO: 32, a heavy chain complementarity determining region 2 (CDR-H2) having the amino acid sequence of SEQ ID NO: 33, a heavy chain complementarity determining region 3 (CDR-H3) having the amino acid sequence of SEQ ID NO: 34, and
a VL region comprising a light chain complementarity determining region 1 (CDR-L1) having the amino acid sequence of SEQ ID NO: 107, a light chain complementarity determining region 2 (CDR-L2) having the amino acid sequence of SEQ ID NO: 108, and a light chain complementarity determining region 3 (CDR-L3) having the amino acid sequence of SEQ ID NO: 109;
xxxi. a VH region comprising a heavy chain complementarity determining region 1 (CDR-H1) having the amino acid sequence of SEQ ID NO: 145, a heavy chain complementarity determining region 2 (CDR-H2) having the amino acid sequence of SEQ ID NO: 146, a heavy chain complementarity determining region 3 (CDR-H3) having the amino acid sequence of SEQ ID NO: 147, and
a VL region comprising a light chain complementarity determining region 1 (CDR-L1) having the amino acid sequence of SEQ ID NO: 155, a light chain complementarity determining region 2 (CDR-L2) having the amino acid sequence of SEQ ID NO: 156, and a light chain complementarity determining region 3 (CDR-L3) having the amino acid sequence of SEQ ID NO: 157;
xxxii. a VH region comprising a heavy chain complementarity determining region 1 (CDR-H1) having the amino acid sequence of SEQ ID NO: 148, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 147, and
a VL region comprising a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157; and
xxxiii. a VH region comprising a heavy chain complementarity determining region 1 (CDR-H1) having the amino acid sequence of SEQ ID NO: 150, a heavy chain complementarity determining region 2 (CDR-H2) having the amino acid sequence of SEQ ID NO: 149, a heavy chain complementarity determining region 3 (CDR-H3) having the amino acid sequence of SEQ ID NO: 151, and
a VL region comprising a light chain complementarity determining region 1 (CDR-L1) having the amino acid sequence of SEQ ID NO: 158, a light chain complementarity determining region 2 (CDR-L2) having the amino acid sequence of SEQ ID NO: 156, and a light chain complementarity determining region 3 (CDR-L3) having the amino acid sequence of SEQ ID NO: 157, or
(b) the VH comprises:
a heavy chain complementarity determining region 1 (CDR-H1), a heavy chain complementarity determining region 2 (CDR-H2), and a heavy chain complementarity determining region 3 (CDR-H3), wherein the amino acid sequences of CDR-H1, CDR-H2, and CDR-H3 are contained within the VH region of an amino acid sequence selected from the group consisting of SEQ ID NO: 35-57 and 152-154; and
the VL comprises:
a light chain complementarity determining region 1 (CDR-L1), a light chain complementarity determining region 2 (CDR-L2), and a light chain complementarity determining region 3 (CDR-L3), and wherein the amino acid sequences of CDR-L1, CDR-L2, and CDR-L3 are contained within the VL region of an amino acid sequence selected from the group consisting of SEQ ID NO: 110-144 and 159-161, or
(c) the VH comprises:
an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 35-57 and 152-154; and
the VL comprises:
an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NO: 110-144 and 159-161, or
(d) the VH region comprises:
an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs:
36, 38, 39, 41, 43, 44, 45, 46, 47, 48, 49, 50, 152, 153, and 154, and
the VL region comprises:
an amino acid sequence with at least 90%, at least 91%, at least 92%, at least 93%, at least 94%, at least 95%, at least 96%, at least 97%, at least 98%, at least 99%, or 100% identity to an amino acid sequence selected from the group consisting of SEQ ID NOs: 111, 113, 114, 116, 118-143, and 159-161,
optionally wherein the CAR comprises one or more of a hinge domain, a spacer region, or one or more peptide linkers.

2. The CAR of claim wherein:
(a) the transmembrane domain is selected from the group consisting of: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2 DS1 transmembrane domain, a KIR3 DS1 transmembrane domain, an NKp44 transmembrane domain, an NKp46 transmembrane domain, an FceR1g transmembrane domain, and an NKG2D transmembrane domain; and/or
(b) the one or more intracellular signaling domains are each selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2 DS1 intracellular signaling domain, a KIR3 DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

3. The CAR of claim 1 or claim 2, wherein the VH and VL of the scFv are separated by a peptide linker,
optionally wherein the scFV comprises the structure VH-L-VL or VL-L-VH, wherein VH is the heavy chain variable region, L is the peptide linker, and VL is the light chain variable region.

4. An engineered nucleic acid encoding the CAR of any one of claims 1-3.

5. An expression vector comprising the engineered nucleic acid of claim 4.

6. An isolated cell comprising the CAR of any one of claims 1-3, the engineered nucleic acid of claim 4, or the expression vector of claim 5.

7. A population of engineered cells expressing the CAR of any one of claims 1-3, the engineered nucleic acid of claim 4, or the expression vector of claim 5.

8. The cell or population of cells of claim 6 or claim 7, wherein the CAR is recombinantly expressed, optionally wherein the CAR is expressed from a vector or a selected locus from the genome of the cell.

9. The cell or population of cells of any one of claims 6-8, wherein the cell or population of cells further expresses one or more immunomodulating effectors,
optionally wherein the one or more immunomodulating effectors are one or more cytokines or chemokines,
optionally wherein the one or more cytokines or chemokines are selected from the group consisting of: IL1-beta, IL2, IL4, IL6, IL7, IL10, IL12, an IL12p70 fusion protein, IL15, IL17A, 118, IL21, IL22, Type I interferons, Interferon-gamma, TNF-alpha, CCL21a, CXCL10, CXCL11, CXCL13, a CXCL10-CXCL11 fusion protein, CCL19, CXCL9, and XCL1.

10. The cell or population of cells of any one of claims 6-9, wherein expression of the one or more immunomodulating effectors is: (a) controlled by an activation-conditional control polypeptide (ACP), or (b) expressed from one or more expression cassettes, wherein the one or more expression cassettes each comprises an ACP-responsive promoter and an exogenous polynucleotide sequence encoding one or more immunomodulating effectors, wherein the ACP-responsive promoter is operably linked to the exogenous polynucleotide, optionally wherein the ACP is capable of inducing expression of the one or more expression cassettes by binding to the ACP-responsive promoter,
optionally wherein the ACP-responsive promoter comprises an ACP-binding domain and a promoter sequence,
optionally wherein the ACP-responsive promoter is a synthetic promoter or a minimal promoter,
optionally wherein the ACP is a transcriptional modulator, a transcriptional repressor, a transcriptional activator, a transcription factor, or a zinc-finger-containing transcription factor, optionally wherein the zinc finger-containing transcription factor comprises a DNA-binding zinc finger protein domain (ZF protein domain) and an effector domain, or optionally wherein the ACP is capable of undergoing nuclear localization upon binding of the ERT2 domain to tamoxifen or a metabolite thereof, or
optionally wherein the ACP further comprises (a) a repressible protease and one or more cognate cleavage sites of the repressible protease, (b) a hormone-binding domain of estrogen receptor (ERT2 domain), or (c) a degron, wherein the degron is operably linked to the ACP, optionally wherein the degron comprises a cereblon (CRBN) polypeptide substrate domain capable of binding CRBN in response to an immunomodulatory drug (IMiD) thereby promoting ubiquitin pathway-mediated degradation of the ACP.

11. The cell or population of cells of any one of claims 6-10, wherein the cell or population of cells is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell,
optionally wherein the cell or population of cells is autologous or allogeneic.

12. A pharmaceutical composition comprising an effective amount of the CAR of any one of claims 1-3, the engineered nucleic acid of claim 4, the expression vector of claim 5, or the cell or population of cells of any one of claims 7-11, and a pharmaceutically acceptable carrier, pharmaceutically acceptable excipient, or a combination thereof.

13. A method of stimulating a cell-mediated immune response to a tumor cell in a subject, the method comprising administering to a subject having a tumor a therapeutically effective dose of the CAR of any one of claims 1-3, the engineered nucleic acid of claim 4, the expression vector of claim 5, any of the cells of any one of claims 7-11, or the composition of claim 12.

14. A method of treating a subject having a tumor, the method comprising administering a therapeutically effective dose of the CAR of any one of claims 1-3, the engineered nucleic acid of claim 4, the expression vector of claim 5, any of the cells of any one of claims 7-11, or the composition of claim 12.

15. A kit for treating and/or preventing a tumor, comprising the CAR of any one of claims 1-3, the engineered nucleic acid of claim 4, the expression vector of claim 5, any of the cells of any one of claims 7-11, or the composition of claim 12.
